(19) United States
(12) Patent Application Publication
(10) Pub. No.: US 2025/0177405 A1
(43) Pub. Date: Jun. 5, 2025

(54) METHODS OF TREATING NEUROLOGICAL AND CARDIOVASCULAR CONDITIONS

(71) Applicant: ASTROCYTE PHARMACEUTICALS, INC., Cambridge, MA (US)

(72) Inventor: Theodore E. Liston, Stonington, CT (US)

(21) Appl. No.: 18/711,834

(22) PCT Filed: Nov. 22, 2022
(86) PCT No.: PCT/US2022/050743

§ 371 Date: May 20, 2024

Related U.S. Application Data
(60) Provisional application No. 63/264,424, filed on Nov. 22, 2021.

(51) Int. Cl. A61K 31/52; A61K 9/00; A61P 9/10; A61P 25/00

(52) U.S. Cl. CPC A61K 31/52; A61K 9/0019; A61P 9/10; A61P 25/00

(57) ABSTRACT

The present invention provides nucleoside analog compounds and methods of use thereof for treatment of certain injuries, diseases, disorders, and conditions, for example brain injuries such as stroke or traumatic brain injuries. The present invention further provides methods of screening adenosine receptor agonists for efficacy, determining optimal treatment regimens, and to evaluate brain and/or CNS receptor occupancy levels required for efficacy.

### CROSS-REFERENCE TO RELATED APPLICATIONS

This application claims the benefit under 35 U.S.C § 119(e) of U.S. Provisional Application No. 63/264,424, filed on Nov. 22, 2021, the entirety of which is incorporated herein by reference.

### FIELD OF THE INVENTION

The present invention relates to compounds and methods of use thereof for treating, ameliorating, or promoting recovery from certain conditions of the brain, central nervous system (CNS), or cardiovascular system such as a brain injury, a neurodegenerative condition, or cardiac ischemia. The present invention also provides methods of determining effective treatment dosing for such conditions.

### BACKGROUND OF THE INVENTION

Brain injuries and injury to the Central Nervous System (CNS) are a substantial cause of death and disability worldwide. Brain and CNS conditions that result in nerve cell death and damage range from ischemic episodes (e.g., stroke) and trauma, to degenerative disorders (e.g., Alzheimer's disease). Due to factors such as the inaccessibility of the brain and the limited capacity of neurons and other brain cells to regenerate, treatment options are limited. For example, current acute ischemic stroke (AIS) therapy is limited to recanalization by thrombolysis or thrombectomy. These therapies focus on restoring blood flow and oxygenation of hypoperfused tissue. Thrombolytics, however, can only be given to <5% of AIS patients within a limited time window post-occlusion, while thrombectomy requires access to the site of occlusion and is currently utilized in less than 20% of AIS patients.

Pharmacotherapy that is both cerebroprotective and administrable to a majority of AIS patients in conjunction with recanalization is a major unmet need. The vast majority of previous preclinical neuroprotection programs focused on efficacy evaluations in rodent models, with limited insights into drug distribution, target engagement and pharmacological response, perhaps inevitably leading to neutral or negative findings during clinical trials. For these reasons, the international Stroke Treatment Academic Industry Roundtable (STAIR) has issued detailed guidelines on preclinical testing of potential AIS therapies, including evaluation of efficacy in both lyssencephalic and gyrencephalic species. To date, there is a lack of options beyond the postsynaptic density protein-95 inhibitor nerinetide and the plasminogen modulator SMTP-7 that fulfill STAIR guidelines, for evaluation in both rodent and nonhuman primate (NHP) stroke models, prior to initiation of clinical trials. More generally, there is a need for more effective means of determining the effective dose—and whether an effective dose exists—for candidate compounds under investigation for stroke, TBI, and conditions of the brain, CNS, and cardiovascular system. For example, candidate compounds may need to cross the blood-brain barrier and engage with target receptors with sufficient efficiency in order to show effectiveness.

There is urgent and compelling unmet medical need for more effective treatments for brain injuries, CNS injuries, heart and cardiovascular diseases, and related conditions, as well as promoting neurorestoration in patients having a neurodegenerative condition such as Alzheimer's.

### SUMMARY OF THE INVENTION

In some aspects, the present invention provides methods of evaluating the effectiveness of new drug candidates, selecting clinical doses, improving efficacy, and of treating various injuries, diseases, disorders, and conditions. Such drug candidates include the compounds described herein. In one aspect, compounds for use in a provided method are A₁R and/or A₃R agonists.

In some embodiments, the compound acts as an agonist of an A₃ adenosine receptor (A₃R). In some embodiments, the compound is a partial A₃R agonist. In some embodiments, the compound is a biased A₃R agonist. In some embodiments, the compound acts by dual agonism at an A₃ adenosine receptor and an A₁ adenosine receptor (A₁R). In some embodiments, the compound acts as an agonist of an A₁ adenosine receptor (A₁R). In some embodiments, the compound is a partial A₁R agonist. In some embodiments, the compound is a biased A₁R agonist.

### BRIEF DESCRIPTION OF THE DRAWINGS

FIG. 1A-C show the effect of I-1 treatment initiation on the slope of DWI lesion growth following tMCAO in nonhuman primates. (A) Slopes of MCAO lesion growth prior to initiation of vehicle or I-1 treatment. (B) Slopes of MCAO lesion growth following vehicle or I-1 treatment. (C) Comparison of lesion growth slopes before and after initiation of vehicle or I-1 treatment 2 hours post-occlusion. Results represent mean±SEM, n=4/dose group and n=16 for the composite (“Comp”) of all I-1-treated groups combined. Pre-treatment slopes of lesion growth were determined for each subject from 0.5-1.8 hours post-occlusion. Initial post-treatment slopes were determined from 1.8-6.0 hours post-occlusion to provide sufficient timepoints for slope determination. Unpaired t-test comparing each I-1 treatment group to vehicle group (Panels A, B) and paired t-test comparing each group to itself before and after treatment.

FIG. 2 shows percent change in penumbra volume following tMCAO and treatment with vehicle or I-1. Penumbra volume was calculated from the difference between cerebral perfusion deficit and lesion volume at a threshold of <30% contralateral cerebral blood flow from 0.5 h to 3.5 h post-occlusion. Results represent mean±SEM, n=4/dose group and n=16 for the composite of all I-1-treated groups. Unpaired t-test comparing each I-1 treatment group to vehicle group.

FIG. 3A-D show comparison of DWI lesion volume growth between vehicle- and I-1-treated subjects following tMCAO. (A) Comparison of DWI lesion size and growth between vehicle-treated and a composite of all I-1-treated subjects. (B) Comparison of DWI lesion size and growth between vehicle-treated and each I-1-treated dose group. (C) Comparison of percent inhibition of DWI lesion volume at 24 hours post-occlusion. (D) Comparison of percent inhibition of DWI lesion volume at 120 hours post-occlusion. Results represent mean±SEM, n=4/dose group and n=16 for the composite of all I-1-treated groups. Unpaired t-tests.

FIG. 4A-B show Comparison of representative DWI and HE-stained lesion images from vehicle- and I-1-treated subject. (A) Representative DWI images of lesions at 0.5 h s and 120 h post-occlusion in each vehicle- and I-1 dose group. (B) Representative HE stained brain sections 120 h post-occlusion. Shaded regions denote infarcted areas.

FIG. 5A-E show I-1 plasma and CSF pharmacokinetics in non-human primates, and relationships between DWI lesion volume inhibition and average I-1 unbound plasma concentrations, total CSF concentrations and estimated A1R/A3R brain receptor occupancy following MCAO. (A) Plasma and (B) CSF pharmacokinetics. (C) Correlation between CSF and unbound plasma concentrations. Pharmacokinetics were determined following initiation of bolus/infusion regimen and compared to a reference intravenous bolus dose. (D) Relationship between % inhibition of lesion volume at final DWI measurement (120 h) and unbound plasma concentrations (red), total CSF concentrations (blue). (E) Relationship between % inhibition of lesion volume at final DWI measurement and estimated I-1 brain receptor occupancy of adenosine A1 and A3 receptors (green). Results represent mean±SEM, n=4/dose group.

FIG. 6 Shows the change in Neurological Deficit Scores from Day 1 to 5 Following Middle Cerebral Artery Occlusion in Nonhuman Primates. Neurologic deficits were assessed using the Neurological Deficit Score (NDS). Results are presented as mean±SEM, n=4 per dose group and n=16 for the composite of all I-1-treated groups combined. Statistical analyses are unpaired t-test comparing each I-1 treatment group to vehicle group.

FIG. 7 shows that a dose of 0.02 mg/kg of R-PIA demonstrated partial reversal of the decline in ATP during ischemia in rats.

### DETAILED DESCRIPTION OF THE INVENTION

### General Description of Certain Aspects of the Invention

Most drugs are ultimately targeted to specific binding sites on receptors, ion channels, transporters, or enzymes. Recent investigations have revealed the drug occupancy levels required at some target sites in order to achieve therapeutic efficacy in humans, and on the drug or ligand occupancy levels required for the activation of biological responses in animal studies. Noninvasive methodologies such as Positron Emission Tomography (PET) and Single Photon Emission Computed Tomography (SPECT) tracer studies are especially useful because they have the sensitivity required to perform target occupancy studies in both humans and animals, allowing for the quantification of a specifically bound compound in both normal and diseased tissues. Clinical and preclinical assays of target site occupancy by drugs and new drug candidates can provide optimal dosing of the desired target site with minimal side effects, and promise to reorient drug discovery and development to the achievement of specified levels of target occupancy which can most effectively stimulate or inhibit specific receptors, ion channels, transporters and enzymes. Similarly, target occupancy measurements can improve animal research studies by optimizing efficacy at the desired target site while minimizing off-target effects.

While some general insights have emerged about the degree of in vivo target occupancy needed for the effective activation or blockade of physiological and behavioral responses by drugs, ligands and endogenous agonists, there are no studies available that allow those skilled in the art to determine the receptor occupancy (RO) % necessary to yield therapeutic efficacy at the adenosine 1 and 3 receptors (A₁R and A₃R), particularly in brain and CNS targets. Human target occupancy data (by PET and SPECT) are now available for many G protein-coupled receptors (GPCRs), for select neurotransmitter transporters, and for some enzymes and ion channels, providing valuable information on the target occupancy required for efficacy at precedented targets. Due to the multiple variables that influence the RO % needed for therapeutic efficacy, earlier studies must be extended to build a rationale for predicting clinical efficacy for novel chemical entities acting at both precedented and unprecedented targets such as A₁Rs and A₃Rs.

As described in Grimwood et al., Pharmacology & Therapeutics 122 (2009) 281-301, which is hereby incorporated by reference in its entirety, the required target occupancy is dependent upon the molecular class of both target and ligand and appears to be similar for both patient therapy and human or animal physiology. In the case of antagonists, approximately 60-90% target occupancy is required for G protein-coupled receptors, neurotransmitter transporters, and ligand-gated ion channels. In contrast, the RO % (receptor occupancy %) required for effective doses of agonists can range more widely, dependent upon the intrinsic activity of the agonist, the receptor or ion channel reserve of the target site, and the response that is measured. Low efficacy agonists generally requiring high degrees of occupancy while high efficacy agonists generally require low degrees of occupancy. Target desensitization, competition by endogenous ligands, and regional target differences all influence target occupancy requirements. Measurements of target occupancy can help assure proper dosing and targeting of compounds in preclinical and clinical drug development as well as in basic research. Target occupancy generalizations can be especially important in establishing initial dosing recommendations for the many new drug targets provided by genomic and proteomic initiatives, where little data is available on their functional responses.

For example, initial dose-ranging studies of early clinical trials often require large group sizes to obtain statistically significant results using symptom evaluations as the primary outcome. These early, dose ranging clinical trial group sizes may be reduced, and development timelines shortened, if a change can be made from primary outcome measures to a surrogate marker defined by the target occupancy level needed to affect a preclinical disease model or to activate a preclinical biological response. In addition, many clinical trials have ended with no evidence of efficacy; even worse, many also end without knowing whether adequate levels of drug ever reached the intended target site. As provided by the present invention, target occupancy-guided methods provide evidence that the target site was occupied by the drug candidate to the intended extent, and thus that an adequate test of its therapeutic potential was performed.

Improved guidance of both discovery and development processes by target occupancy data could significantly shorten and improve new drug development. Molecular target occupancy data also provide important insights into physiological activation by endogenous agonists, and into quantitative requirements for ligand dosing in basic animal research. In vivo target occupancy measurements can also help answer critical questions about brain activation, such as the effects of receptor reserve (spare receptors), about regional differences in target site responses, about the effects of disease processes and disease progression on receptors, about possible differences between clinical responders and non-responders in the degree of target occupancy, and about the degree of receptor occupancy by endogenous agonists in animals and humans.

Integration of target occupancy approaches throughout the process of drug discovery and development, and in basic animal research, holds great promise for improving dose selection and optimizing on-target vs. off-target effects in both types of studies. A unified focus on target occupancy throughout drug discovery and development may also help speed new drug discovery, especially for difficult-to-treat diseases in the brain and CNS, where challenges exist in selectively delivering appropriate levels of drugs to their intended cellular and molecular target sites.

Accordingly, the present invention provides methods of evaluating the effectiveness of a small molecule drug candidate, selecting clinical doses, improving efficacy, and of treating various injuries, diseases, disorders, and conditions. Such drug candidates include the compounds described herein. In some embodiments, compounds for use in a provided method are A₁R and/or A₃R agonists.

In one aspect, the present invention provides a method of treating an injury, disease, or disorder such as a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of a compound described herein, or a pharmaceutically acceptable salt thereof or composition comprising the same, effective to reach 0.01-40% receptor occupancy (% RO) at brain or CNS A₁ receptors (A₁R) and/or A₃ receptors (A₃R) for a sufficient period of time to treat the condition.

In another aspect, the present invention provides a method of screening an A₁R, A₃R, or dual A₁R/A₃R agonist for efficacy in treating an injury, disease, or disorder such as traumatic brain injury (TBI), stroke, a neurodegenerative condition, a heart or cardiovascular disease, and a condition associated with TBI, stroke, or the neurodegenerative condition, comprising administering to a subject in need thereof an amount of the A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R).

In some embodiments, the A₁R, A₃R, or dual A₁R/A₃R agonist is effective at treating the injury, disease, or disorder if it reaches 0.01-40% RO at brain or CNS AIR and/or A₃R. In some embodiments, the A₁R, A₃R, or dual A₁R/A₃R agonist is effective at treating the injury, disease, or disorder if it reaches 1-15% RO at brain or CNS AIR and/or A₃R. In some embodiments, the agonist is a dual A₁R/A₃R agonist and is effective at treating the injury, disease, or disorder if it reaches about 1-15% RO at brain or CNS AIR and/or A₃R.

In another aspect, the present invention provides a method of determining an effective dose for treating an injury, disease, or disorder such as a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of the A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R).

In some embodiments, the effective dose of the A₁R, A₃R, or dual A₁R/A₃R agonist treats the injury, disease, or disorder if it reaches 0.01-40% RO at brain or CNS AIR and/or A₃R. In some embodiments, the effective dose of the A₁R, A₃R, or dual A₁R/A₃R agonist treats the injury, disease, or disorder if it reaches 1-15% RO at brain or CNS AIR and/or A₃R. In some embodiments, the agonist is a dual A₁R/A₃R agonist and the effective dose for treating the injury, disease, or disorder reaches about 1-15% RO at brain or CNS AIR and/or A₃R.

In another aspect, the present invention provides a method of predicting effectiveness or predicting an effective dose for treating an injury, disease, or disorder such as a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of the A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R).

In some embodiments, the effective dose of the A₁R, A₃R, or dual A₁R/A₃R agonist treats the injury, disease, or disorder if it reaches 0.01-40% RO at brain or CNS AIR and/or A₃R. In some embodiments, the effective dose of the A₁R, A₃R, or dual A₁R/A₃R agonist treats the injury, disease, or disorder if it reaches 1-15% RO at brain or CNS AIR and/or A₃R. In some embodiments, the agonist is a dual A₁R/A₃R agonist and the effective dose for treating the injury, disease, or disorder reaches about 1-15% RO at brain or CNS AIR and/or A₃R.

In some embodiments, the subject is treated with the predicted effective dose determined by the above method.

In another aspect, the present invention provides a method of optimizing a treatment regimen for an injury, disease, or disorder such as a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of the A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R).

In some embodiments, the effective dose of the A₁R, A₃R, or dual A₁R/A₃R agonist treats the injury, disease, or disorder if it reaches 0.01-40% RO at brain or CNS AIR and/or A₃R. In some embodiments, the effective dose of the A₁R, A₃R, or dual A₁R/A₃R agonist treats the injury, disease, or disorder if it reaches 0.01-30%, e.g., 1-20%, e.g., 1-15% RO at brain or CNS AIR and/or A₃R. In some embodiments, the agonist is a dual A₁R/A₃R agonist and the effective dose for treating the injury, disease, or disorder reaches about 1-15% RO at brain or CNS AIR and/or A₃R.

In some embodiments, the method further comprises the step of increasing the dose of the agonist if it fails to reach 0.01-40% RO at brain or CNS AIR and/or A₃R; and decreasing the dose of the agonist if it reaches a % RO in excess of 40%. In some embodiments, the method further comprises treating the subject for the injury, disease, or condition based on the optimized treatment regimen determined by the method.

In some embodiments, the method determines the minimum effective dose of the A₁R and/or A₃R agonist. In some embodiments, the method determines the maximum effective dose of the A₁R and/or A₃R agonist.

In some embodiments, a disclosed method is carried out on an animal subject, e.g., mouse, rat, pig, or monkey, and the method enables determination of an effective human dose. Accordingly, in some embodiments, a disclosed method provides an effective human dose based on the effective animal dose identified in the method.

In some embodiments, the compound acts as an agonist of an A₃ adenosine receptor (A₃R). In some embodiments, the compound is a partial A₃R agonist. In some embodiments, the compound is a biased A₃R agonist. In some embodiments, the compound acts by dual agonism at an A₃ adenosine receptor and an A₁ adenosine receptor (A₁R). In some embodiments, the compound acts as an agonist of an A₁ adenosine receptor (A₁R). In some embodiments, the compound is a partial A₁R agonist. In some embodiments, the compound is a biased A₁R agonist.

In some embodiments, the compound is administered in an amount effective to reach about 0.01-30% RO in the brain of the subject. In some embodiments, the compound is administered in an amount effective to reach about 0.1-25% RO in the brain of the subject. In some embodiments, the compound is administered in an amount effective to reach about 1-15% RO in the brain of the subject. In some embodiments, the compound is administered in an amount effective to reach 0.25-40%, 0.25-35%, 0.25-25%, 0.25-20%, 0.5-20%, 0.5-18%, 0.75-18%, 0.75-16%, 0.9-16%, 0.9-15%, 1.0-15%, 1.0-14%, 1.2-14%, 1.2-13%, 1.4-13%, 1.4-12%, 1.5-12%, 1.5-11%, 1.75-11%, 1.75-10%, 2.0-10%, 2.0-9.0%, 2.5-9.0%, 2.5-8.0%, 3.0-8.0%, 3.0-7.0%, 3.5-7.0%, 3.5-6.0%, 4.0-6.0%, 5.0-30%, 6.0-30%, 7.0-30%, 8.0-30%, 9.0-30%, 10-30%, 5.0-25%, 6.0-25%, 7.0-25%, 8.0-25%, 9.0-25%, 10-25%, 5.0-20%, 6.0-20%, 7.0-20%, 8.0-20%, 9.0-20%, 10-20%, 5.0-18%, 6.0-18%, 7.0-18%, 8.0-18%, 9.0-18%, 10-18%, 5.0-15%, 6.0-15%, 7.0-15%, 8.0-15%, 9.0-15%, or 10-15%; or about 0.01%, about 0.05%, about 0.1%, about 0.2%, about 0.25%, about 0.4%, about 0.5%, about 0.75%, about 0.9%, about 1.0%, about 1.2%, about 1.4%, about 1.5%, about 1.75%, about 2.0%, about 2.25%, about 2.5%, about 2.75%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11.0%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, about 30%, about 32%, about 34%, about 36%, about 38%, or about 40% RO in the brain of the subject. In some embodiments, the foregoing % RO amounts refer to the % RO of A₁R. In some embodiments, the foregoing % RO amounts refer to the % RO of A₃R. In some embodiments, the foregoing % RO amounts refer to the total % RO of A₁R and A₃R taken together.

In some embodiments, the compound is administered in an amount effective to reach about 0.01-30% RO in the CNS of the subject. In some embodiments, the compound is administered in an amount effective to reach about 0.1-25% RO in the CNS of the subject. In some embodiments, the compound is administered in an amount effective to reach about 1-15% RO in the CNS of the subject. In some embodiments, the compound is administered in an amount effective to reach 0.25-40%, 0.25-35%, 0.25-25%, 0.25-20%, 0.5-20%, 0.5-18%, 0.75-18%, 0.75-16%, 0.9-16%, 0.9-15%, 1.0-15%, 1.0-14%, 1.2-14%, 1.2-13%, 1.4-13%, 1.4-12%, 1.5-12%, 1.5-11%, 1.75-11%, 1.75-10%, 2.0-10%, 2.0-9.0%, 2.5-9.0%, 2.5-8.0%, 3.0-8.0%, 3.0-7.0%, 3.5-7.0%, 3.5-6.0%, 4.0-6.0%, 5.0-30%, 6.0-30%, 7.0-30%, 8.0-30%, 9.0-30%, 10-30%, 5.0-25%, 6.0-25%, 7.0-25%, 8.0-25%, 9.0-25%, 10-25%, 5.0-20%, 6.0-20%, 7.0-20%, 8.0-20%, 9.0-20%, 10-20%, 5.0-18%, 6.0-18%, 7.0-18%, 8.0-18%, 9.0-18%, 10-18%, 5.0-15%, 6.0-15%, 7.0-15%, 8.0-15%, 9.0-15%, or 10-15%; or about 0.01%, about 0.05%, about 0.1%, about 0.2%, about 0.25%, about 0.4%, about 0.5%, about 0.75%, about 0.9%, about 1.0%, about 1.2%, about 1.4%, about 1.5%, about 1.75%, about 2.0%, about 20.25%, about 2.5%, about 20.75%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11.0%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, about 30%, about 32%, about 34%, about 36%, about 38%, or about 40% RO in the CNS of the subject. In some embodiments, the foregoing % RO amounts refer to the % RO of A₁R. In some embodiments, the foregoing % RO amounts refer to the % RO of A₃R. In some embodiments, the foregoing % RO amounts refer to the total % RO of A₁R and A₃R taken together.

In some embodiments, the compound produces a higher % RO at the A₁R than at the A₃R. In some embodiments, the compound produces a lower % RO at the A₁R than at the A₃R. In some embodiments, the compound produces about the same % RO at the A₁R and at the A₃R.

In some embodiments, the ratio of % RO of the A₁R to the A₃R is about 1:50, 1:40, 1:30, 1:20, 1:10, 1:5, 1:2.5, 1:1.5, 1:1, 50:1, 40:1, 30:1, 20:1, 10:1, 5:1, 2.5:1, or 1.5:1.

In some embodiments, the compound is administered in an amount effective to reach about 0.01-40% RO at the A₁R and about 0.01-40% RO at the A₃R in the brain or CNS of the subject. In some embodiments, the compound is administered in an amount effective to reach about 0.01-30% RO at the A₁R and about 0.01-30% RO at the A₃R in the brain or CNS of the subject. In some embodiments, the compound is administered in an amount effective to reach about 0.1-25% RO at the A₁R and about 0.01-25% RO at the A₃R in the brain or CNS of the subject. In some embodiments, the compound is administered in an amount effective to reach about 1.0-15% RO at the A₁R and about 1.0-15% RO at the A₃R in the brain or CNS of the subject. In some embodiments, the compound is administered in an amount effective to reach 0.1-10% RO at the A₁R and about 1.0-20% RO at the A₃R in the brain or CNS of the subject. In some embodiments, the compound is administered in an amount effective to reach 1.0-20% RO at the A₁R and about 0.1-10% RO at the A₃R in the brain or CNS of the subject.

In some embodiments, the compound is administered in an amount effective to reach about 0.01%, about 0.05%, about 0.1%, about 0.2%, about 0.25%, about 0.4%, about 0.5%, about 0.75%, about 0.9%, about 1.0%, about 1.2%, about 1.4%, about 1.5%, about 1.75%, about 2.0%, about 2.25%, about 2.5%, about 2.75%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11.0%, about 11.5%, or about 12% RO at the A₁R and about 10%, about 10.5%, about 11.0%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% RO at the A₃R in the brain or CNS of the subject. In some embodiments, the compound is administered in an amount effective to reach about 10%, about 10.5%, about 11.0%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, or about 30% RO at the A₁R and about 0.01%, about 0.05%, about 0.1%, about 0.2%, about 0.25%, about 0.4%, about 0.5%, about 0.75%, about 0.9%, about 1.0%, about 1.2%, about 1.4%, about 1.5%, about 1.75%, about 2.0%, about 2.25%, about 2.5%, about 2.75%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11.0%, about 11.5%, or about 12% RO at the A₃R in the brain or CNS of the subject.

In some embodiments, the injury, disease, or disorder is traumatic brain injury (TBI), concussion, stroke (e.g., acute ischemic stroke (AIS)), partial or total spinal cord transection, malnutrition, toxic neuropathies, meningoencephalopathies, neurodegeneration caused by a genetic disorder, age-related neurodegeneration, vascular disease, Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), cardiovascular disease, autoimmune diseases, allergic diseases, transplant rejection, graft-versus-host disease, intraocular hypertension, glaucoma, odor sensitivity, an olfactory disorder, type 2 diabetes, pain control, respiratory diseases, deficits in CNS function, deficits in learning, deficits in cognition, otic disorders, Meniere's disease, endolymphatic hydrops, progressive hearing loss, dizziness, vertigo, tinnitus, collateral brain damage associated with radiation cancer therapy, migraine treatment, sleep disorders in the elderly, epilepsy, schizophrenia, symptoms experienced by recovering alcoholics, damage to neurons or nerves of the peripheral nervous system during surgery, gastrointestinal conditions, pain mediated by the CNS, migraine, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties, vertigo, dysarthria, dysphagia, ocular abnormalities or disorientation, addiction, or an addictive disorder.

In some embodiments, the injury, disease, or disorder is stroke.

In some embodiments, the stroke is selected from ischemic stroke, hemorrhagic stroke, subarachnoid hemorrhage, cerebral vasospasm, and transient ischemic attacks (TIA). In some embodiments, the stroke is ischemic, e.g., an acute ischemic stroke (AIS). In some embodiments, the stroke is hemorrhagic.

In some embodiments, the compound is administered within 8, 4, 2, or 1 hours of the stroke. In some embodiments, the compound is administered for at least the first 1-72 hours following the stroke.

In some embodiments, the present invention provides a method of treating a brain or central nervous system (CNS) injury or condition selected from traumatic brain injury (TBI) or stroke, comprising administering to a patient in need thereof an effective amount of a compound described herein, or a pharmaceutically acceptable salt thereof or composition comprising the same.

In some embodiments, the injury, disease, or disorder is TBI.

In some embodiments, the TBI is selected from concussion, whiplash, automobile accident, blast injury, combat-related injury, or a mild, moderate or severe blow to the head.

In some embodiments, neuroprotection or neurorestoration is increased in the patient as compared with an untreated patient.

In some embodiments, the injury, disease, or disorder is a neurodegenerative disease selected from Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), or a neurodegenerative condition caused by a virus, alcoholism, tumor, toxin, or repetitive brain injuries.

In some embodiments, the injury, disease, or condition is AD or ALS.

In some embodiments, the injury, disease, or disorder is a heart or cardiovascular disease selected from cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, and atherosclerosis.

In some embodiments, the heart or cardiovascular disease is cardiac ischemia or myocardial infarction.

In some embodiments, the compound or composition is administered chronically to treat stroke, cardiac ischemia, or myocardial infarction during the time period after the injury has occurred as it resolves.

In some embodiments, the compound or composition is administered within 24 hours of the TBI or stroke.

In some embodiments, the compound or composition is administered within 4 or 8 hours of the TBI or stroke.

In some embodiments, the compound or composition is administered at least during the first 4-48 hours following the TBI or stroke.

In some embodiments, the condition associated with a brain injury or a neurodegenerative condition is selected from epilepsy, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties or deficit in cognition, deficit in CNS function, deficit in learning, vertigo, dysarthria, dysphagia, ocular abnormalities, or disorientation.

In some embodiments, the compound or pharmaceutically acceptable salt thereof is administered orally, intravenously, or parenterally. In some embodiments, the compound or pharmaceutically acceptable salt thereof is administered orally. In some embodiments, the compound or pharmaceutically acceptable salt thereof is administered intravenously. In some embodiments, the compound or pharmaceutically acceptable salt thereof is administered parenterally. In some embodiments, the compound or pharmaceutically acceptable salt thereof is administered as a continuous intravenous (IV) infusion. In some embodiments, the compound or pharmaceutically acceptable salt thereof is administered initially as an IV bolus, followed by continuous IV infusion, e.g., to maintain a desired plasma concentration. In some embodiments, the compound or pharmaceutically acceptable salt thereof is administered as a slow bolus/rapid infusion. In some embodiments, the slow bolus/rapid infusion comprises IV administration over an about 5-60 minute period, e.g., a 5-30, 5-20, 10-20, or about 10 minute period.

In some embodiments, the compound is I-1, or a pharmaceutically acceptable salt thereof. In some embodiments, the compound is one of those described in Table 1 below, or a pharmaceutically acceptable salt thereof. In some embodiments, the compound is I-25 or a pharmaceutically acceptable salt thereof.

In another aspect, the present invention provides a method of treating stroke, comprising administering to a subject in need thereof an amount of the following compound:

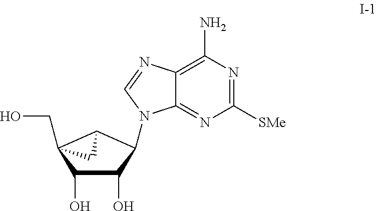

or a pharmaceutically acceptable salt thereof or composition comprising the same, effective to reach 0.01-40% receptor occupancy (% RO) at brain or CNS A₁ receptors (A₁R) and/or A₃ receptors (A₃R) for a sufficient period of time to treat the stroke.

In some embodiments, the stroke is selected from ischemic stroke, hemorrhagic stroke, subarachnoid hemorrhage, cerebral vasospasm, and transient ischemic attacks (TIA).

In some embodiments, the stroke is ischemic, e.g., an acute ischemic stroke (AIS).

In some embodiments, the stroke is hemorrhagic.

In some embodiments, the compound is administered within 48 hours of the stroke. In some embodiments, the compound is administered within 24 hours of the stroke. In some embodiments, the compound is administered within 16 hours of the stroke. In some embodiments, the compound is administered within 8, 4, 2, or 1 hours of the stroke.

In some embodiments, the compound is administered in an amount effective to reach about 0.1-25% RO at the A₁R and about 0.01-25% RO at the A₃R in the brain or CNS of the subject. In some embodiments, the compound is administered in an amount effective to reach about 1.0-15% RO at the A₁R and about 1.0-15% RO at the A₃R in the brain or CNS of the subject. In some embodiments, the compound is administered in an amount effective to reach 0.1-10% RO at the A₁R and about 1.0-20% RO at the A₃R in the brain or CNS of the subject. In some embodiments, the compound is administered in an amount effective to reach 1.0-20% RO at the A₁R and about 0.1-10% RO at the A₃R in the brain or CNS of the subject.

In some embodiments, the compound is administered in an amount effective to reach about 0.1-25% RO at the A₁R and A₃R (taken together) in the brain of the subject. In some embodiments, the compound is administered in an amount effective to reach about 1-15% RO at the A₁R and A₃R (taken together) in the brain of the subject. In some embodiments, the compound is administered in an amount effective to reach about 5-20% RO at the A₁R and A₃R (taken together) in the brain of the subject.

In some embodiments, the compound or pharmaceutically acceptable salt thereof is administered in an amount that results in a plasma concentration of about 30±20 ng/mL. In some embodiments, the compound or pharmaceutically acceptable salt thereof is administered in an amount that results in a plasma concentration of about 190±30 ng/mL. In some embodiments, the compound or pharmaceutically acceptable salt thereof is administered in an amount that results in a plasma concentration of about 480±100 ng/mL. In some embodiments, the compound or pharmaceutically acceptable salt thereof is administered in an amount that results in a plasma concentration of about 1100±300 ng/mL. In some embodiments, the compound or pharmaceutically acceptable salt thereof is administered in an amount that results in a plasma concentration of about 2500±400 ng/mL. In some embodiments, the compound or pharmaceutically acceptable salt thereof is administered in an amount that results in a plasma concentration of about 3200±400 ng/mL.

In some embodiments, the compound or pharmaceutically acceptable salt thereof is administered in an amount that results in a pharmacokinetic result selected from:
a) a plasma concentration of about 32±11 ng/mL;
b) a plasma concentration of about 186±24 ng/mL;
c) a plasma concentration of about 483±23 ng/mL; and
d) a plasma concentration of about 1127±246 ng/mL.

In some embodiments, the compound or pharmaceutically acceptable salt thereof is administered in an amount that results in a CSF concentration of about 2.0±1.0 ng/mL. In some embodiments, the compound or pharmaceutically acceptable salt thereof is administered in an amount that results in a CSF concentration of about 9.0±3.0 ng/mL. In some embodiments, the compound or pharmaceutically acceptable salt thereof is administered in an amount that results in a CSF concentration of about 17.0±9.0 ng/mL. In some embodiments, the compound or pharmaceutically acceptable salt thereof is administered in an amount that results in a CSF concentration of about 110.0±50.0 ng/mL. In some embodiments, the compound or pharmaceutically acceptable salt thereof is administered in an amount that results in a CSF concentration of about 330.0±75.0 ng/mL. In some embodiments, the compound or pharmaceutically acceptable salt thereof is administered in an amount that results in a CSF concentration of about 50-500 ng/mL. In some embodiments, the compound or pharmaceutically acceptable salt thereof is administered in an amount that results in a CSF concentration of about 70-450 ng/mL. In some embodiments, the compound or pharmaceutically acceptable salt thereof is administered in an amount that results in a CSF concentration of about 80-400 ng/mL. In some embodiments, the compound or pharmaceutically acceptable salt thereof is administered in an amount that results in a CSF concentration of about 90-350 ng/mL. In some embodiments, the compound or pharmaceutically acceptable salt thereof is administered in an amount that results in a CSF concentration of about 100-300 ng/mL. In some embodiments, the compound or pharmaceutically acceptable salt thereof is administered in an amount that results in a CSF concentration of about 110-250 ng/mL. In some embodiments, the compound or pharmaceutically acceptable salt thereof is administered in an amount that results in a CSF concentration of about 120-200 ng/mL. In some embodiments, the compound or pharmaceutically acceptable salt thereof is administered in an amount that results in a CSF concentration of about 50-150 ng/mL. In some embodiments, the compound or pharmaceutically acceptable salt thereof is administered in an amount that results in a CSF concentration of about 10-130 ng/mL. In some embodiments, the compound or pharmaceutically acceptable salt thereof is administered in an amount that results in a CSF concentration of about 15-150, 20-140, 30-130, 40-120, 50-120, 60-110, or 70-100 ng/mL.

In some embodiments, the compound or pharmaceutically acceptable salt thereof is administered in an amount that results in a pharmacokinetic result selected from:
a) a CSF concentration of about 2.1±0.4 ng/mL;
b) a CSF concentration of about 8.8±2.6 ng/mL;
c) a CSF concentration of about 16.8±5.7 ng/mL; and
d) a CSF concentration of about 108±35 ng/mL.

In some embodiments, the compound or pharmaceutically acceptable salt thereof is administered in a dose selected from:
a) about 0.05-0.25 mg/kg per day;
b) about 0.2-0.8 mg/kg per day;
c) about 0.7-2.5 mg/kg per day; and
d) about 2.3-12.0 mg/kg per day.

In some embodiments, the compound or pharmaceutically acceptable salt thereof is administered in a dose selected from:
a) about 0.11 mg/kg per day;
b) about 0.47 mg/kg per day;
c) about 1.7 mg/kg per day; and
d) about 5.2 mg/kg per day.

In some embodiments, the compound or pharmaceutically acceptable salt thereof is administered in a dose selected from:
a) about 0.03-0.12 mg/kg per hour;
b) about 0.125-0.50 mg/kg per hour;
c) about 0.45-1.8 mg/kg per hour; and
d) about 1.4-5.0 mg/kg per hour.

In some embodiments, the compound or pharmaceutically acceptable salt thereof is administered in a dose selected from:
a) about 0.06 mg/kg per hour;
b) about 0.25 mg/kg per hour;
c) about 0.9 mg/kg per hour; and
d) about 2.8 mg/kg per hour.

In some embodiments, the compound or pharmaceutically acceptable salt thereof is administered in a dose selected from:
a) about 0.05-0.25 mg/kg in combination with continuous dosing at about 0.03-0.12 mg/kg per hour;
b) about 0.2-0.8 mg/kg in combination with continuous dosing at about 0.125-0.50 mg/kg per hour;
c) about 0.7-2.5 mg/kg in combination with continuous dosing at about 0.45-1.8 mg/kg per hour; and
d) about 2.3-12.0 mg/kg in combination with continuous dosing at about 1.4-5.0 mg/kg per hour.

In some embodiments, the compound or pharmaceutically acceptable salt thereof is administered in a dose selected from:
a) about 0.11 mg/kg in combination with continuous dosing at about 0.06 mg/kg per hour;
b) about 0.47 mg/kg in combination with continuous dosing at about 0.25 mg/kg per hour;
c) about 1.7 mg/kg in combination with continuous dosing at about 0.9 mg/kg per hour; and
d) about 5.2 mg/kg in combination with continuous dosing at about 2.8 mg/kg per hour.

In some embodiments, the compound or pharmaceutically acceptable salt thereof is administered in a daily dose selected from:
a) about 6.5 mg;
b) about 28 mg;
c) about 102 mg; and
d) about 312 mg.

In some embodiments, the compound or pharmaceutically acceptable salt thereof is administered in a daily dose of about 25-75 mg. In some embodiments, the compound is compound I-1.

In some embodiments, the compound or pharmaceutically acceptable salt thereof is administered in a daily dose of about 1-400 mg. In some embodiments, the compound or pharmaceutically acceptable salt thereof is administered in a daily dose of about 6-300 mg. In some embodiments, the compound or pharmaceutically acceptable salt thereof is administered in a daily dose of about 10-200 mg. In some embodiments, the compound or pharmaceutically acceptable salt thereof is administered in a daily dose of about 20-150 mg. In some embodiments, the compound or pharmaceutically acceptable salt thereof is administered in a daily dose of about 25-100 mg.

In some embodiments, the compound or pharmaceutically acceptable salt thereof is administered in a daily dose of about 1-100 mg. In some embodiments, the compound or pharmaceutically acceptable salt thereof is administered in a daily dose of about 1-80 mg. In some embodiments, the compound or pharmaceutically acceptable salt thereof is administered in a daily dose of about 3-70 mg. In some embodiments, the compound or pharmaceutically acceptable salt thereof is administered in a daily dose of about 5-60 mg. In some embodiments, the compound or pharmaceutically acceptable salt thereof is administered in a daily dose of about 10-50 mg. In some embodiments, the compound or pharmaceutically acceptable salt thereof is administered in a daily dose of about 15-40 mg. In some embodiments, the compound or pharmaceutically acceptable salt thereof is administered in a daily dose of about 20-40 mg.

In some embodiments, the compound or pharmaceutically acceptable salt thereof is administered in a daily dose of about 100-800 mg. In some embodiments, the compound or pharmaceutically acceptable salt thereof is administered in a daily dose of about 200-400 mg. In some embodiments, the compound or pharmaceutically acceptable salt thereof is administered in a daily dose of about 75-250 mg. In some embodiments, the compound or pharmaceutically acceptable salt thereof is administered in a daily dose of about 50-150 mg.

In some embodiments, the compound or pharmaceutically acceptable salt thereof is administered in a daily dose of about 1 mg, 3 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 425 mg, 450 mg, 475 mg, 500 mg, 525 mg, 550 mg, 575 mg, or 600 mg.

In some embodiments, the subject is a mammal. In some embodiments, the subject is a mouse, rat, pig, or primate. In some embodiments, the subject is a human.

In some embodiments, the compound is represented by Formula I:

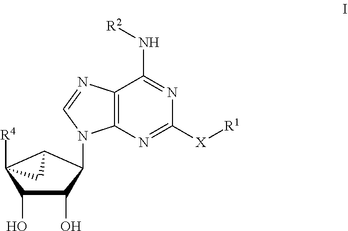

or a pharmaceutically acceptable salt thereof, wherein:
R¹ is C₁₋₈ alkyl, —(C₁₋₄ alkylene)-Ar, —(C₁₋₄ alkylene)-Cy, C₂₋₈ alkenyl, —(C₂₋₄ alkenylene)-Ar, —(C₂-4 alkenylene)-Cy, C₂₋₈ alkynyl, —(C₂₋₄ alkynylene)-Ar, —(C₂₋₄ alkynylene)-Cy, phenyl, Cy, or a 5-6 membered monocyclic heteroaromatic ring having 1, 2, 3, or 4 heteroatoms independently selected from nitrogen, oxygen, and sulfur; each of which is substituted with n instances of R³; or R¹ is a halogen when X is a covalent bond;
Ar is phenyl or a 5-6 membered monocyclic heteroaromatic ring having 1, 2, 3, or 4 heteroatoms independently selected from nitrogen, oxygen, and sulfur;
Cy is a 3-8 membered saturated or partially unsaturated monocyclic carbocyclic ring, a 7-12 membered saturated or partially unsaturated bicyclic carbocyclic ring, or a 3-6-membered saturated or partially unsaturated monocyclic heterocyclic ring having 1 or 2 heteroatoms independently selected from nitrogen, oxygen, and sulfur;
R² is hydrogen, C₁₋₄ alkyl, —(C₁₋₄ alkylene)-Ar optionally substituted with 1, 2, or 3 groups independently selected from halogen and C₁₋₄ alkyl, —(C₁₋₄ alkylene)-Cy optionally substituted with 1, 2, or 3 groups independently selected from halogen and C₁₋₄ alkyl, or C₃₋₅ cycloalkyl; wherein said C₁₋₄ alkyl and C₃₋₅ cycloalkyl are optionally substituted with 1, 2, or 3 deuterium or halogen atoms;
each R³ is independently deuterium, halogen, —CN, —O—(C₁₋₄ alkyl), —OH, —S—(C₁₋₄ alkyl), or —SH;
R⁴ is —CH₂OH or —C(O)NHR⁵;
R⁵ is H or C₁₋₄ alkyl;
X is a covalent bond, S, or O; and
n is 0, 1, 2, or 3.

The definitions of variables in Formula I above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

As defined generally above, R¹ is C₁₋₈ alkyl, —(C₁₋₄ alkylene)-Ar, —(C₁₋₄ alkylene)-Cy, C₂₋₈ alkenyl, —(C₂₋₄ alkenylene)-Ar, —(C₂₋₄ alkenylene)-Cy, C₂₋₈ alkynyl, —(C₂₋₄ alkynylene)-Ar, —(C₂₋₄ alkynylene)-Cy, phenyl, Cy, or a 5-6 membered monocyclic heteroaromatic ring having 1, 2, 3, or 4 heteroatoms independently selected from nitrogen, oxygen, and sulfur; each of which is substituted with n instances of R³; or R¹ is a halogen when X is a covalent bond.

In some embodiments, R¹ is C₁₋₈ alkyl substituted with n instances of R³. In some embodiments, R¹ is —(C₁₋₄ alkylene)-Ar substituted with n instances of R³. In some embodiments, R¹ is —(C₁₋₄ alkylene)-Cy substituted with n instances of R³. In some embodiments, R¹ is C₂₋₈ alkenyl substituted with n instances of R³. In some embodiments, R¹ is —(C₂₋₄ alkenylene)-Ar substituted with n instances of R³. In some embodiments, R¹ is —(C₂₋₄ alkenylene)-Cy substituted with n instances of R³. In some embodiments, R¹ is C₂₋₈ alkynyl substituted with n instances of R³. In some embodiments, R¹ is —(C₂₋₄ alkynylene)-Ar substituted with n instances of R³. In some embodiments, R¹ is —(C₂₋₄ alkynylene)-Cy substituted with n instances of R³. In some embodiments, R¹ is phenyl substituted with n instances of R³. In some embodiments, R¹ is Cy substituted with n instances of R³. In some embodiments, R¹ is a 5-6 membered monocyclic heteroaromatic ring having 1, 2, 3, or 4 heteroatoms independently selected from nitrogen, oxygen, and sulfur; wherein said ring is substituted with n instances of R³. In some embodiments, X is a covalent bond and R¹ is a halogen.

In some embodiments, R¹ is C₁₋₈ alkyl, —(C₁₋₄ alkylene)-phenyl, —(C₁₋₄ alkylene)-(C₃₋₈ cycloalkyl), C₂₋₈ alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³.

In some embodiments, R¹ is C₁₋₈ alkyl, —(C₁₋₄ alkylene)-phenyl, —(C₁₋₄ alkylene)-(C₃₋₈ cycloalkyl), C₂₋₈ alkenyl, or C₂₋₈ alkynyl; each of which is substituted with n instances of R³. In some embodiments, R¹ is C₁₋₈ alkyl, —(C₁₋₂ alkylene)-phenyl, or —(C₁₋₂ alkylene)-(C₃₋₅ cycloalkyl); each of which is substituted with n instances of R³. In some embodiments, R¹ is C₁₋₈ alkyl, —(C₁₋₂ alkylene)-phenyl, or —(C₁₋₂ alkylene)-(C₃₋₅ cycloalkyl). In some embodiments, R¹ is —(C₁₋₂ alkylene)-phenyl or —(C₁₋₂ alkylene)-(C₃₋₅ cycloalkyl).

In some embodiments, R¹ is C₁₋₈ alkyl, —(C₁₋₂ alkylene)-phenyl, —(C₁₋₂ alkylene)-(C₃₋₅ cycloalkyl), or C₃₋₈ cycloalkyl; each of which is substituted with n instances of R³.

In some embodiments, R¹ is C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³.

In some embodiments, R¹ is C₁₋₆ alkyl substituted with n instances of R³. In some embodiments, R¹ is C₁₋₄ alkyl substituted with n instances of R³. In some embodiments, R¹ is C₃₋₈ alkyl substituted with n instances of R³. In some embodiments, R¹ is C₃₋₆ alkyl substituted with n instances of R³. In some embodiments, R¹ is C₃₋₄ alkyl substituted with n instances of R³. In some embodiments, R¹ is (i) C₁₋₂ alkyl substituted with 1, 2, or 3 instances of R³, or (ii) C₃₋₈ alkyl substituted with n instances of R³. In some embodiments, R¹ is C₁₋₈ alkyl substituted with 1, 2, or 3 instances of R³. In some embodiments, R¹ is —(C₁₋₄ alkylene)-phenyl substituted with n instances of R³. In some embodiments, R¹ is —(C₁₋₂ alkylene)-phenyl substituted with n instances of R³. In some embodiments, R¹ is —(C₁₋₄ alkylene)-(C₃₋₈ cycloalkyl) substituted with n instances of R³. In some embodiments, R¹ is —(C₁₋₂ alkylene)-(C₃₋₅ cycloalkyl) substituted with n instances of R³. In some embodiments, R¹ is C₃₋₈ cycloalkyl substituted with n instances of R³. In some embodiments, R¹ is C₃₋₆ cycloalkyl substituted with n instances of R³.

In some embodiments, R¹ is C₁₋₈ alkyl. In some embodiments, R¹ is C₁₋₆ alkyl. In some embodiments, R¹ is C₁₋₄ alkyl. In some embodiments, R¹ is methyl or ethyl. In some embodiments, R¹ is methyl. In some embodiments, R¹ is ethyl. In some embodiments, R¹ is C₃₋₈ alkyl. In some embodiments, R¹ is C₃₋₆ alkyl. In some embodiments, R¹ is C₃₋₄ alkyl. In some embodiments, R¹ is —(C₁₋₄ alkylene)-phenyl. In some embodiments, R¹ is —(C₁₋₂ alkylene)-phenyl. In some embodiments, R¹ is —(C₁₋₄ alkylene)-(C₃.s cycloalkyl). In some embodiments, R¹ is —(C₁₋₂ alkylene)-(C₃₋₅ cycloalkyl). In some embodiments, R¹ is C₂₋₈ alkenyl. In some embodiments, R¹ is C₂₋₈ alkynyl. In some embodiments, R¹ is C₃₋₈ cycloalkyl. In some embodiments, R¹ is C₃₋₆ cycloalkyl. In some embodiments, R¹ is phenyl. In some embodiments, R¹ is a 5-6 membered monocyclic heteroaromatic ring having 1-4 heteroatoms independently selected from nitrogen, oxygen, and sulfur.

In some embodiments, R¹ is (i) C₁₋₂ alkyl substituted with 1, 2, or 3 instances of R³, or (ii) C₃₋₈ alkyl, —(C₁₋₄ alkylene)-phenyl, —(C₁₋₄ alkylene)-(C₃₋₈ cycloalkyl), C₂₋₈ alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³. In some embodiments, R¹ is (i) C₁₋₈ alkyl substituted with 1, 2, or 3 instances of R³, or (ii)—(C₁₋₄ alkylene)-phenyl, —(C₁₋₄ alkylene)-(C₃.s cycloalkyl), C₂₋ₛ alkenyl, C₂-8 alkynyl, C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³. In some embodiments, R¹ is (i) C₁₋₂ alkyl substituted with 1, 2, or 3 instances of R³, or (ii) C₃₋₈ alkyl, —(C₁₋₂ alkylene)-phenyl, —(C₁₋₂ alkylene)-(C₃₋₅ cycloalkyl), or C₃₋₈ cycloalkyl; each of which is substituted with n instances of R³. In some embodiments, R¹ is C₃₋₈ alkyl, —(C₁₋₄ alkylene)-phenyl, —(C₁₋₄ alkylene)-(C₃.s cycloalkyl), C_{2-g} alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³. In some embodiments, R¹ is —(C₁₋₄ alkylene)-phenyl, —(C₁₋₄ alkylene)-(C₃₋₈ cycloalkyl), C₂₋₈ alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³.

In some embodiments, X is a covalent bond and R¹ is a halogen selected from F or Cl. In some embodiments, R¹ is F. In some embodiments, R¹ is Cl.

In some embodiments, R¹ is selected from those depicted in Table 1, below.

As defined generally above, Ar is phenyl or a 5-6 membered monocyclic heteroaromatic ring having 1, 2, 3, or 4 heteroatoms independently selected from nitrogen, oxygen, and sulfur.

In some embodiments, Ar is phenyl. In some embodiments, Ar is a 5-6 membered monocyclic heteroaromatic ring having 1, 2, 3, or 4 heteroatoms independently selected from nitrogen, oxygen, and sulfur.

In some embodiments, Ar is selected from those depicted in Table 1, below.

As defined generally above, Cy is a 3-8 membered saturated or partially unsaturated monocyclic carbocyclic ring, a 7-12 membered saturated or partially unsaturated bicyclic carbocyclic ring, or a 3-6-membered saturated or partially unsaturated monocyclic heterocyclic ring having 1 or 2 heteroatoms independently selected from nitrogen, oxygen, and sulfur.

In some embodiments, Cy is a 3-8 membered saturated or partially unsaturated monocyclic carbocyclic ring. In some embodiments, Cy is a 3-8 membered saturated monocyclic carbocyclic ring. In some embodiments, Cy is a 3-6 membered saturated monocyclic carbocyclic ring. In some embodiments, Cy is a 7-12 membered saturated or partially unsaturated bicyclic carbocyclic ring. In some embodiments, Cy is a 3-6-membered saturated or partially unsaturated monocyclic heterocyclic ring having 1 or 2 heteroatoms independently selected from nitrogen, oxygen, and sulfur.

In some embodiments, Cy is selected from those depicted in Table 1, below.

As defined generally above, R² is hydrogen, C₁₋₄ alkyl, —(C₁₋₄ alkylene)-Ar optionally substituted with 1, 2, or 3 groups independently selected from halogen and C₁₋₄ alkyl, —(C₁₋₄ alkylene)-Cy optionally substituted with 1, 2, or 3 groups independently selected from halogen and C₁₋₄ alkyl, or C₃₋₅ cycloalkyl; wherein said C₁₋₄ alkyl and C₃₋₅ cycloalkyl are optionally substituted with 1, 2, or 3 deuterium or halogen atoms.

In some embodiments, R² is —(C₁₋₄ alkylene)-Ar optionally substituted with 1, 2, or 3 groups independently selected from halogen and C₁₋₄ alkyl. In some embodiments, R² is —(C₁₋₄ alkylene)-Cy optionally substituted with 1, 2, or 3 groups independently selected from halogen and C₁₋₄ alkyl. In some embodiments, R² is C₁₋₄ alkyl or C₃₋₅ cycloalkyl; each of which is optionally substituted with 1, 2, or 3 deuterium or halogen atoms. In some embodiments, R² is C₁₋₄ alkyl optionally substituted with 1, 2, or 3 deuterium or halogen atoms. In some embodiments, R² is C₁₋₄ alkyl substituted with 1, 2, or 3 deuterium or halogen atoms. In some embodiments, R² is C₃₋₅ cycloalkyl optionally substituted with 1, 2, or 3 deuterium or halogen atoms. In some embodiments, R² is C₃₋₅ cycloalkyl substituted with 1, 2, or 3 deuterium or halogen atoms.

In some embodiments, R² is hydrogen, C₁₋₄ alkyl, or C₃₋₅ cycloalkyl. In some embodiments, R² is hydrogen or C₁₋₄ alkyl. In some embodiments, R² is C₁₋₄ alkyl or C₃₋₅ cycloalkyl. In some embodiments, R² is hydrogen. In some embodiments, R² is C₁₋₄ alkyl. In some embodiments, R² is methyl or ethyl. In some embodiments, R² is methyl. In some embodiments, R² is C₃₋₅ cycloalkyl. In some embodiments, R² is cyclopropyl.

In some embodiments, R² is selected from those depicted in Table 1, below.

As defined generally above, each R³ is independently deuterium, halogen, —CN, —O—(C₁. 4 alkyl), —OH, —S—(C₁₋₄ alkyl), or —SH.

In some embodiments, each R³ is independently halogen, —O—(C₁₋₄ alkyl), —OH, —S—(C₁₋₄ alkyl), or —SH. In some embodiments, each R³ is deuterium. In some embodiments, each R³ is independently halogen. In some embodiments, each R³ is independently fluoro or chloro. In some embodiments, R³ is fluoro. In some embodiments, each R³ is —CN. In some embodiments, each R³ is independently —O—(C₁₋₄ alkyl) or —OH. In some embodiments, each R³ is independently —O—(C₁₋₄ alkyl). In some embodiments, R³ is —OH. In some embodiments, each R³ is independently —S—(C₁₋₄ alkyl) or —SH. In some embodiments, each R³ is independently —S—(C₁₋₄ alkyl). In some embodiments, R³ is —SH.

In some embodiments, R³ is selected from those depicted in Table 1, below.

As defined generally above, R⁴ is —CH₂OH or —C(O)NHR⁵.

In some embodiments, R⁴ is —CH₂OH. In some embodiments, R⁴ is —C(O)NHR⁵.

In some embodiments, R⁴ is —C(O)NH₂. In some embodiments, R⁴ is —C(O)NHMe. In some embodiments, R⁴ is —C(O)NHEt.

In some embodiments, R⁴ is selected from those depicted in Table 1, below.

As defined generally above, R⁵ is H or C₁₋₄ alkyl.

In some embodiments, R⁵ is selected from those depicted in Table 1, below.

In some embodiments, R⁵ is H. In some embodiments, R⁵ is C₁₋₄ alkyl.

As defined generally above, X is a covalent bond, S, or O. In some embodiments, X is a covalent bond. In some embodiments, X is S. In some embodiments, X is O. In some embodiments, X is a covalent bond and R¹ is halogen. In some embodiments, R¹ is selected from those depicted in Table 1, below.

As defined generally above, n is 0, 1, 2, or 3. In some embodiments, n is 0. In some embodiments, n is 1. In some embodiments, n is 2. In some embodiments, n is 3. In some embodiments, n is 0 or 1. In some embodiments, n is 1 or 2. In some embodiments, n is 2 or 3. In some embodiments, n is 0, 1, or 2. In some embodiments, n is 1, 2, or 3. In some embodiments, n is selected from those depicted in Table 1, below.

The description above describes multiple embodiments relating to compounds of Formula I. The patent application specifically contemplates all combinations of the embodiments.

In some embodiments, the compound is of Formula I-A:

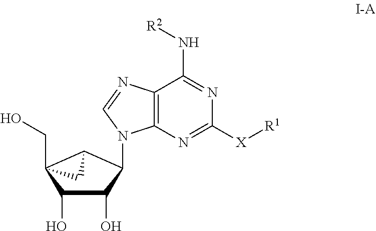

or a pharmaceutically acceptable salt thereof, wherein:
R¹ is (i) C₁₋₂ alkyl optionally substituted with 1, 2, or 3 instances of R³, or (ii) C₃₋₈ alkyl, —(C₁₋₄ alkylene)-phenyl, —(C₁₋₄ alkylene)-(C₃₋₈ cycloalkyl), C_{2-g} alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is optionally substituted with n instances of R³;
R² is hydrogen, C₁₋₄ alkyl, or C₃₋₅ cycloalkyl;
each R³ is independently halogen, —O—(C₁₋₄ alkyl), —OH, —S—(C₁₋₄ alkyl), or —SH;
X is S or O; and
n is 0, 1, 2, or 3.

In some embodiments, the compound is of Formula I-B:

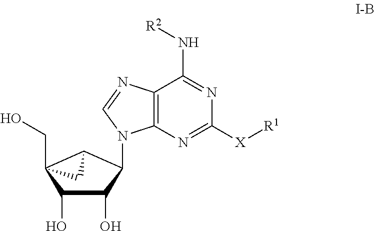

or a pharmaceutically acceptable salt thereof, wherein:
R¹ is a halogen;
R² is hydrogen, C₁₋₄ alkyl, or C₃₋₅ cycloalkyl; and
X is a covalent bond.

The definitions of variables in Formula I-A and I-B above encompass multiple chemical groups. The application contemplates embodiments where, for example, (i) the definition of a variable is a single chemical group selected from those chemical groups set forth above, (ii) the definition of a variable is a collection of two or more of the chemical groups selected from those set forth above, and (iii) the compound is defined by a combination of variables in which the variables are defined by (i) or (ii).

In certain embodiments, the compound is a compound of Formula I-A. In certain embodiments, the compound is a compound of Formula I-B.

As defined generally above, R¹ is (i) C₁₋₂ alkyl optionally substituted with 1, 2, or 3 instances of R³, or (ii) C₃₋₈ alkyl, —(C₁₋₄ alkylene)-phenyl, —(C₁₋₄ alkylene)-(C₃₋₈ cycloalkyl), C₂₋₉ alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is optionally substituted with n instances of R³.

In some embodiments, R¹ is (i) C₁₋₂ alkyl optionally substituted with 1, 2, or 3 instances of R³, or (ii) C₃₋₈ alkyl, —(C₁₋₂ alkylene)-phenyl, —(C₁₋₂ alkylene)-(C₃₋₅ cycloalkyl), or C₃₋₈ cycloalkyl; each of which is optionally substituted with n instances of R³.

In some embodiments, R¹ is C₁₋₂ alkyl substituted with 1, 2, or 3 instances of R³. In some embodiments, R¹ is C₃₋₈ alkyl, —(C₁₋₄ alkylene)-phenyl, —(C₁₋₄ alkylene)-(C₃₋₈ cycloalkyl), C₂-8 alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³.

In some embodiments, R¹ is —(C₁₋₄ alkylene)-phenyl, —(C₁₋₄ alkylene)-(C₃₋₈ cycloalkyl), C₂₋ₛ alkenyl, or C₂₋₈ alkynyl; each of which is substituted with n instances of R³. In some embodiments, R¹ is —(C₁₋₂ alkylene)-phenyl or —(C₁₋₂ alkylene)-(C₃₋₅ cycloalkyl); each of which is substituted with n instances of R³. In some embodiments, R¹ is —(C₁₋₂ alkylene)-phenyl or —(C₁₋₂ alkylene)-(C₃₋₅ cycloalkyl).

In some embodiments, R¹ is C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³.

In some embodiments, R¹ is C₃₋₈ alkyl substituted with n instances of R³. In some embodiments, R¹ is C₃₋₆ alkyl substituted with n instances of R³. In some embodiments, R¹ is C₃. 4 alkyl substituted with n instances of R³. In some embodiments, R¹ is —(C₁₋₄ alkylene)-phenyl substituted with n instances of R³. In some embodiments, R¹ is —(C₁₋₂ alkylene)-phenyl substituted with n instances of R³. In some embodiments, R¹ is —(C₁₋₄ alkylene)-(C₃.s cycloalkyl) substituted with n instances of R³. In some embodiments, R¹ is —(C₁₋₂ alkylene)-(C₃₋₅ cycloalkyl) substituted with n instances of R³. In some embodiments, R¹ is C₂₋₈ alkenyl substituted with n instances of R³. In some embodiments, R¹ is C₂₋₈ alkynyl substituted with n instances of R³. In some embodiments, R¹ is C₃₋₈ cycloalkyl substituted with n instances of R³. In some embodiments, R¹ is C₃₋₆ cycloalkyl substituted with n instances of R³. In some embodiments, R¹ is phenyl substituted with n instances of R³. In some embodiments, R¹ is a 5-6 membered monocyclic heteroaromatic ring having 1-4 heteroatoms independently selected from nitrogen, oxygen, and sulfur; wherein said ring is substituted with n instances of R³.

In some embodiments, R¹ is C₃₋₈ alkyl. In some embodiments, R¹ is C₃₋₆ alkyl. In some embodiments, R¹ is C₃₋₄ alkyl. In some embodiments, R¹ is —(C₁₋₄ alkylene)-phenyl. In some embodiments, R¹ is —(C₁₋₂ alkylene)-phenyl. In some embodiments, R¹ is —(C₁₋₄ alkylene)-(C₃₋₈ cycloalkyl). In some embodiments, R¹ is —(C₁₋₂ alkylene)-(C₃₋₅ cycloalkyl). In some embodiments, R¹ is C₂₋₈ alkenyl. In some embodiments, R¹ is C₂₋₈ alkynyl. In some embodiments, R¹ is C₃₋₈ cycloalkyl. In some embodiments, R¹ is C₃₋₆ cycloalkyl. In some embodiments, R¹ is phenyl. In some embodiments, R¹ is a 5-6 membered monocyclic heteroaromatic ring having 1-4 heteroatoms independently selected from nitrogen, oxygen, and sulfur.

In some embodiments, R¹ is (i) C₁₋₈ alkyl substituted with 1, 2, or 3 instances of R³, or (ii)—(C₁₋₄ alkylene)-phenyl, —(C₁₋₄ alkylene)-(C₃.s cycloalkyl), C₂₋₈ alkenyl, C₂₋₈ alkynyl, C₃.s cycloalkyl, phenyl, or a 5-6 membered monocyclic heteroaromatic ring having 1-4 heteroatoms independently selected from nitrogen, oxygen, and sulfur; each of which is substituted with n instances of R³. In some embodiments, R¹ is C₃₋₈ alkyl, —(C₁₋₄ alkylene)-phenyl, —(C₁₋₄ alkylene)-(C₃₋₈ cycloalkyl), C₂₋ₛ alkenyl, C₂₋₈ alkynyl, C₃₋₈ cycloalkyl, phenyl, or a 5-6 membered monocyclic heteroaromatic ring having 1-4 heteroatoms independently selected from nitrogen, oxygen, and sulfur; each of which is substituted with n instances of R³. In some embodiments, R¹ is —(C₁₋₄ alkylene)-phenyl, —(C₁₋₄ alkylene)-(C₃.s cycloalkyl), C₂₋₈ alkenyl, C₂₋₈ alkynyl, C₃.s cycloalkyl, phenyl, or a 5-6 membered monocyclic heteroaromatic ring having 1-4 heteroatoms independently selected from nitrogen, oxygen, and sulfur; each of which is substituted with n instances of R³.

In some embodiments, R¹ is selected from those depicted in Table 1, below.

As defined generally above, R² is hydrogen, C₁₋₄ alkyl, or C₃₋₅ cycloalkyl.

In some embodiments, R² is hydrogen or C₁₋₄ alkyl. In some embodiments, R² is C₁₋₄ alkyl or C₃₋₅ cycloalkyl. In some embodiments, R² is hydrogen. In some embodiments, R² is C₁. 4 alkyl. In some embodiments, R² is methyl or ethyl. In some embodiments, R² is methyl. In some embodiments, R² is C₃₋₅ cycloalkyl. In some embodiments, R² is cyclopropyl.

In some embodiments, R² is selected from those depicted in Table 1, below.

As defined generally above, each R³ is independently halogen, —O—(C₁₋₄ alkyl), —OH, —S—(C₁₋₄ alkyl), or —SH.

In some embodiments, each R³ is independently halogen. In some embodiments, each R³ is independently fluoro or chloro. In some embodiments, R³ is fluoro. In some embodiments, each R³ is independently —O—(C₁₋₄ alkyl) or —OH. In some embodiments, each R³ is independently O—(C₁₋₄ alkyl). In some embodiments, R³ is —OH. In some embodiments, each R³ is independently —S—(C₁₋₄ alkyl) or —SH. In some embodiments, each R³ is independently —S—(C₁₋₄ alkyl). In some embodiments, R³ is —SH.

In some embodiments, R³ is selected from those depicted in Table 1, below.

As defined generally above, X is S or O. In some embodiments, X is S. In some embodiments, X is O. In some embodiments, R¹ is selected from those depicted in Table 1, below.

As defined generally above, n is 0, 1, 2, or 3. In some embodiments, n is 0. In some embodiments, n is 1. In some embodiments, n is 2. In some embodiments, n is 3. In some embodiments, n is 0 or 1. In some embodiments, n is 1 or 2. In some embodiments, n is 2 or 3. In some embodiments, n is 0, 1, or 2. In some embodiments, n is 1, 2, or 3. In some embodiments, n is selected from those depicted in Table 1, below.

The description above describes multiple embodiments relating to compounds of Formula I-A. The patent application specifically contemplates all combinations of the embodiments.

In some embodiments, the compound for use in a provided method is a compound selected from one of those in Table 1, or a pharmaceutically acceptable salt thereof.

As defined generally above for Formula I-B, R¹ is a halogen. In some embodiments, R¹ is F. In some embodiments, R¹ is Cl. In some embodiments, R¹ is Br. In some embodiments, R¹ is I.

As defined generally above for Formula I-B, R² is hydrogen, C₁₋₄ alkyl, or C₃₋₅ cycloalkyl.

In some embodiments, R² is hydrogen or C₁₋₄ alkyl. In some embodiments, R² is C₁₋₄ alkyl or C₃₋₅ cycloalkyl. In some embodiments, R² is hydrogen. In some embodiments, R² is C₁. 4 alkyl. In some embodiments, R² is methyl or ethyl. In some embodiments, R² is methyl. In some embodiments, R² is C₃₋₅ cycloalkyl. In some embodiments, R² is cyclopropyl.

In some embodiments, the compound of Formula I-B is:

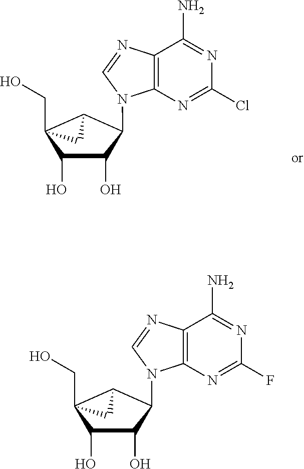

or a pharmaceutically acceptable salt thereof.

U.S. Pat. Nos. 9,789,131 and 10,765,693, the entirety of each of which is hereby incorporated herein by reference, describe certain therapeutically beneficial compounds. Such compounds include compound I-1:

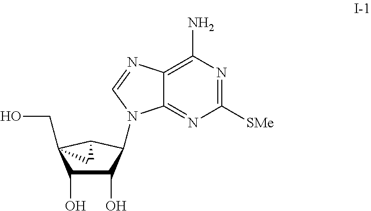

or a pharmaceutically acceptable salt thereof.

Compound I-1 is designated as MRS4322 in U.S. Pat. No. 9,789,131 and the synthesis of compound I-1 is described in detail at Example 9 of U.S. Pat. No. 9,789,131. Compound I-1 is designated as Compound A in U.S. Pat. No. 10,765,693 and its synthesis and preparation of solid forms thereof is described in detail at Example A and subsequent Examples therein.

In some embodiments, the compound is selected from one of those described in U.S. Pat. No. 9,789,131, the entirety of which is hereby incorporated by reference. In some embodiments, the compound is selected from: adenosine, ADP, 2-methylthio-ADP trisodium salt, ATP, ATP disodium salt, α,β-methylene ATP, α,β-methyleneadenosine 5′-triphosphate trisodium salt, 2-methylthioadenosine triphosphate tetrasodium salt, 2-MeSATP, BzATP triethylammonium salt, inosine, cytidine, acylated cytidines, cytidine-monophosphate (CMP), cytidine diphosphate (CDP), cytidine triphosphate (CTP), CDP-choline, CMP-choline, denufosol, denufosol tetrasodium, GTP, ITP, MRS 541, MRS 542, MRS 1760, MRS 2179, MRS 2279, MRS 2341, MRS 2365, MRS 2500, MRS 2690, MRS 2698, MRS 3558, MRS 4322, MRS 5151, MRS 5676, MRS 5678, MRS 5697, MRS 5698, MRS 5923, MRS 5930, Benzyl-NECA, IB-MECA, Cl-IB-MECA, LJ529, DPMA, CCPA, DBXRM, HEMADO, PEMADO, HENECA, PENECA, CP608,039, CP532,903, CGS21680, AR132, VT72, VT158, VT160, VT163, PSB 0474, uridine 5′-diphosphate (UDP), UDP-glucose, uridine P-thiodiphosphate (UDPRS), uridine 5′-triphosphate (UTP), uridine 7-thiophosphate (UTPTS), 2-thioUTP tetrasodium salt, UTPTS trisodium salt, uridine-5′-diphosphoglucose, diuridine triphosphate, 2-(hexylthio) (HT)-AMP, diadenosine pentaphosphate, 2′-deoxy-2′-amino-UTP, 2-thio-UTP, triacetyluridine, diacetyl/acyl uridine, uridine, suramin, dipyridamole analogs, diadenosine tetraphosphate Ap₄U, Ap₄A, INS365, INS37217, or INS48823; wherein the ribo- or 2-deoxyribo sugar of any of the foregoing compounds may be replaced with a methanocarba sugar in the North conformation; or a pharmaceutically acceptable salt thereof.

In some embodiments, 2-methylthio-ADP or a pharmaceutically acceptable salt thereof is useful in the methods of the present invention. Without wishing to be bound by theory, it is believed that 2-MeS ADP is rapidly hydrolyzed to 2-methylthioadenosine in vivo, where it is a biased agonist, partial agonist, or biased partial agonist of A₁R/or A₃R.

In some embodiments, the compound is an A₁R and/or A₃R agonist such as N⁶-benzyladenosine-5′-N-methyluronamides such as N⁶-(3-iodobenzyl)-adenosine-5′-N-methyluronamide, also known as IB-IMECA or Can-Fite CF-101, or 2-Chloro-N⁶-(3-iodobenzyl)-adenosine-5′-N-methyluronamide (also known as 2-CI-IB-MECA or Can-Fite CF-102; (N)-methanocarba nucleosides such as (1R,2R,3S,4R)-4-(2-chloro-6-((3-chlorobenzyl)amino)-9H-purin-9-yl)-2,3-di-hydroxy-N-methylbicyclo[3.1.0]hexane-1-carboxamide (also known as CF502, Can-Fite Biopharma, MA); (2S,3S,4R,5R)-3-amino-5-[6-(2,5-dichlorobenzylamino)purin-9-yl]-4-hydroxy-tetrahydrofuran-2-carboxylic acid methylamide (also known as CP532,903); (1'S,2′R,3'S,4′R,5'S)-4-(2-chloro-6-(3-chlorobenzylamino)-9H-purin-9-yl)-2,3-dihydroxy-N-methylbicyclo[3.1.0]hexane-1-carboxamide (also known as MRS3558), 2-(1-hexynyl)-N-methyladenosine; (1S,2R,3S,4R)-2,3-dihydroxy-4-(6-((3-iodobenzyl)amino)-4H-purin-9(5H)-yl)-N-methylcyclopentanecarboxamide (also known as CF101, Can-Fite); (1S,2R,3S,4R)-4-(2-chloro-6-((3-iodobenzyl)amino)-4H-purin-9(5H)-yl)-2,3-dihydroxy-N-methylcyclopentanecarboxamide (also known as CF102, Can-Fite); (1′R,2′R,3'S,4′R,5'S)-4-{2-chloro-6-[(3-iodophenylmethyl)amino]purin-9-yl-}-1-(methylaminocarbonyl)-bicyclo[3.1.0]hexane-2,3-diol (also known as MRS1898); or 2-dialkynyl derivatives of (N)-methanocarba nucleosides; or a pharmaceutically acceptable salt thereof. In some embodiments, the compound is selected from IB-MECA (also known as CF101), or Cl-IB-MECA (also known as CF102); or a pharmaceutically acceptable salt thereof. In some embodiments, the compound is selected from a (N)-methanocarba nucleoside such as those disclosed above; or a pharmaceutically acceptable salt thereof. In some embodiments, the compound is one of those disclosed in WO 2014/160502, which is hereby incorporated by reference in its entirety.

Also included are A₃R allosteric modulators which enhance the receptor activity in the presence of the native ligand, such as 2-cyclohexyl-N-(3,4-dichlorophenyl)-1H-imidazo[4,5-c]quinolin-4-amine (also known as CF602, Can-Fite). However, the above-listed A₃R agonists are by no means exclusive and other such agonists may also be used. The administration of A₃R agonists covalently bound to polymers is also contemplated. For example, A₃R agonists may be administered in the form of conjugates where an agonist is bound to a polyamidoamine (PAMAM) dendrimer.

In some embodiments, the compound is selected from:

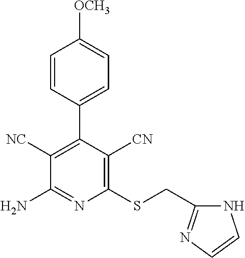 [SMILES: COc1ccc(-c2c(C#N)c(N)nc(SCc3ncc[nH]3)c2C#N)cc1]

(see Beukers M W et al., (2004) “New, non-adenosine, high-potency agonists for the human adenosine A2B receptor with an improved selectivity profile compared to the reference agonist N-ethylcarboxamidoadenosine,” J. Med. Chem. 47(15):3707-3709);

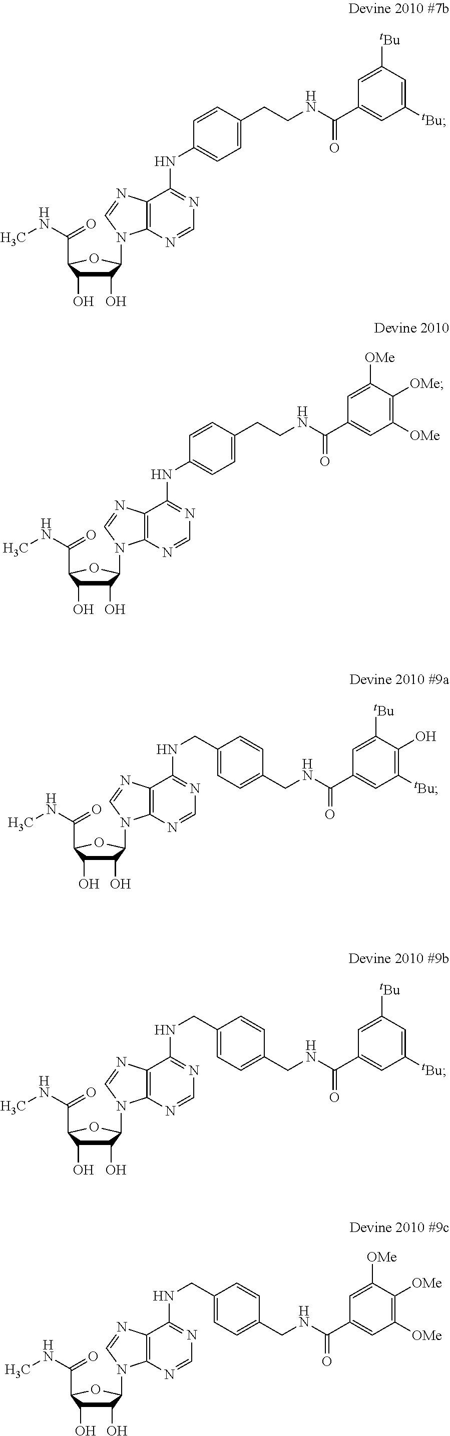

(see Devine S M et al. “Synthesis and Evaluation of new A3R agonists,” Bioorg Med Chem 18, 3078-3087. 2010; and Muller C E, Jacobson K A. “Recent Developments in adenosine receptor ligands and their potential for novel drugs,” Biochimica et Biophysica Acta 1808, 1290-1308. 2011);

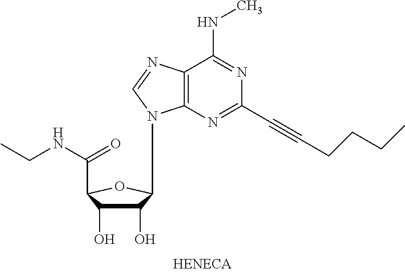

(see Ben D D et al. “Different efficacy of adenosine and NECA derivatives at the human A3 receptor: Insight into the receptor activation switch,” Biochem Pharm 87, 321-331. 2014; and Camaioni E, Di Francesco E, Vittori S, Volpini R, Cristalli G. “Adenosine receptor agonists: synthesis and biological evaluation of the diastereoisomers of 2-(3-hydroxy-3-phenyl-1-propyn-1-yl)NECA,” Bioorg Med Chem 1997; 5:2267-75);

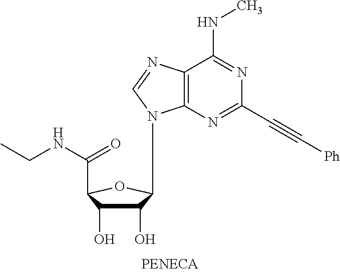

(see Klotz, K. N. “2-Substituted N-ethylcarboxamidoadenosine derivatives as high-affinity agonists at human A3 adenosine receptors,” Naunyn Schmiedebergs Arch Pharmacol. 1999 August; 360(2):103-8; and Cristalli G et al. (1995) “2-Aralkynyl and 2-heteroalkynyl derivatives of adenosine-5′-N-ethyluronamide as selective A2a adenosine receptor agonists,” J Med Chem 38:1462-1472);

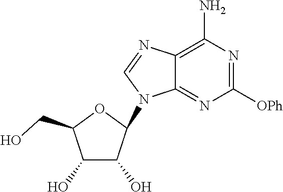 [SMILES: Nc1nc(Oc2ccccc2)nc2c1ncn2[C@@H]1O[C@H](CO)[C@@H](O)[C@H]1O]

(see Kim S et al. “3D quantitative SAR at A3R,” J Chem Inf. Model 47, 1225-1233 2007);

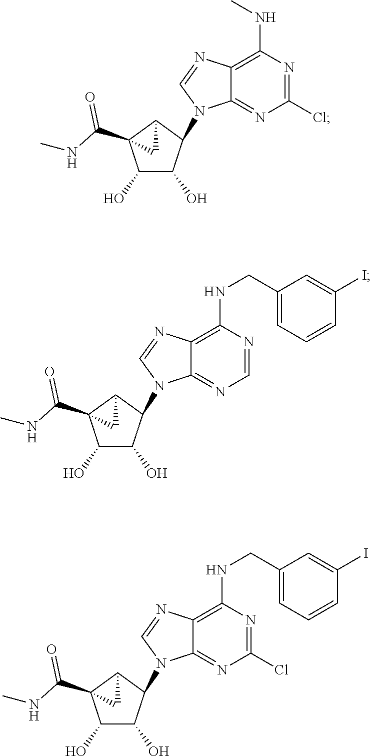 [SMILES: CNC(=O)[C@]12C[C@@H]1[C@@H](n1cnc3c(NC)nc(Cl)nc31)[C@H](O)[C@@H]2O ; CNC(=O)[C@]12C[C@@H]1[C@@H](n1cnc3c(NCc4cccc(I)c4)ncnc31)[C@H](O)[C@@H]2O ; CNC(=O)[C@]12C[C@@H]1[C@@H](n1cnc3c(NCc4cccc(I)c4)nc(Cl)nc31)[C@H](O)[C@@H]2O]

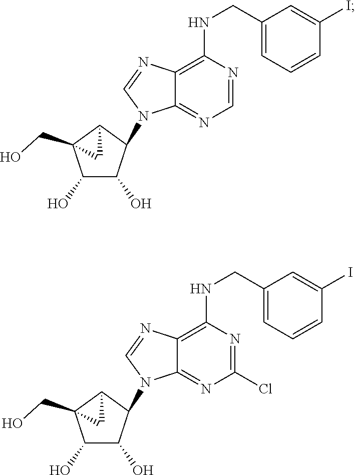 [SMILES: OC[C@]12C[C@@H]1[C@@H](n1cnc3c(NCc4cccc(I)c4)ncnc31)[C@H](O)[C@@H]2O ; OC[C@]12C[C@@H]1[C@@H](n1cnc3c(NCc4cccc(I)c4)nc(Cl)nc31)[C@H](O)[C@@H]2O]

### (see Lee, K. et al. “Ring-Constrained (N)-Methanocarba Nucleosides as Adenosine Receptor Agonists,” Bioorg Med Chem Lett 2001, 11, 1333-1337);

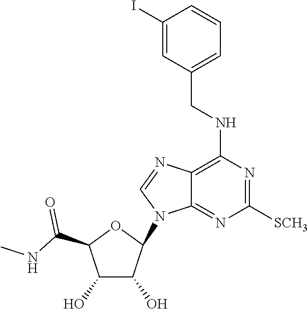 [SMILES: CNC(=O)[C@H]1O[C@@H](n2cnc3c(NCc4cccc(I)c4)nc(SC)nc32)[C@H](O)[C@@H]1O]

(see Kenneth A. Jacobson et al. Chapter 6. A3 Adenosine Receptor Agonists: History and Future Perspectives pp 96-97. Book—Springer: A3 Adenosine Receptors from Cell Biology to Pharmacology and Therapeutics, 2009);

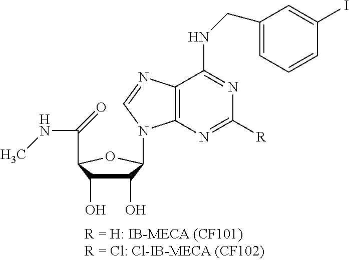

see Muller C E, Jacobson K A, “Recent Developments in adenosine receptor ligands and their potential for novel drugs,” Biochimica et Biophysica Acta 2011, 1808, 1290-1308);

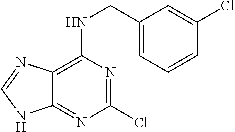 [SMILES: Clc1cccc(CNc2nc(Cl)nc3[nH]cnc23)c1]

### (MRS5930; see Jacobson K A et al. “John Daly Lecture: Structure-guided Drug Design for Adenosine and P2Y Receptors,” Comp. and Struct. Biotechnology Jour 13. 286-298. 2015);

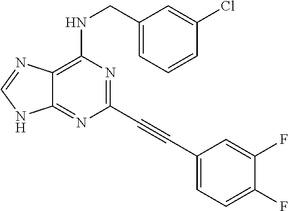 [SMILES: Fc1ccc(C#Cc2nc(NCc3cccc(Cl)c3)c3nc[nH]c3n2)cc1F]

(MRS5923; see Jacobson K A et al. “John Daly Lecture: Structure-guided Drug Design for Adenosine and P2Y Receptors,” Comp. and Struct. Biotechnology Jour 13. 286-298. 2015);

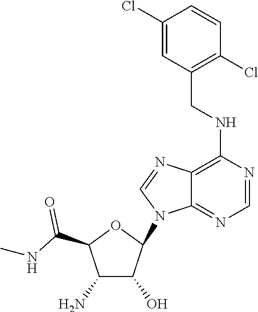 [SMILES: CNC(=O)[C@H]1O[C@@H](n2cnc3c(NCc4cc(Cl)ccc4Cl)ncnc32)[C@H](O)[C@@H]1N]

(see Tracey W R et al. “Novel n6-substitued adenosine 5′-N-methyluronamides with high selectivity for human A3R reduce ischemic myochardial injury,” Am J Physiol Heart Circ Physiol 285. 2003; Muller C E, Jacobson K A, “Recent Developments in adenosine receptor ligands and their potential for novel drugs,” Biochimica et Biophysica Acta 1808, 1290-1308. 2011; and Wan T C et al. “The A3R Agonist CP-532,903 Protects against Myocardial Ischemia/Reperfusion Injury,” J. of Pharmacology and Exptl Therapies 324,1. 2008);

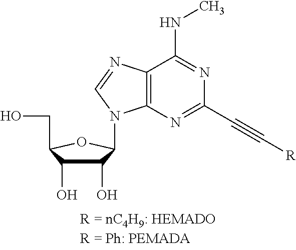

(see Volpini R et al. “HEMADO as Potent and Selective Agonists of hA3R,” J Med Chem 45, 3271-3279. 2002; Muller C E et al. “Recent Developments in adenosine receptor ligands and their potential for novel drugs,” Biochimica et Biophysica Acta 1808, 1290-1308. 2011; and Volpini R et al. “Synthesis and Evaluation of Potent and Highly Selective Agonists for hA3R,” J of Med Chem 52, 7897-7900. 2009);

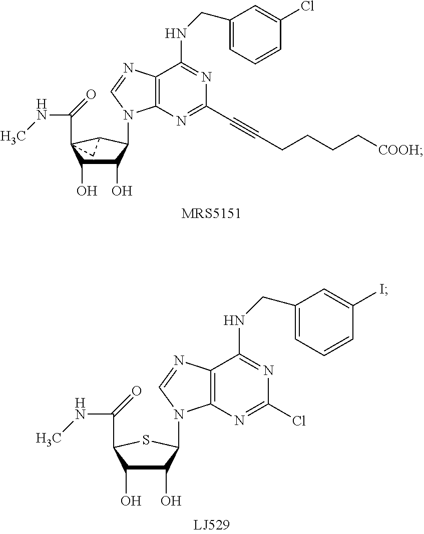

(see Muller C E, Jacobson K A. “Recent Developments in adenosine receptor ligands and their potential for novel drugs,” Biochimica et Biophysica Acta 1808, 1290-1308. 2011);

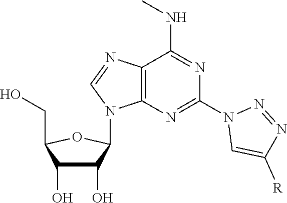

wherein R is H or cyclopentylmethyl;

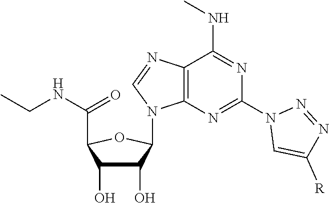

wherein R is H, butyl, or pyridin-2-yl (see Cosyn L. et al., “2-triazole-substituted adenosines,” J Med Chem 2006. 49. 7373-7383);

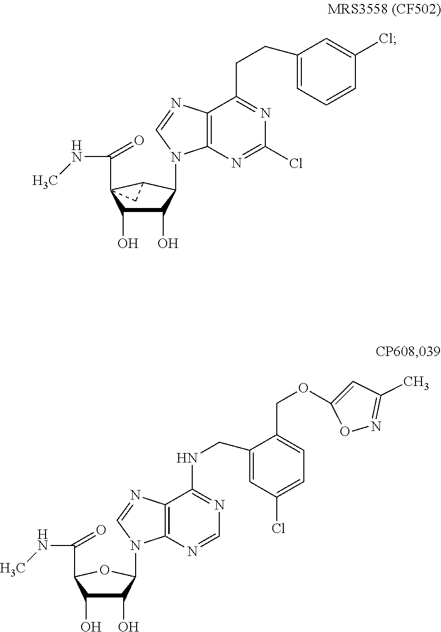

(see Muller C E, Jacobson K A. “Recent Developments in adenosine receptor ligands and their potential for novel drugs,” Biochimica et Biophysica Acta 1808, 1290-1308. 2011);

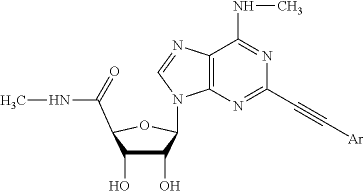

wherein Ar is selected from phenyl, p-CH₃CO-phenyl, p-fluorophenyl, or 2-pyridyl (see Volpini R et al. “Synthesis and Evaluation of Potent and Highly Selective Agonists for hA3R,” J Med Chem 52, 7897-7900. 2009);

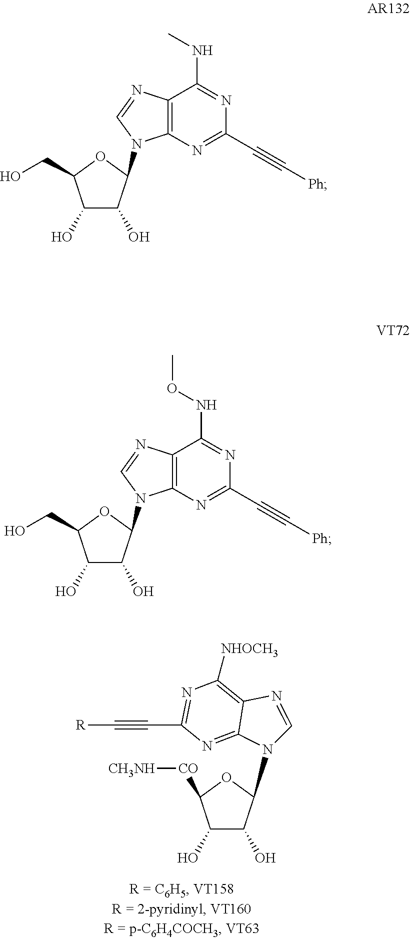

(see Pugliese A M et al., “Role of A3R on CA1 hippocampal neurotransmission during OGD,” Biochem Pharmacology 74. 2007);

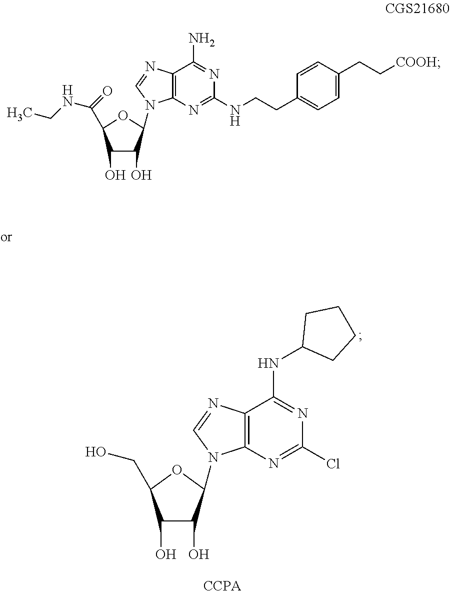

(see Klotz K N “Adenosine receptors and their ligands NS's,” Arch Pharmacol. 362. 382-391. 2000); or a pharmaceutically acceptable salt thereof. In some embodiments, the compound is selected from a (N)-methanocarba nucleoside such as those disclosed above; or a pharmaceutically acceptable salt thereof.

In some embodiments, the compound is selected from:

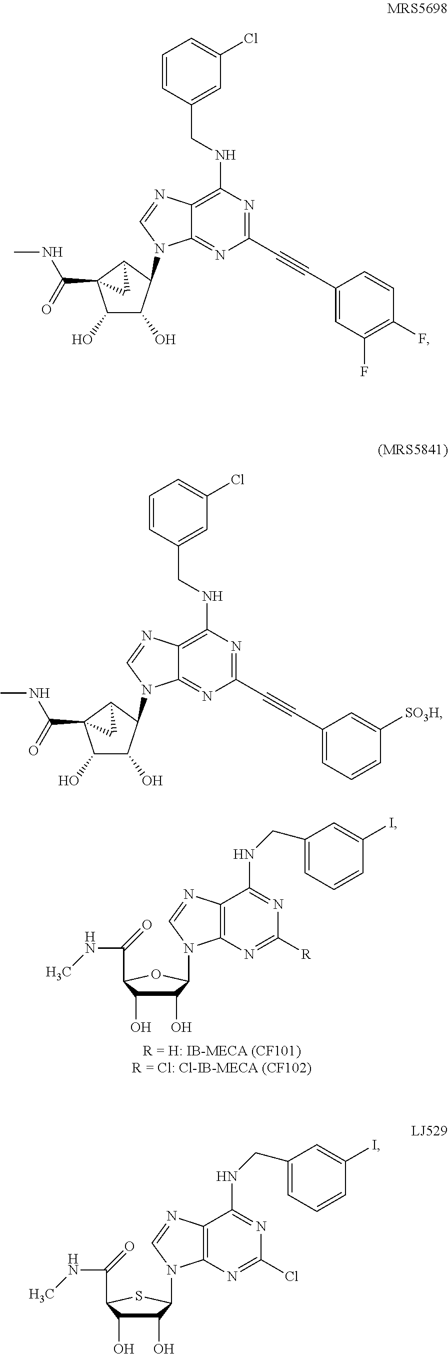

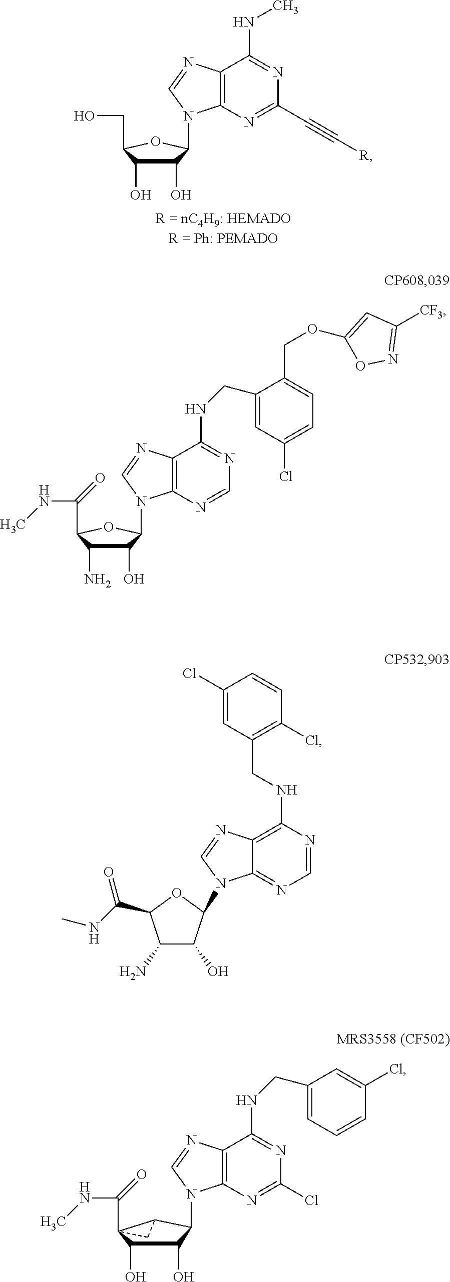

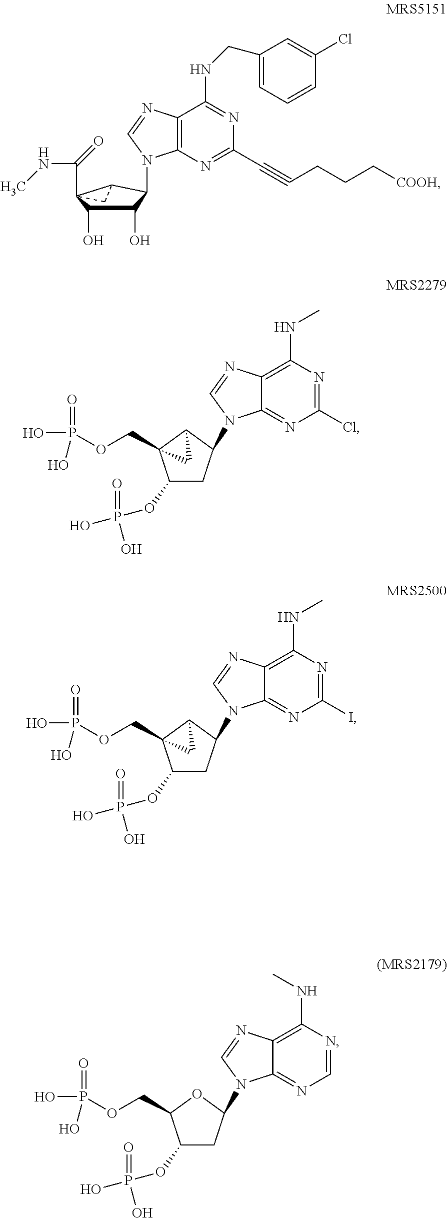

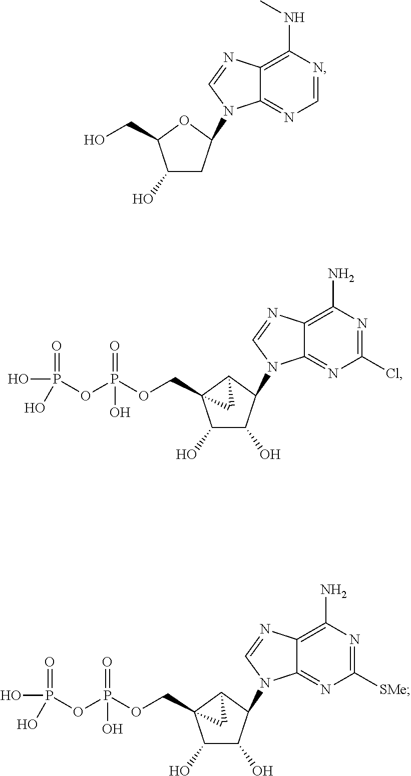 [SMILES: CNc1ncnc2c1ncn2[C@H]1C[C@H](O)[C@@H](CO)O1 ; Nc1nc(Cl)nc2c1ncn2[C@H]1[C@H](O)[C@H](O)[C@@]2(COP(=O)(O)OP(=O)(O)O)C[C@H]12 ; CSc1nc(N)c2ncn([C@H]3[C@H](O)[C@H](O)[C@@]4(COP(=O)(O)OP(=O)(O)O)C[C@H]34)c2n1]

or a pharmaceutically acceptable salt thereof. In some embodiments, the compound is selected from a (N)-methanocarba nucleoside such as those disclosed above; or a pharmaceutically acceptable salt thereof.

In some embodiments, the compound is

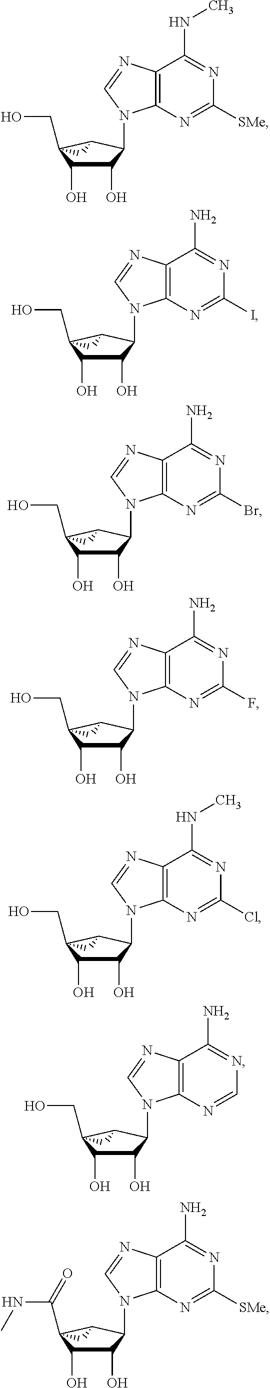

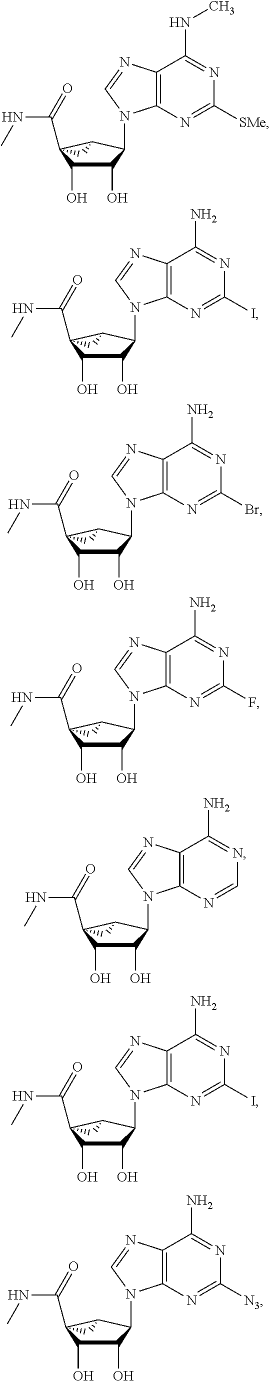

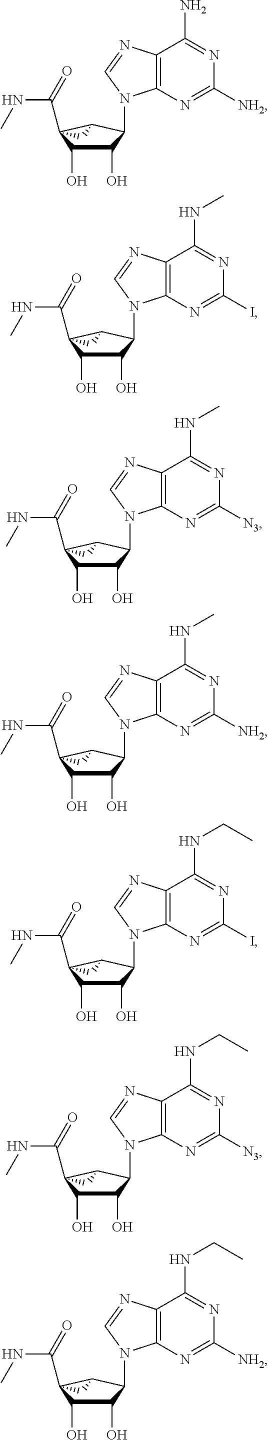

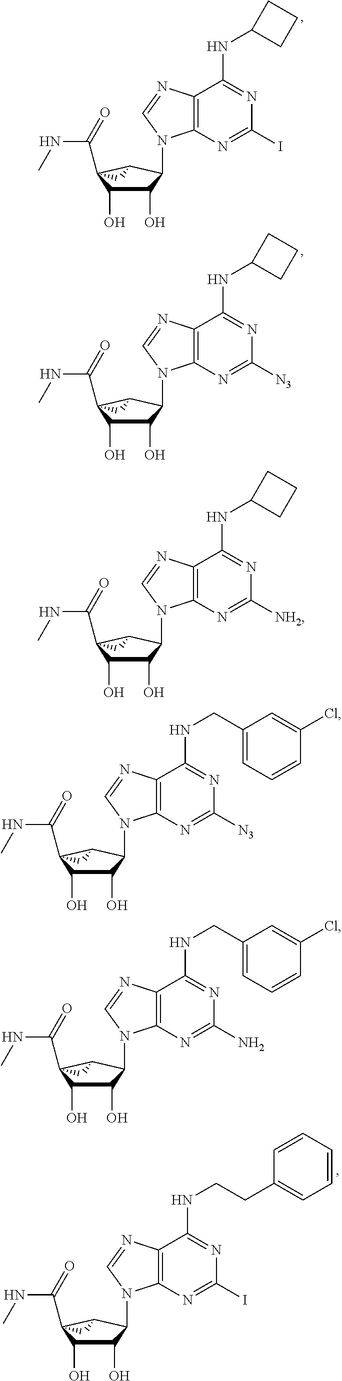

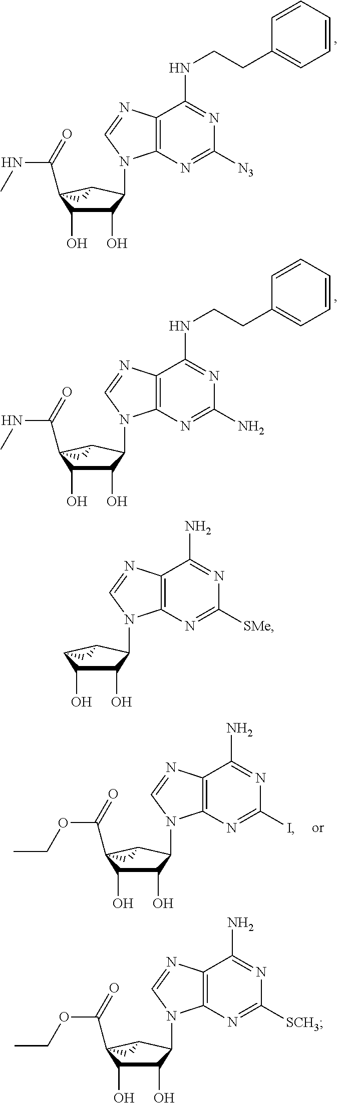

or a pharmaceutically acceptable salt thereof.

In some embodiments, the compound is

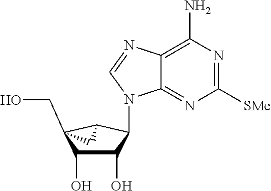

or a pharmaceutically acceptable salt thereof.

In some embodiments, the compound is

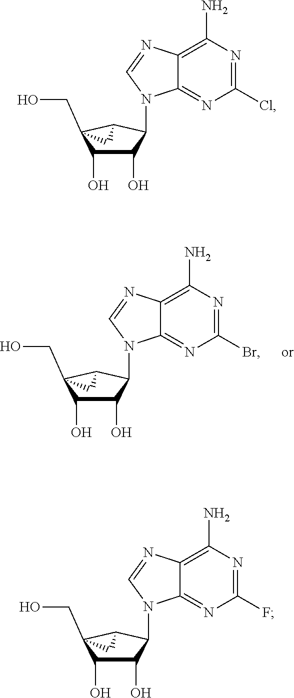

or a pharmaceutically acceptable salt thereof.

In some embodiments, the compound is

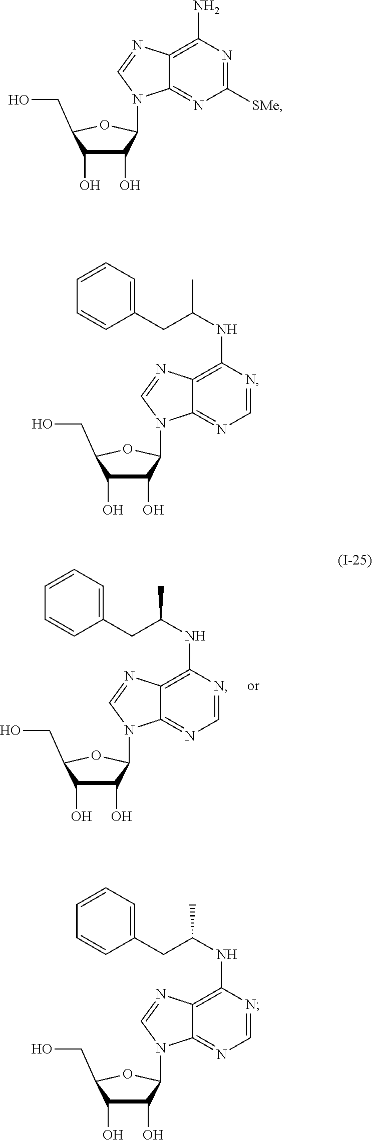

or a pharmaceutically acceptable salt thereof. In some embodiments, the compound is I-25 or a pharmaceutically acceptable salt thereof.

In some embodiments, the compound is MRS5698, MRS5980, or BIO-205:

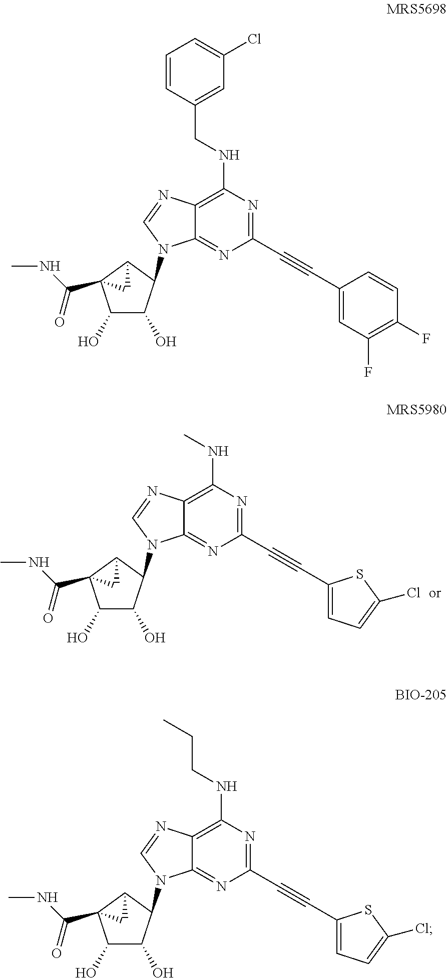 [SMILES: CNC(=O)[C@]12C[C@@H]1[C@@H](n1cnc3c(NCc4cccc(Cl)c4)nc(C#Cc4ccc(F)c(F)c4)nc31)[C@H](O)[C@@H]2O ; CNC(=O)[C@]12C[C@@H]1[C@@H](n1cnc3c(NC)nc(C#Cc4ccc(Cl)s4)nc31)[C@H](O)[C@@H]2O ; CCCNc1nc(C#Cc2ccc(Cl)s2)nc2c1ncn2[C@H]1[C@H](O)[C@H](O)[C@@]2(C(=O)NC)C[C@H]12]

or a pharmaceutically acceptable salt thereof. In some embodiments, the compound is MRS5698, MRS5980, or BIO-205, or a pharmaceutically acceptable salt thereof, and the injury, disease, or disorder is pain, a pain condition, or a pain disorder. In some embodiments, the pain, pain condition, or pain disorder is neuropathic pain.

In some embodiments, the compound is selected from one of those in Table 1, or a pharmaceutically acceptable salt thereof. In some embodiments, the compound is a mono-, di-, or tri-phosphate of a compound of Formula I, I-A, or I-B, such as a compound depicted in Table 1, or a pharmaceutically acceptable salt thereof, or a prodrug thereof. In some embodiments, the prodrug of the mono-, di-, or tri-phosphate is a corresponding mono-, di-, or tri-phosphate ester such as an alkyl or phenyl ester thereof. Exemplary prodrugs of phosphates are described in U.S. Pat. No. 9,724,360, the contents of which are hereby incorporated by reference.

TABLE 1
Exemplary Compounds of the Present Invention

| Compound # | Structure |
|---|---|
| I-1 (AST-004) | 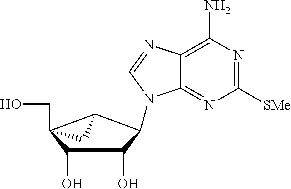 |
| I-2 | 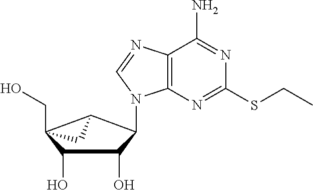 |
| I-3 | 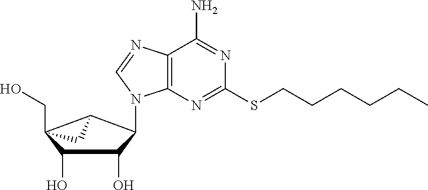 |
| I-4 | 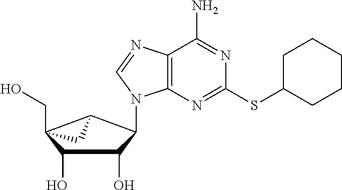 |
| I-5 | 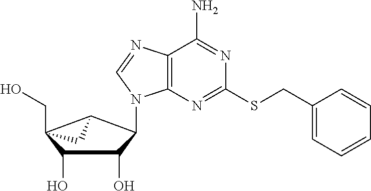 |
| I-6 | 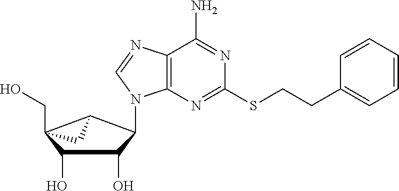 |
| I-7 | 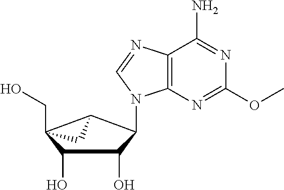 |
| I-8 | 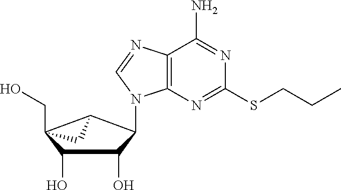 |
| I-9 | 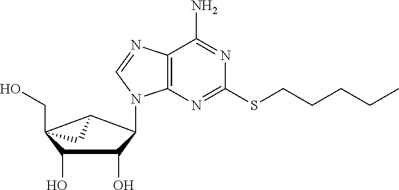 |
| I-10 | 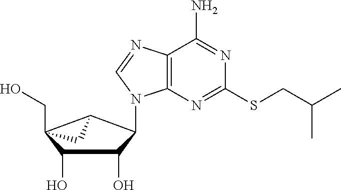 |
| I-11 | 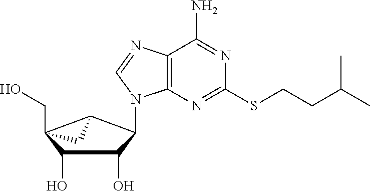 |
| I-12 | 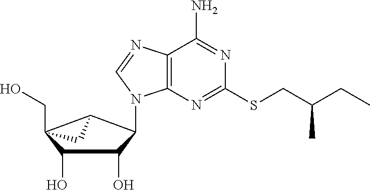 |
| I-13 | 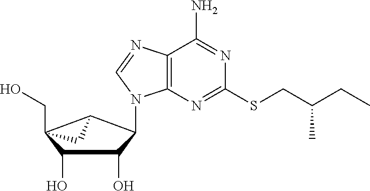 |
| I-14 | 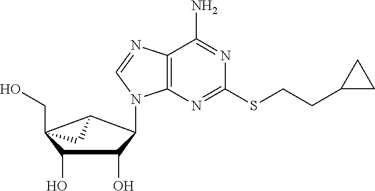 |
| I-15 | 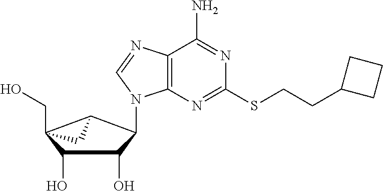 |
| I-16 | 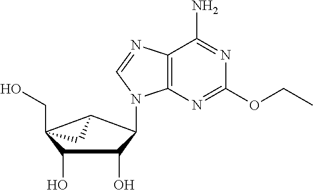 |
| I-17 | 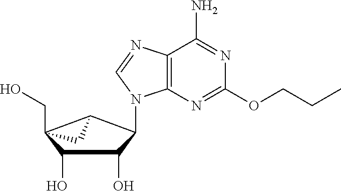 |
| I-18 | 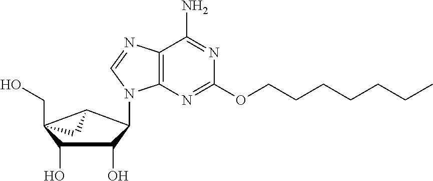 |
| I-19 | 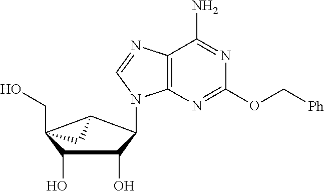 |
| I-20 | 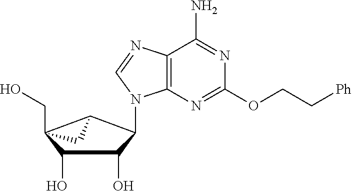 |
| I-21 | 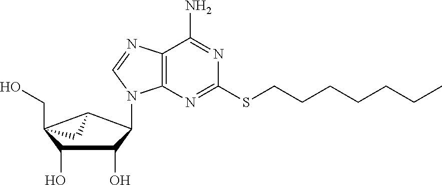 |
| I-22 | 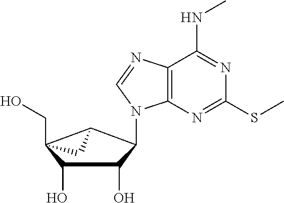 |
| I-23 (AST-008) | 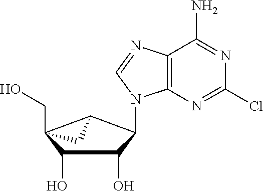 |
| I-24 | 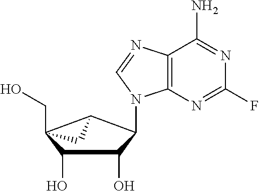 |
| I-25 R-(—)-PIA, N⁶-(L-2- Phenylisopropyl)adenosine | 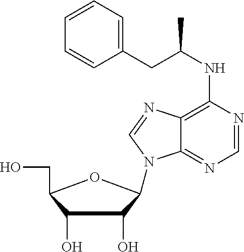 |
| I-26 | 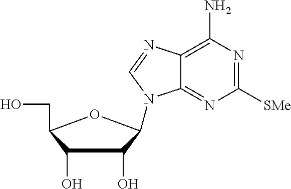 |

### Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry Thomas Sorrell, University Science Books, Sausalito: 1999, and March 's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

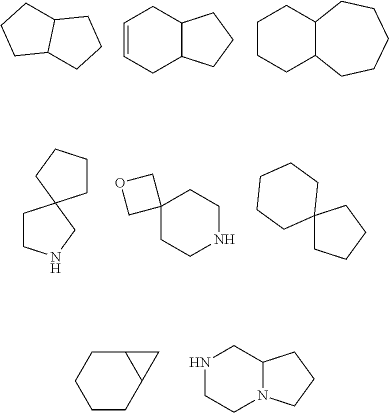

### Exemplary Bridged Bicyclics Include:

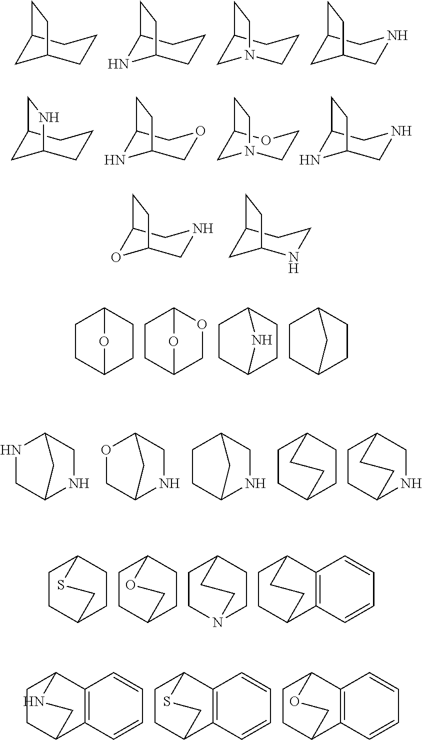

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR* (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A “substituted” alkylene chain is a polymethylene group in which one or more methylene hydrogen atoms are replaced with a substituent. Suitable substituents include those described below for a substituted aliphatic group.

The term “alkenylene” refers to a bivalent alkenyl group having at least one carbon-carbon double bond. Unless otherwise specified, the double bond may be cis or trans. In some embodiments, an alkenylene group has a single carbon-carbon double bond. In some embodiments, the double bond is cis. In some embodiments, the double bond is trans. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group having at least one carbon-carbon triple bond. A carbon-carbon triple bond may be located at an internal or terminal location in the alkynylene group, i.e., at either end or between two carbon atoms internal to the chain or carbon atoms. A substituted alkynylene chain is a polymethylene group containing at least one triple bond in which one or more hydrogen atoms are replaced with a substituent. Suitable substituents include those described below for a substituted aliphatic group. In some embodiments, the triple bond is at the terminal position and the alkynyl hydrogen is optionally replaced by a substituent.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar—,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or *NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₄Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R⁶⁰² is independently hydrogen, C₁₋₆ aliphatic, —CH₂Ph, —O(CH₂)₀₋₁Ph, —CH₂—(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{∘}₃, —OSiR^{∘}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*2))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R, —S(O)₂NR^{†}₂, —C(S)NR\₂, —C(NH)NR^{†}₂, or —N(RT)S(O)₂RT; wherein each RT is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when RT is C₁₋₆ aliphatic, RT is optionally substituted with halogen, —R*, -(haloR*), —OH, —OR*, —O(haloR*), —CN, —C(O)OH, —C(O)OR^{•}, —NH₂, —NHR*, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### Synthesis of Compounds

Compounds described herein, such as compounds of Formula I, I-A, or I-B and pharmaceutically acceptable salts thereof, may be synthesized using methods known in the art, such as methods described in U.S. Pat. No. 9,789,131, which is hereby incorporated by reference in its entirety. For example, such compounds may generally be prepared according to Scheme I set forth below:

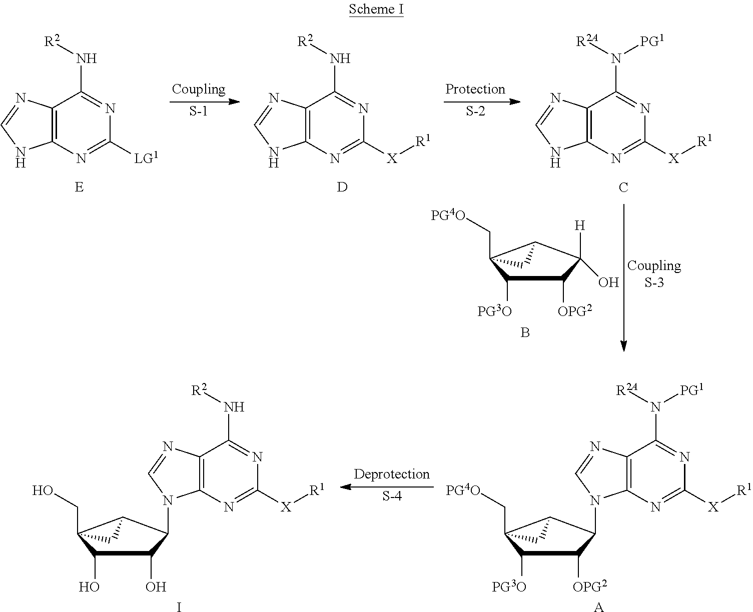

In Scheme I above, each of X, R¹, R², R^{2A}, PG¹, PG², PG³, and PG⁴ is as defined and described in embodiments herein, both singly and in combination.

General principles of organic chemistry and synthesis, well known in the art, are described in, for example, Organic Chemistry, Thomas Sorrell, University Science Books, Sausalito: 1999; March's Advanced Organic Chemistry, 5^{th} Ed., Ed.: Smith, M. B. and March, J., John Wiley & Sons, New York: 2001; and Comprehensive Organic Synthesis, 2^{nd} Ed., Ed.: Knochel, P. and Molander, G. A., Elsevier, Amsterdam: 2014; the entire contents of each of which are hereby incorporated by reference.

At step S-1, a thiol or alcohol of formula R¹—X—H is coupled with an adenine nucleobase of formula E. The coupling is conducted in the presence of a suitable base. Alternatively, the corresponding thiol or alcohol metal salt of formula R¹—X-M (wherein M is a metal atom, such as sodium or potassium), is coupled with an adenine nucleobase of formula E.

The LG¹ group of formula E is a suitable leaving group. Suitable leaving groups are well known in the art, as described in, for example, the references described above. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. Examples of suitable leaving groups include chloro, iodo, bromo, fluoro, methanesulfonyl (mesyl), tosyl, triflate, nitro-phenylsulfonyl (nosyl), and bromo-phenylsulfonyl (brosyl). For example, LG¹ may be chloro, fluoro, or triflate. In certain embodiments, LG¹ is chloro. When —X—R¹ is halogen in Formula I or I-B above, step S-1 is omitted (LG¹ is halogen, e.g., chloro, and does not need to undergo any chemical transformation).

At step S-2, adenine 2-halo, 2-thioether, or 2-ether nucleobase D is protected to afford N-protected adenine 2-halo, 2-thioether or 2-ether nucleobases of formula C.

The PG¹ group of formulae C and A is a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the —N(R^{2A})— moiety to which it is attached, include, but are not limited to, aralkylamines, carbamates, allyl amines, amides, and the like. Examples of PG¹ groups of formulae C and A include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, and the like. In some cases, PG¹ is an acid-labile amino protecting group. PG¹ taken with the —N(R^{2A})— moiety to which it is attached can be an acid-labile carbamate. Typically, PG¹ is BOC.

One of ordinary skill in the art will recognize that when R² in nucleobase D is hydrogen, R^{2A} in nucleobase C may be hydrogen (from addition of a single protecting group to nucleobase D) or a suitable amino protecting group (from addition of a second protecting group to nucleobase D), depending on the reaction conditions (for example, the stoichiometry of nucleobase D relative to protecting group reagents). R^{2A} is hydrogen or can be a suitable amino protecting group, e.g., BOC. In some cases, PG¹ and R^{2A} are each BOC.

At step S-3, an N-protected adenine 2-halo, 2-thioether or 2-ether nucleobase of formula C undergoes coupling with protected (N)-methanocarba sugar analogue B to afford (N)-methanocarba nucleoside analogue A. The coupling can be conducted under Mitsunobu-type conditions.

Each of the PG², PG³, and PG⁴ groups of formulae B and A is independently a suitable hydroxyl protecting group. Suitable hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Each of PG², PG³, and PG⁴, taken with the oxygen atom to which it is bound, may be selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, or carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl) ethoxymethyl, and tetrahydropyranyl ethers. Examples of arylalkyl ethers include benzyl, p-methoxybenzyl (MPM), 3,4-dimethoxybenzyl, o-nitrobenzyl, p-nitrobenzyl, p-halobenzyl, 2,6-dichlorobenzyl, p-cyanobenzyl, trityl, 2- and 4-picolyl.

For example, each of PG², PG³, and PG⁴ can be an acid-labile hydroxyl protecting group.

In some cases, PG⁴ taken with the oxygen atom to which it is bound is a silyl ether or arylalkyl ether. Alternatively, PG⁴ is trityl or dimethoxy trityl.

PG² and PG³ taken together with the oxygen atoms to which they are bound can form a diol protecting group, such as a cyclic acetal or ketal. Such groups include methylene, ethylidene, benzylidene, isopropylidene, cyclohexylidene, and cyclopentylidene, a silylene derivative such as di-t-butylsilylene and a 1,1,3,3-tetraisopropyldisiloxanylidene derivative, a cyclic carbonate, and a cyclic boronate.

At step S-4, the (N)-methanocarba nucleoside analogue A is deprotected to provide a compound of Formula I, I-A, or I-B. One of ordinary skill in the art will recognize that the conditions required to deprotect each of PG¹, PG², PG³, and PG⁴ may be the same or different. When more than one set of conditions is required to remove all four of PG¹, PG², PG³, and PG⁴ the deprotection steps may be carried out with, or without, isolation of intermediates where one or more, but not all, of PG¹, PG², PG³, and PG⁴ have been deprotected.

### Exemplary Methods of Treatment

In some embodiments, the present invention provides a method of treating an injury, disease, or condition selected from traumatic brain injury (TBI), concussion, stroke (e.g., acute ischemic stroke (AIS)), partial or total spinal cord transection, malnutrition, toxic neuropathies, meningoencephalopathies, neurodegeneration caused by a genetic disorder, age-related neurodegeneration, vascular disease, Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), cardiovascular disease, autoimmune diseases, allergic diseases, transplant rejection, graft-versus-host disease, intraocular hypertension, glaucoma, odor sensitivity, an olfactory disorder, type 2 diabetes, pain control, respiratory diseases, deficits in CNS function, deficits in learning, deficits in cognition, otic disorders, Meniere's disease, endolymphatic hydrops, progressive hearing loss, dizziness, vertigo, tinnitus, collateral brain damage associated with radiation cancer therapy, migraine treatment, sleep disorders in the elderly, epilepsy, schizophrenia, symptoms experienced by recovering alcoholics, damage to neurons or nerves of the peripheral nervous system during surgery, gastrointestinal conditions, pain mediated by the CNS, migraine, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties, vertigo, dysarthria, dysphagia, ocular abnormalities or disorientation, or addiction; comprising administering to a patient a compound described herein, or a pharmaceutically acceptable salt thereof or a composition comprising the same. In some embodiments, the present invention provides a method of treating an injury, disease, or condition selected from traumatic brain injury (TBI), stroke, a neurodegenerative condition, or a heart or cardiovascular disease, comprising administering to a patient in need thereof an effective amount of a compound described herein, or a pharmaceutically acceptable salt thereof or composition comprising the same.

In some embodiments, the compound acts as an agonist of an A₃ adenosine receptor (A₃R). In some embodiments, the compound is a partial A₃R agonist. In some embodiments, the compound is a biased A₃R agonist. In some embodiments, the compound acts by dual agonism at an A₃ adenosine receptor and an A₁ adenosine receptor (A₁R). In some embodiments, the compound acts as an agonist of an A₁ adenosine receptor (A₁R). In some embodiments, the compound is a partial A₁R agonist. In some embodiments, the compound is a biased A₁R agonist.

In some embodiments, the present invention provides a method of treating an injury, disease, or condition selected from traumatic brain injury (TBI), stroke, a neurodegenerative condition, or a heart or cardiovascular disease, comprising administering to a patient in need thereof an effective amount of compound described herein, or a pharmaceutically acceptable salt thereof or composition comprising the same.

In some embodiments, the present invention provides a method of treating a brain or central nervous system (CNS) injury or condition selected from traumatic brain injury (TBI) or stroke, comprising administering to a patient in need thereof an effective amount of a compound described herein, or a pharmaceutically acceptable salt thereof or composition comprising the same.

In some embodiments, the present invention provides a method of treating or ameliorating a traumatic brain injury (TBI), radiation damage, stroke, migraine headache, a heart or cardiovascular disease, or neurodegenerative disorder, comprising administering to a patient in need thereof an effective amount of a compound described herein, or a pharmaceutically acceptable salt thereof or composition comprising the same.

In some embodiments, the present invention provides a method of treating or ameliorating a traumatic brain injury (TBI), radiation damage, stroke, migraine headache, a heart or cardiovascular disease, or neurodegenerative disorder, comprising administering to a patient in need thereof an effective amount of a compound described herein, or a pharmaceutically acceptable salt thereof or composition comprising the same.

In some embodiments, the present invention provides a method of treating an injury, disease, or condition selected from traumatic brain injury (TBI), stroke, a neurodegenerative condition, or a heart or cardiovascular disease comprising administering to a patient in need thereof an effective amount of a compound described herein, or a pharmaceutically acceptable salt thereof or composition comprising the same.

In some embodiments, the injury, disease, or condition is TBI.

In some embodiments, the TBI is selected from concussion, blast injury, combat-related injury, or a mild, moderate or severe blow to the head.

In some embodiments, the injury, disease, or condition is a stroke selected from ischemic stroke, hemorrhagic stroke, subarachnoid hemorrhage, cerebral vasospasm, or transient ischemic attacks (TIA).

In some embodiments, neuroprotection or neurorestoration is increased in the patient as compared with an untreated patient.

In some embodiments, the neurodegenerative disease is selected from Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), or a neurodegenerative condition caused by a virus, alcoholism, tumor, toxin, or repetitive brain injuries.

In some embodiments, the neurodegenerative disease is Parkinson's Disease.

In some embodiments, the injury, disease, or condition is Alzheimer's Disease, migraine, brain surgery, or a neurological side effect associated with cancer chemotherapy.

In some embodiments, the recovery period after the TBI, stroke, cardiac ischemia, or myocardial infarction is decreased as compared with an untreated patient.

In some embodiments, the heart or cardiovascular disease is selected from cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, or atherosclerosis.

In some embodiments, the heart or cardiovascular disease is cardiac ischemia or myocardial infarction.

In some embodiments, the compound or composition is administered chronically to treat stroke, cardiac ischemia, or myocardial infarction during the time period after the injury has occurred as it resolves.

In some embodiments, the present invention provides a method of increasing neuroprotection or neurorestoration in a patient in need thereof who has suffered a TBI or stroke, comprising administering to the patient an effective amount of a compound described herein, or a pharmaceutically acceptable salt thereof or composition comprising the same.

In some embodiments, the compound or pharmaceutically acceptable salt thereof is administered orally, intravenously, or parenterally.

In some embodiments, the compound or composition is administered within 24 hours of the TBI or stroke.

In some embodiments, the compound or composition is administered within 8 hours of the TBI or stroke.

In some embodiments, the compound or composition is administered at least during the first 8-48 hours following the TBI or stroke.

In some embodiments, the present invention provides a method of treating a heart or cardiovascular disease comprising administering to a patient in need thereof an effective amount of a compound described herein, or a pharmaceutically acceptable salt thereof or composition comprising the same.

In some embodiments, the patient has suffered a cardiac ischemia or myocardial infarction.

In some embodiments, the compound or composition increases cardioprotection or regeneration of damaged heart tissue in the patient.

In some embodiments, the compound or composition decreases the recovery period after the cardiac ischemia or myocardial infarction in the patient as compared with an untreated patient.

In some embodiments, the present invention provides a method of treating an injury, disease, disorder, or condition selected from:
(i) brain damage caused by radiation or collateral brain damage associated with radiation cancer therapy or migraine treatment;
(ii) migraine headache;
(iii) a condition associated with a brain injury or a neurodegenerative condition; or
(iv) an autoimmune disease or condition, glaucoma, an otic disorder, progressive hearing loss, tinnitus, epilepsy, or pain (e.g., pain mediated by the CNS, neuropathic pain, inflammatory pain, or acute pain);
   comprising administering to a patient in need thereof an effective amount of a compound described herein, or a pharmaceutically acceptable salt thereof or composition comprising the same.

In some embodiments, the compound or composition increases neuroprotection or neurorestoration in the patient as compared with an untreated patient.

In some embodiments, the condition associated with a brain injury or a neurodegenerative condition is selected from epilepsy, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties or deficit in cognition, deficit in CNS function, deficit in learning, vertigo, dysarthria, dysphagia, ocular abnormalities, or disorientation.

In some embodiments, the present invention provides a method of increasing cardioprotection or regeneration of damaged heart tissue in a patient in need thereof who has suffered a cardiac ischemia or myocardial infarction, comprising administering to the patient an effective amount of a compound described herein, or a pharmaceutically acceptable salt thereof or composition comprising the same.

In some embodiments, the present invention provides a method of treating a disease, disorder, or condition selected from deficit in cognition, deficit in CNS function, deficit in learning, and memory loss, comprising administering to a subject in need thereof an effective amount of a compound described herein, or a pharmaceutically acceptable salt thereof or a pharmaceutically acceptable composition thereof.

In some embodiments, the disease, disorder, or condition is deficit in cognition.

In some embodiments, the disease, disorder, or condition is deficit in CNS function.

In some embodiments, the disease, disorder, or condition is deficit in learning.

In some embodiments, the disease, disorder, or condition is memory loss.

In some embodiments, the subject has suffered one or more traumatic brain injuries (TBI or TBIs) and the disease, disorder, or condition is associated with the TBI or TBIs.

In some embodiments, the subject has suffered one or more strokes and the disease, disorder, or condition is associated with the one or more strokes.

In some embodiments, the subject has suffered one or more ischemic strokes, hemorrhagic strokes, subarachnoid hemorrhages, cerebral vasospasms, or transient ischemic attacks (TIA).

In some embodiments, the subject has Alzheimer's disease and the disease, disorder, or condition is associated with the Alzheimer's disease.

In some embodiments, a method provided herein improves cognitive or neurological function as measured by a score increase between about 1% and 40% in the delayed verbal recall task of the revised Wechsler Memory Scale.

In some embodiments, a method provided herein improves the score between about 5-10%, 10-20%, 15-30%, 20-30%, 30-40%, or 5-30% in the delayed verbal recall task of the revised Wechsler Memory Scale.

In some embodiments, the method increases synaptic plasticity, improves hippocampal long-term potentiation, improves cognitive function, decreases cognitive impairment, and/or improves or restores memory or learning.

In some embodiments, the method increases synaptic plasticity, improves hippocampal long-term potentiation, improves cognitive function, decreases cognitive impairment, prevents or delays cognitive decline, decreases plaque burden, enhances beta amyloid clearance, and/or improves or restores memory or learning.

In some embodiments, the method improves or enhances cognition or neurological function by enhancing synaptogenesis.

In one aspect, the present invention provides a method of improving cognitive or neurological function in a subject having Alzheimer's disease, comprising administering to a subject in need thereof an effective amount of a disclosed compound such as I-1, or a pharmaceutically acceptable salt thereof or a pharmaceutically acceptable composition thereof. In some embodiments, the improvement in cognitive or neurological function as measured by a score increase between about 1% and 40%, or about 5-10%, 10-20%, 15-30%, 20-30%, 30-40%, or 5-30%, in the delayed verbal recall task of the revised Wechsler Memory Scale.

It has been surprisingly found that certain purine nucleoside mono-, di-, and tri-phosphates, such as phosphates of nucleosides disclosed herein, are dephosphorylated in vivo and exist primarily as the nucleoside, i.e., they are not substantially phosphorylated in vivo. Without wishing to be bound by theory, it is believed that such dephosphorylation is effected by ectonucleotidases, enzymes responsible for the dephosphorylation of nucleotides that are present both on the surface of cell membranes and circulating in blood and plasma (See Ziganshin et al. Pflugers Arch. (1995) 429:412-418). It is often extremely difficult to predict which nucleotide analogs will be substrates for ectonucleotidases and will thus be expected to be dephosphorylated in vivo. In some embodiments, the dephosphorylated compound is responsible for the therapeutic efficacy. Thus, in some embodiments the corresponding, phosphorylated mono-, di-, or tri-phosphate, or a phosphate ester such as an alkyl or phenyl ester thereof, is a prodrug or precursor to the agent responsible for the therapeutic effect.

In some embodiments, compounds of the present invention are able to cross the blood-brain barrier (BBB). The term “blood-brain barrier” or “BBB,” as used herein, refers to the BBB proper as well as to the blood-spinal barrier. The blood-brain barrier, which consists of the endothelium of the brain vessels, the basal membrane and neuroglial cells, acts to limit penetration of substances into the brain and cerebrospinal fluid (CSF). In some embodiments, the brain/plasma ratio of total drug is at least approximately 0.01 after administration (e.g. oral or intravenous administration) to a patient. In some embodiments, the brain/plasma ratio of total drug is at least approximately 0.03. In some embodiments, the brain/plasma ratio of total drug is at least approximately 0.06. In some embodiments, the brain/plasma ratio of total drug is at least approximately 0.1. In some embodiments, the brain/plasma ratio of total drug is at least approximately 0.2.

Prototypical adenosine A₃ agonists such as Cl-IB-IMECA and MRS5698 are low-solubility, lipophilic compounds with cLogP values typically >2. This lipophilicity is a major factor contributing to these compounds' high plasma protein binding, high brain binding and resulting low free fraction of drug available to interact with the A₁ and/or A₃ receptor in the brain. In some embodiments, for example neurological and neurodegenerative conditions, the physicochemical properties of compounds of the present invention are substantially different; these and related compounds are hydrophilic compounds with cLogP <0, resulting in high solubility, low plasma and brain binding and high unbound drug concentrations available to interact with the A₁ and/or A₃ receptor.

Accordingly, in some embodiments the compound has a cLogP less than about 0.8, about 0.7, about 0.6, about 0.5, about 0.4, about 0.3, about 0.2, about 0.1, about 0.05, about 0.01, or about 0.005. In some embodiments, the compound has a cLogP less than 0, such as less than about −0.1, −0.2, −0.3, −0.4, −0.5, −0.6, −0.7, −0.8, or −0.9 or less. In some embodiments, the compound has an unbound fraction in plasma of about 0.5 to 0.9. In some embodiments, the compound has an unbound fraction in plasma of about 0.6 to 0.85, 0.7 to 0.8, or about 0.75. In some embodiments, the compound has an unbound fraction in brain of at least about 0.02, or at least about 0.03, 0.04, 0.05, 0.06, 0.07, 0.08, 0.09, 0.1, 0.12, 0.15, or 0.17 or greater. In some embodiments, the compound has an unbound fraction in plasma of about 0.6 to 0.85, 0.7 to 0.8, or about 0.75 and/or at least 0.08 unbound fraction in brain.

### Uses of Compounds and Pharmaceutically Acceptable Compositions Thereof

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

### Brain, CNS, Cardiovascular, and Other Injuries and Conditions

In some embodiments, the present invention provides a new approach to preventing and/or treating brain damage associated with acute brain trauma as well as longer term diseases of the brain and CNS and heart and cardiovascular diseases and conditions. In one aspect, the present invention provides methods of treating such injuries, diseases, and conditions by utilizing neuroprotective and neurorestorative effects mediated by astrocytes, which are now understood as the key natural caretaker cell of neurons, as well as the astrocyte mitochondria, which supply a significant portion of the brain's energy. In another aspect, the present invention provides methods of treating such injuries, diseases, and conditions by cardioprotective and regenerative effects mediated by A₃R receptors. Regarding neuroprotective and neurorestorative effects, without wishing to be bound by theory, it is believed that selective enhancement of astrocyte energy metabolism mediated by A₃R and/or P2Y₁ receptors promotes astrocyte caretaker functions, such as their neuroprotective and neurorestorative functions, in turn enhancing the resistance of neurons and other cells to both acute injury and long-term stress. In some cases, it may be advantageous to achieve biased, i.e., selective or preferential, of one or more pathways mediated by A₃R and/or P2Y₁ and/or A₁R receptors wherein one or more undesired pathways are not activated or activated to a lesser degree. In addition to or as an alternative to astrocytes, neuroprotective or neurorestorative function of glia, microglia, neurons, endothelium cells and other brain and/or CNS cell types may be activated. Accordingly, in one aspect, the present invention provides compounds and methods of use thereof for treating, ameliorating, or promoting recovery from certain conditions of the brain or central nervous system (CNS) such as brain injuries, for example by increasing neuroprotection and/or neurorestorative effects mediated by astrocytes, glia, microglia, neurons, endothelium cells or other cells of the brain and/or CNS, comprising administering to a patient in need thereof an effective amount of a compound described herein, or a pharmaceutically acceptable salt thereof or composition comprising the same.

Astrocytes play key roles in supporting and protecting neurons and they critically affect the outcome of brain injuries that cause brain damage, such as ischemic injuries. The central role astrocyte mitochondria themselves play in these brain functions is less well appreciated. For example, inhibition of astrocyte mitochondria increases swelling and leads to necrotic cell death. Neurons are permanently injured by recurrent spreading depolarizations only if astrocyte mitochondrial function fails, and astrocyte mitochondria are required for reduction of pathophysiological elevations of extracellular K⁺, which initiate spreading depolarizations. Activation of purinergic receptors on astrocytes results in increased mitochondrial Ca²⁺ that enhances mitochondrial citric acid cycle function and increases respiration and ATP production. Accordingly, in one aspect, the present invention relates to the discovery that activation of astrocyte purinergic receptors enhances brain cell survival signaling pathways, enabling both astrocyte and neuronal viability during oxidative stress. Furthermore, activated astrocytes generate and supply reduced glutathione, a key antioxidant that aids in the resistance of both astrocytes and neurons to oxidative stress. Thus, in one aspect, the present invention provides a method of modulating astrocyte purinergic receptors to promote survival and viability of one or more cell types in the brain of a patient after oxidative stress, such as oxidative stress caused by a brain injury, ischemia-reperfusion or a neurodegenerative condition, comprising administering to a patient in need thereof a compound described herein, or a pharmaceutically acceptable salt thereof or composition comprising the same.

In some embodiments, activation of astrocytes is achieved through contacting with a disclosed compound one or more purinergic receptors such as adenosine receptors (ARs), for example those associated with or expressed by astrocytes, thus modulating the activity of the one or more receptors. In some embodiments, through effects on adenosine receptors such as A₁, A_{2A}, A_{2B} and A₃ on astrocytes, the compound activates astrocytes to treat one or more disclosed diseases or conditions. In some embodiments, after administration to a patient in need thereof, a disclosed compound influences one or more astrocyte functions. In some embodiments, the astrocyte function is selected from glutamate uptake, reactive gliosis, swelling, or release of neurotrophic and neurotoxic factors that act to ameliorate metabolic stress and its consequences. In some embodiments, the compound is an AR agonist. In some embodiments, the purinergic receptor is an A₃ adenosine receptor (A₃R). In some embodiments, the compound is an A₃R agonist. In some embodiments, the compound is a partial agonist or biased agonist or biased partial agonist, at an A₃ receptor (A₃R), such as a human A₃ receptor (hA₃R). In some embodiments, the compound acts as an agonist of an A₁ adenosine receptor (A₁R). In some embodiments, the compound is a biased agonist at an A₁ and/or A₃ receptor. In some embodiments, the compound acts by dual agonism at an A₃R and an A₁R.

In another aspect, the present invention provides a method of treating or ameliorating a brain injury, disease, or condition, such as a brain injury resulting from a TBI or progressive neurodegenerative disorder, in a patient in need thereof, comprising administering to the patient an effective amount of a compound described herein, or a pharmaceutically acceptable salt thereof or composition comprising the same. In some embodiments, the subject has suffered a TBI, concussion, stroke, partial or total spinal cord transection, or malnutrition. In other embodiments, the subject has suffered toxic neuropathies, meningoencephalopathies, neurodegeneration caused by a genetic disorder, age-related neurodegeneration, or a vascular disease; or another disease disclosed in U.S. Pat. No. 8,691,775, which is hereby incorporated by reference. In some embodiments, the present invention provides a method of treating or ameliorating a brain injury, disease, or condition, such as a brain injury resulting from a TBI or progressive neurodegenerative disorder, in a patient in need thereof, comprising administering to the patient an effective amount of a compound described herein, or a pharmaceutically acceptable salt thereof or composition comprising the same, wherein the compound is an A₁R and/or A₃R agonist. In some embodiments, the compound is a biased agonist, partial agonist, or biased partial agonist at an A₁ receptor. In some embodiments, the compound is a biased agonist, partial agonist, or biased partial agonist at an A₃ receptor. In some embodiments, the compound acts by dual agonism at an A₃R and an A₁R. In some embodiments, the compound is one of those depicted in Table 1, or a pharmaceutically acceptable salt thereof.

In another aspect, the present invention provides a method of promoting or increasing neuroprotection, neurorestoration, or neuroregeneration in a patient suffering from a disease or condition, comprising administering to the patient an effective amount of a compound described herein, or a pharmaceutically acceptable salt thereof or composition comprising the same. In some embodiments, the patient is suffering from a neurodegenerative disease or condition. In some embodiments, the patient has suffered a TBI or stroke.

### Traumatic Brain Injuries

Traumatic brain injuries (TBI) are a distressingly common medical condition and are predicted to become the third major cause of global morbidity and mortality by 2020. There are no approved treatments for TBI, and most TBI patients are discharged from the hospital with no pharmacological treatment (Witt 2006). Repetitive TBI such as concussions can trigger age-associated neurodegeneration that results in a range of symptoms and disabilities over decades (McKee 2013). TBIs can happen through sports-related injuries, motor vehicle accidents, falls, explosive impacts, physical assaults, etc. Injuries range widely in their complexity and severity, from “mild” concussions with brief alterations in mental status, cognitive difficulties, or loss of consciousness to “severe” with prolonged periods of unconsciousness and/or amnesia after the injury. In the U.S., approximately 1.7 million people have an injury resulting in a TBI annually and seek medical intervention (USCSF and CDC), and the CDC estimates that 1.6 to 3.8 million additional concussion incidents occur in sports and other recreational pursuits annually that do not present to hospital or emergency departments. (CDC; Langlois 2006) Approximately 5-10% of athletes will receive a concussion each sport season. (Sports Concussion Institute 2012) Football is the sport with the highest concussion risk for males (75% chance for concussion), while soccer has the highest concussion risk for females (50% chance for concussion). TBI is the leading cause of death and disability in children and young adults (CDC) and the most commonly received military-related injury; approximately 20% of U.S. Service Members deployed since 2003 have sustained at least one TBI. (Chronic Effects of Neurotrauma Consortium (CENC); Warden 2006; Scholten 2012; Taylor 2012; Gavett 2011; Guskiewicz 2005; Omalu 2005) Total TBI-related indirect and direct medical costs are estimated at $77 billion annually (UCSF and CDC). At least 5 million Americans require ongoing daily support in performing activities as a result of TBI (CDC and Thurman 1999).

Provided herein in one aspect is a method of treating TBI or promoting recovery from TBI, comprising administering to a patient in need thereof an effective amount of a compound described herein, or a pharmaceutically acceptable salt thereof or composition comprising the same. In some embodiments, the TBI is selected from traumatic injuries to the brain (such as concussion, blast injury, combat-related injury) or spinal cord (such as partial or total spinal cord transection). In some embodiments, the TBI results from a mild, moderate, or severe blow to the head, comprises an open or closed head wound, or results from a penetrating or non-penetrating blow to the head. In some embodiments, the present invention provides a method of treating TBI or promoting recovery from TBI, comprising administering to a patient in need thereof an effective amount of a compound described herein, or a pharmaceutically acceptable salt thereof or composition comprising the same, wherein the compound is an A₃R agonist. In some embodiments, the present invention provides a method of treating TBI or promoting recovery from TBI, comprising administering to a patient in need thereof an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is one of those described in Table 1, or a pharmaceutically acceptable composition comprising the same.

### Stroke

A stroke occurs when a blood vessel that transports oxygen and nutrients to the brain is disrupted due to an ischemic blockage or from the hemorrhagic rupture of a blood vessel in the brain, causing neurons, glia and endothelial cells in the disrupted region of the brain to die. The outcome of the stroke depends upon the location and breadth of damage, and the impacts of that damage are observed in the body functions regulated by the damaged brain region. Strokes can cause unilateral or bilateral paralysis, speech and language disabilities, memory loss, behavioral changes, and even death. Stroke is the fourth leading cause of death in the United States and is a major cause of adult disability. Each year, -800,000 people experience a new or recurrent stroke. Each day, over 2,000 Americans will have a stroke, resulting in death in over 400 of these incidents. Stroke accounted for ˜1 of every 19 deaths in the United States in 2010. An estimated 6.8 million Americans ≥20 years of age have had a stroke. (AHA and Go 2014) As of 2010, the annual direct and indirect cost of stroke was estimated at $36.5 billion. Within minutes of a stroke, the lack of blood flow will permanently damage a core of brain tissue. Between this damaged core and normal brain tissue is a region of tissue known as the penumbra—tissue that is under gradated stress from lessened blood flow and some disruption of energy metabolism. Over the first 24-48 hours following a stroke incident, the stress on neuronal and glia cells in the penumbra resolves either with some recovery or further cell death.

In one aspect, the present invention provides a method of neuroprotective therapy in a stroke patient, comprising administering to a patient in need thereof an effective amount of a compound described herein, or a pharmaceutically acceptable salt thereof or composition comprising the same. In some embodiments, such therapy salvages as much of the penumbra as possible, and/or limits further acute tissue damage, and/or promotes neuron recovery. In another aspect is provided a method of treating stroke or promoting recovery from stroke, comprising administering to a patient in need thereof an effective amount of a compound described herein, or a pharmaceutically acceptable salt thereof or composition comprising the same. In another aspect is provided a method of promoting or increasing neuroprotection, neuroregeneration, or neurorestoration in a patient who has suffered a stroke, comprising administering to the patient an effective amount of a compound described herein, or a pharmaceutically acceptable salt thereof or composition comprising the same. In another aspect is provided a method of treating stroke or promoting recovery from stroke, comprising administering to a patient in need thereof an effective amount of a compound described herein, or a pharmaceutically acceptable salt thereof or composition comprising the same, wherein the compound is an A₃R agonist. In another aspect is provided a method of treating stroke or promoting recovery from stroke, comprising administering to a patient in need thereof an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is one of those described in Table 1, or a pharmaceutically acceptable salt thereof or composition comprising the same.

In some embodiments, the stroke is selected from ischemic stroke, hemorrhagic stroke, subarachnoid hemorrhage, cerebral vasospasm, and transient ischemic attacks (TIA). In some embodiments, the stroke is ischemic, e.g., an acute ischemic stroke (AIS). In some embodiments, the stroke is hemorrhagic. In some embodiments, the compound is administered within 48 hours of the stroke. In some embodiments, the compound is administered within 24 hours of the stroke. In some embodiments, the compound is administered within 16 hours of the stroke. In some embodiments, the compound is administered within 8, 4, 2, or 1 hours of the stroke. In some embodiments, the compound is administered for at least the first 1-72 hours following the stroke. In some embodiments, the compound is administered for at least the first 8-52 hours following the stroke. In some embodiments, the compound is administered for at least the first 8-48 hours following the stroke. In some embodiments, the compound is administered for at least the first 24-48 hours following the stroke. In some embodiments, the compound is administered chronically to treat the stroke as it occurs. In some embodiments, the compound is administered chronically to treat Transient Ischemic Attacks (TIA).

In some embodiments, the compound is administered chronically to treat ischemic stroke, hemorrhagic stroke, a subarachnoid hemorrhage, cerebral vasospasm, transient ischemic attacks (TIA), or treat a patient who is at an increased risk for a stroke, such as a patient who has had a stroke in the past and is at risk for a further stroke, such as a patient over the age of 40, 45, 50, 55, 60, 65, 70, 75, or 80 years of age.

In some embodiments, the compound treats an ischemia-reperfusion injury caused by the stroke.

In certain embodiments, for treatment of stroke and related conditions, a recanalization procedure such as thrombolysis by recombinant tissue plasminogen activator (r-tPA) or mechanical thrombectomy is used in combination with a presently disclosed method of treating stroke or the related condition.

### Neurodegenerative Diseases

Neurodegenerative diseases are incurable, progressive, and ultimately debilitating syndromes resulting from the progressive degeneration and/or death of neurons in the brain and spinal cord. Neurodegeneration results in movement (ataxias) and/or cognitive function (dementias) disorders, and includes a spectrum of diseases such as Alzheimer's Disease (AD), Parkinson's Disease (PD), Huntington's Disease (HD), Multiple Sclerosis (MS), amyotrophic lateral sclerosis (ALS), and chronic traumatic encephalopathy (CTE). While many neurodegenerative diseases are principally genetic in origin, other causes can include viruses, alcoholism, tumors or toxins, and as is now clear, repetitive brain injuries.

Neurons accumulate cellular damage over time due to the foregoing factors, which is generally considered the reason why many neurodegenerative diseases associated with prolonged cellular stress, such as Alzheimer's disease and Parkinson's disease, occur in aged individuals. Dementias represent the predominant outcome of neurodegenerative diseases with AD representing approximately 60-70% of cases. (Kandale 2013) As discussed above, activation of neuroprotective and neurorestorative mechanisms can ameliorate the progression of one or more neurodegenerative diseases. Accordingly, in one aspect the present invention provides a method of treating a neurodegenerative disease or promoting recovery from a neurodegenerative disease, comprising administering to a patient in need thereof an effective amount of a compound described herein, or a pharmaceutically acceptable salt thereof or composition comprising the same.

In one aspect, the present invention provides a method of promoting neuroprotection or neurorestoration in a patient suffering from a neurodegenerative disease, comprising administering to the patient an effective amount of a compound described herein, or a pharmaceutically acceptable salt thereof or composition comprising the same. In some embodiments is provided a method of promoting neuroprotection or neurorestoration in a patient suffering from a neurodegenerative disease, comprising administering to the patient an effective amount of a compound described herein, or a pharmaceutically acceptable salt thereof or composition comprising the same, wherein the compound is an A₃R agonist. In some embodiments is provided a method of promoting neuroprotection or neurorestoration in a patient suffering from a neurodegenerative disease, comprising administering to the patient an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is a compound described in Table 1, or a pharmaceutically acceptable salt thereof or a composition comprising the same.

### Alzheimer's Disease (AD)

An estimated 5.2 million Americans of all ages had AD in 2014; 11% of the population age 65 and older have AD. (Alzheimer's Association) By 2050, the number of people age 65 and older with AD is projected to nearly triple to a projected 13.8 million. In the U.S., the cost of providing care for AD patients is about $214 billion per year; 70% of this cost is covered by Medicare and Medicaid. The current trends would project these costs to grow to $1.2 trillion per year by 2050.

Activation of astrocytes and promoting neuroprotection and neurorestoration according to the present invention represents a new treatment option for AD. Accordingly, provided herein in one aspect is a method of treating AD or promoting neuroprotection or neurorestoration in a patient suffering from AD, comprising administering to the patient an effective amount of a compound described herein, or a pharmaceutically acceptable salt thereof or composition comprising the same. In some embodiments, the present invention provides a method of treating AD or promoting neuroprotection or neurorecovery in a patient suffering from AD, comprising administering to the patient an effective amount of a compound described herein, or a pharmaceutically acceptable salt thereof or composition comprising the same, wherein the compound is an A₃R agonist. In some embodiments, the present invention provides a method of treating AD or promoting neuroprotection or neurorecovery in a patient suffering from AD, comprising administering to the patient an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is a compound described in Table 1, or a pharmaceutically acceptable salt thereof, or a composition comprising the same.

In some embodiments, beneficial effects resulting from a method of treating AD provided herein include, but are not limited to, one or more of: improving cognitive function, decreasing cognitive impairment, decreasing plaque burden, enhancing beta amyloid clearance, increasing synaptogenesis, and improving memory.

### Parkinson's Disease (PD)

As many as one million Americans live with PD, and each year approximately 60,000 Americans are newly diagnosed not including the thousands of cases that go undetected. (Parkinson's Disease Foundation) The total combined direct and indirect cost of PD, including medical treatment, social security payments and lost income, is estimated to be nearly $25 billion per year in the United States. (Parkinson's Disease Foundation and Huse 2005)

Activation of neuroprotection and neurorestoration according to the present invention represents a new treatment option for PD. Accordingly, provided herein in one aspect is a method of treating PD or promoting neuroprotection or neurorestoration in a patient suffering from PD, comprising administering to the patient an effective amount of a compound described herein, or a pharmaceutically acceptable salt thereof or composition comprising the same. In some embodiments, the present invention provides a method of treating PD or promoting neuroprotection or neurorecovery in a patient suffering from PD, comprising administering to the patient an effective amount of a compound described herein, or a pharmaceutically acceptable salt thereof or composition comprising the same, wherein the compound is an A₃R agonist. In some embodiments, the present invention provides a method of treating PD or promoting neuroprotection or neurorecovery in a patient suffering from PD, comprising administering to the patient an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is one of those described in Table 1, or a pharmaceutically acceptable salt thereof or a composition comprising the same.

### Multiple Sclerosis (MS)

More than 400,000 people in the United States have MS. In young adults, MS represents the most prevalent disease of the central nervous system. (Multiple Sclerosis Foundation) There is potential for astrocytes to reverse the destruction of nerve cell myelin coatings that is caused by MS by their neurorestorative effects and promotion of healing in the damaged CNS of MS patients.

Activation of neuroprotection and neurorestoration in the CNS according to the present invention thus represents a new treatment option for MS. Accordingly, provided herein in one aspect is a method of treating MS or promoting neuroprotection or neurorestoration in a patient suffering from MS, comprising administering to the patient an effective amount of a compound described herein, or a pharmaceutically acceptable salt thereof or composition comprising the same. In some embodiments, the present invention provides a method of treating MS or promoting neuroprotection or neurorecovery in a patient suffering from MS, comprising administering to the patient an effective amount of a compound described herein, or a pharmaceutically acceptable salt thereof or composition comprising the same, wherein the compound is an A₃R agonist. In some embodiments, the present invention provides a method of treating MS or promoting neuroprotection or neurorecovery in a patient suffering from MS, comprising administering to the patient an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is one of those described in Table 1, or a pharmaceutically acceptable salt thereof or a composition comprising the same.

### Amyotrophic Lateral Sclerosis (ALS) Lou Gehrig's Disease

Approximately 5,600 people in the U.S. are diagnosed with ALS each year; as many as 30,000 Americans may have the disease concurrently. (ALS Association) Activation of astrocytes can provide stimulation of recovery and repair of the neurons and their connections in an ALS patient.

Accordingly, provided herein in one aspect is a method of treating ALS or promoting neuroprotection or neurorestoration in a patient suffering from ALS, comprising administering to the patient an effective amount of a compound described herein, or a pharmaceutically acceptable salt thereof or composition comprising the same. Also provided in other embodiments is a method of stimulating recovery and repair of the neurons and their connections in an ALS patient, comprising administering to the patient an effective amount of a compound described herein, or a pharmaceutically acceptable salt thereof or composition comprising the same. In some embodiments, the present invention provides a method of treating ALS or promoting neuroprotection or neurorecovery in a patient suffering from ALS, comprising administering to the patient an effective amount of a compound described herein, or a pharmaceutically acceptable salt thereof or composition comprising the same, wherein the compound is an A₃R agonist. In some embodiments, the present invention provides a method of treating ALS or promoting neuroprotection or neurorecovery in a patient suffering from ALS, comprising administering to the patient an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is one of those described in Table 1, or a pharmaceutically acceptable salt thereof or a composition comprising the same.

### Chronic Traumatic Encephalopathy (CTE)

CTE (a form of tauopathy) is a progressive neurodegenerative disease found in individuals who have suffered one or more (often multiple or repeated over the course of time) severe blows to the head. CTE is most often diagnosed in professional athletes in American football, soccer, hockey, professional wrestling, stunt performing, bull riding and rodeo performing, motocross, and other contact sports who have experienced brain trauma and/or repeated concussions. A subset of CTE sufferers have chronic traumatic encephalomyopathy (CTEM), which is characterized by motor neuron disease symptoms that mimic ALS. Progressive muscle weakness and motor and gait abnormalities are believed to be early signs of CTEM. First stage symptoms of CTE include progressive attention deficit, disorientation, dizziness, and headaches. Second stage symptoms comprise memory loss, social instability, erratic behavior, and poor judgment. In third and fourth stages, patients suffer progressive dementia, slowed movements, tremors, hypomimia, vertigo, speech impediments, hearing loss, and suicidality, and may further include dysarthria, dysphagia, and ocular abnormalities, e.g., ptosis.

Accordingly, provided herein in one aspect is a method of treating or preventing CTE or promoting neuroprotection or neurorestoration in a patient suffering from CTE, comprising administering to the patient an effective amount of a compound described herein, or a pharmaceutically acceptable salt thereof or composition comprising the same. Also provided in other embodiments is a method of stimulating recovery and repair of the neurons and their connections in a CTE patient, comprising administering to the patient an effective amount of a compound described herein, or a pharmaceutically acceptable salt thereof or composition comprising the same. In some embodiments, the compound treats one or more symptoms of first stage, second stage, third stage, or fourth stage CTE. In some embodiments, the present invention provides a method of treating CTE or promoting neuroprotection or neurorecovery in a patient suffering from CTE, comprising administering to the patient an effective amount of a compound described herein, or a pharmaceutically acceptable salt thereof or composition comprising the same, wherein the compound is an A₃R agonist. In some embodiments, the present invention provides a method of treating CTE or promoting neuroprotection or neurorecovery in a patient suffering from CTE, comprising administering to the patient an effective amount of a compound described herein, or a pharmaceutically acceptable salt thereof or composition comprising the same, wherein the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound acts as an agonist of an A₁ adenosine receptor (A₁R). In some embodiments, the compound is one of those described in Table 1, or a pharmaceutically acceptable salt thereof or a composition comprising the same.

On a microscopic scale the pathology includes neuronal death, tau deposition, TAR DNA-binding Protein 43 (TDP 43) beta-amyloid deposition, white matter changes, and other abnormalities. Tau deposition includes the increasing presence of dense neurofibrillary tangles (NFT), neurites, and glial tangles, which are made up of astrocytes and other glial cells. Thus, in some embodiments, the method treats, enhances clearance or prevents neuronal death, tau deposition, TAR DNA-binding Protein 43 (TDP 43) beta-amyloid deposition, white matter changes, and other abnormalities associated with CTE.

In some embodiments, the present invention provides long-term administration of a compound disclosed herein, such as a biased agonist, partial agonist, or biased partial agonist of A₃R, or a dual agonist at an A₃R and an A₁R, or a biased agonist, partial agonist, or biased partial agonist of P2Y₁, to treat a neurodegenerative disease, such as one of those described herein. In some embodiments, the present invention provides long-term administration of a compound disclosed herein, such as a biased agonist, partial agonist, or biased partial agonist of A₁R, to treat a neurodegenerative disease, such as one of those described herein.

### Cardiovascular Diseases

Disclosed compounds are also useful in treating a variety of cardiovascular diseases and conditions. In some embodiments, the present invention provides a method of treating a heart (cardiac) or cardiovascular disease, such as cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, or atherosclerosis, comprising administering an effective amount of a compound described herein, or a pharmaceutically acceptable salt thereof or composition comprising the same. In some embodiments, a disclosed compound modulates ATP-sensitive potassium channels, for example via biased agonism, partial agonism, or biased partial agonism at an A₃R receptor, or dual agonism at an A₃R and an A₁R. In some embodiments, a disclosed compound modulates ATP-sensitive potassium channels via biased agonism, partial agonism, or biased partial agonism at an A₁R receptor.

In some embodiments, the heart or cardiovascular disease is cardiac ischemia or myocardial infarction.

In some embodiments, the present invention provides a method of promoting or increasing cardioprotection, cardiorestoration, or cardioregeneration in a patient suffering from a heart (cardiac) or cardiovascular disease or condition, comprising administering to the patient an effective amount of a disclosed compound, for example one of those described in Table 1, or a pharmaceutically acceptable salt thereof or a composition comprising the same.

In some embodiments, the heart (cardiac) or cardiovascular disease from which the patient is suffering is cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, or atherosclerosis.

In some embodiments, the compound acts as an agonist of an A₃ adenosine receptor (A₃R). In some embodiments, the compound acts as a dual agonist of an A₃R and an A₁ adenosine receptor (A₁R). In some embodiments, the compound acts as an agonist of an A₁R.

### Other Diseases

Compounds that modulate beneficial effects such as neuroprotection, for example by increasing astrocyte mitochondrial activity, also have the potential to treat a variety of other diseases. For example, due to the role of astrocytes in neuroprotection disclosed in the present invention, activation of astrocytes, for example via modulation of A₃R and/or A₁R, is useful in treating various diseases and conditions discussed below.

Accordingly, in some embodiments, the present invention provides a method of treating neurodegeneration in a patient suffering from a disease or condition, comprising administering to the patient an effective amount of a compound described herein, or a pharmaceutically acceptable salt thereof or composition comprising the same.

In some embodiments, the present invention provides a method of promoting or increasing neuroprotection, neurorestoration, or neuroregeneration in a patient suffering from a disease or condition, comprising administering to the patient an effective amount of a compound described herein, or a pharmaceutically acceptable salt thereof or composition comprising the same.

In some embodiments, the disease or condition is selected from autoimmune diseases, allergic diseases, and/or transplant rejection and graft-versus-host disease (for the use of certain nucleoside and nucleotide compounds in treating these conditions, see, for example, WO 2007/20018, hereby incorporated by reference). In other embodiments, the disease or condition is selected from intraocular hypertension and/or glaucoma (for the use of certain nucleoside and nucleotide compounds in treating these conditions, see, for example, WO 2011/77435, hereby incorporated by reference). In other embodiments, the disease or condition is selected from odor sensitivity and/or an olfactory disorder (for the use of certain nucleoside and nucleotide compounds in treating these conditions, see, for example, EP1624753, hereby incorporated by reference). In other embodiments, the disease or condition is type 2 diabetes (for the use of certain nucleoside and nucleotide compounds in treating these conditions, see, for example, US 2010/0256086, hereby incorporated by reference).

In other embodiments, the disease or condition is selected from respiratory diseases and/or cardiovascular (CV) diseases (for the use of certain nucleoside and nucleotide compounds in treating these conditions, see, for example, FASEB J. (2013) 27:1118.4 (abstract of meeting), hereby incorporated by reference). In other embodiments, the disease or condition is selected from deficits in CNS function, deficits in learning and/or deficits in cognition (for the use of certain nucleoside and nucleotide compounds in treating these conditions, see, for example, Neuropsychopharmacology 2015 January; 40(2):305-14. doi: 10.1038/npp.2014.173. Epub 2014 Jul. 15. “Impaired cognition after stimulation of a P2Yᵢ receptor in the rat medial prefrontal cortex,” Koch, H. et al. PMID: 25027332, hereby incorporated by reference). In other embodiments, the disease or condition is selected from a neurodegenerative disease such as Alzheimer's disease, Parkinson's disease, Huntington's disease, prion disease, and/or amyotrophic lateral sclerosis (for the use of certain nucleoside and nucleotide compounds in treating these conditions, see, for example, U.S. Pat. No. 8,691,775, hereby incorporated by reference). In other embodiments, the disease or condition is selected from otic disorders, Meniere's disease, endolymphatic hydrops, progressive hearing loss, dizziness, vertigo, tinnitus, collateral brain damage associated with radiation cancer therapy, and/or migraine treatment (for the use of certain nucleoside and nucleotide compounds in treating these conditions, see, for example, US 2009/0306225; UY31779; and U.S. Pat. No. 8,399,018, each of which is hereby incorporated by reference). In other embodiments, the disease or condition is selected from pathological sleep perturbations, depression, sleep disorders in the elderly, Parkinson's disease, Alzheimer's disease, epilepsy, schizophrenia, and/or symptoms experienced by recovering alcoholics (for the use of certain nucleoside and nucleotide compounds in treating these conditions, see, for example, US 2014/0241990, hereby incorporated by reference). In other embodiments, the disease or condition is selected from damage to neurons or nerves of the peripheral nervous system during surgery (for the use of certain nucleoside and nucleotide compounds in treating these conditions, see, for example, U.S. Pat. No. 8,685,372, hereby incorporated by reference). In other embodiments, the disease or condition is a cancer such as prostate cancer (for the use of certain nucleoside and nucleotide compounds in treating these conditions, see, for example, Biochem Pharmacol. 2011 Aug. 15; 82(4): 418-425. doi:10.1016/j.bcp.2011.05.013. “Activation of the P2Y1 Receptor Induces Apoptosis and Inhibits Proliferation of Prostate Cancer Cells,” Qiang Wei et al., hereby incorporated by reference). In other embodiments, the disease or condition is selected from one or more gastrointestinal conditions such as constipation and/or diarrhea (for the use of certain nucleoside and nucleotide compounds in treating these conditions, see, for example, Acta Physiol (Oxf). 2014 December; 212(4):293-305. doi: 10.1111/apha.12408. “Differential functional role of purinergic and nitrergic inhibitory cotransmitters in human colonic relaxation,” Mane N1, Gil V, Martinez-Cutillas M, Clave P, Gallego D, Jimenez M.; and Neurogastroenterol. Motil. 2014 January; 26(1):115-23. doi: 10.1111/nmo.12240. Epub 2013 Oct. 8. “Calcium responses in subserosal interstitial cells of the guinea-pig proximal colon,” Tamada H., Hashitani H. PMID: 24329947, hereby incorporated by reference).

In other embodiments, the disease or condition is selected from cancer of the brain, such as glioblastoma (for the use of certain nucleoside and nucleotide compounds in treating these conditions, see, for example, Purinergic Signal. 2015 September; 11(3):331-46. doi: 10.1007/s11302-015-9454-7. Epub 2015 May 15. “Potentiation of temozolomide antitumor effect by purine receptor ligands able to restrain the in vitro growth of human glioblastoma stem cells.” D′Alimonte, I. et al. PMID: 25976165, hereby incorporated by reference). In other embodiments, the disease or condition is selected from a gastrointestinal disorder such as diarrhea (for the use of certain nucleoside and nucleotide compounds in treating these conditions, see, for example, Acta Physiol (Oxf). 2014 December; 212(4):293-305. doi: 10.1111/apha.12408. “Differential functional role of purinergic and nitrergic inhibitory cotransmitters in human colonic relaxation,” Mañe N., Gil V, Martinez-Cutillas M, Clavé P, Gallego D, Jiménez M., hereby incorporated by reference). In other embodiments, the disease or condition is impaired cognition (for the use of certain nucleoside and nucleotide compounds in treating this condition, see, for example, Neuropsychopharmacology. 2015 January; 40(2):305-14. doi: 10.1038/npp.2014.173. Epub 2014 Jul. 15. “Impaired cognition after stimulation of P2Y₁ receptors in the rat medial prefrontal cortex,” Koch H, Bespalov A, Drescher K, Franke H, Krügel U. PMID: 25027332, hereby incorporated by reference).

In some embodiments, the present invention provides a method of treating a disease or condition associated with brain injury or a neurodegenerative condition, such as epilepsy, migraine, collateral brain damage associated with radiation cancer therapy, depression, mood or behavioral changes, dementia, erratic behavior, suicidality, tremors, Huntington's chorea, loss of coordination of movement, deafness, impaired speech, dry eyes, hypomimia, attention deficit, memory loss, cognitive difficulties, vertigo, dysarthria, dysphagia, ocular abnormalities, or disorientation, comprising administering to a patient in need thereof an effective amount of a disclosed compound. In some embodiments, the compound is an A₃R agonist. In some embodiments, the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound is a biased agonist, partial agonist, or biased partial agonist or antagonist at an A₁ receptor. In some embodiments, the compound is one of those described in Table 1, or a pharmaceutically acceptable salt thereof or a composition comprising the same.

In further embodiments, the present invention provides a method of treating a neurodegenerative disease selected from the group consisting of Alzheimer's disease, Parkinson's disease, Huntington's disease, multiple sclerosis, amyotrophic lateral sclerosis, and prion disease in a patient in need thereof, comprising administering an effective amount of a compound described herein, or a pharmaceutically acceptable salt thereof or composition comprising the same. In some embodiments, the compound is an A₃R agonist. In some embodiments, the compound is an A₁R agonist. In some embodiments, the compound is a biased agonist, partial agonist, or biased partial agonist or antagonist at an A₃ receptor. In some embodiments, the compound acts by dual agonism at an A₃R and an A₁R. In some embodiments, the compound is a biased agonist, partial agonist, or biased partial agonist or antagonist at an A₁ receptor. In some embodiments, the compound is one of those described in Table 1, or a pharmaceutically acceptable salt thereof or a composition comprising the same.

In some embodiments, the improvement in cognitive or neurological function is measured as a score increase between about 1% and 20% in the delayed verbal recall task of the revised Wechsler Memory Scale. For example, the improvement in cognitive function may be measured as a score increase between about 1% and 10%, or between about 1% and 5%, or between about 5% and 15%.

In some embodiments, the present invention provides a method of treating a brain or central nervous system (CNS) injury or condition selected from traumatic brain injury (TBI) or stroke, comprising administering to a patient in need thereof an effective amount of a compound described herein, or a pharmaceutically acceptable salt thereof or composition comprising the same.

In some embodiments, the brain or central nervous system (CNS) injury or condition is TBI. In some embodiments, the TBI is selected from concussion, blast injury, combat-related injury, or a mild, moderate or severe blow to the head.

In some embodiments, the compound described herein, or a pharmaceutically acceptable salt thereof or composition comprising the same, is administered within 24 hours of the TBI or stroke.

In some embodiments, the compound described herein, or a pharmaceutically acceptable salt thereof or composition comprising the same, is administered within 8 hours of the TBI or stroke.

In some embodiments, the compound described herein, or a pharmaceutically acceptable salt thereof or composition comprising the same, is administered at least during the first 8-48 hours following the TBI or stroke.

In some embodiments, the brain or central nervous system (CNS) injury or condition is stroke.

In some embodiments, the compound described herein, or a pharmaceutically acceptable salt thereof or composition comprising the same, is administered chronically to treat the stroke during the time period after the stroke has occurred as it resolves.

In some embodiments, neuroprotection or neurorestoration is increased in the patient as compared with an untreated patient.

In some embodiments, the compound is a biased partial agonist at a human A₃ adenosine receptor (A₃R). In some embodiments, the compound acts by dual agonism at an A₃R and an A₁R. In some embodiments, the compound is a biased partial agonist at a human A₁ adenosine receptor (A₁R).

In some embodiments, the A₃R is partially agonized in a manner biased toward neuroprotective functions of the A₃R receptor.

In some embodiments, the compound described herein, or a pharmaceutically acceptable salt thereof or composition comprising the same, is administered orally, intravenously, or parenterally.

In one aspect, the present invention provides a method of increasing neuroprotection or neurorestoration in a patient who has suffered a TBI or stroke, comprising administering to a patient in need thereof an effective amount of a compound described herein, or a pharmaceutically acceptable salt thereof or composition comprising the same.

In some embodiments, the neuroprotection or neurorestoration decreases the recovery period after the TBI or stroke as compared with an untreated patient.

In some embodiments, the compound is a biased partial agonist at a human A₃ adenosine receptor (A₃R) and the A₃R is partially agonized in a manner biased toward neuroprotective functions of the A₃R receptor. In some embodiments, the compound acts by dual agonism at an A₃R and an A₁R. In some embodiments, the compound is a biased partial agonist at a human A₁ adenosine receptor (A₁R) and the A₁R is partially agonized in a manner biased toward neuroprotective functions of the A₁R receptor. In some embodiments, the compound acts as an agonist at an A₁R.

In some embodiments, the compound described herein, or a pharmaceutically acceptable salt thereof or composition comprising the same, is administered orally, intravenously, or parenterally.

In one aspect, the present invention provides a method of treating an injury, disease, or condition selected from traumatic brain injury (TBI), stroke, a neurodegenerative condition, or a heart or cardiovascular disease, comprising administering to a patient in need thereof an effective amount of a compound described herein, or a pharmaceutically acceptable salt thereof or composition comprising the same.

In some embodiments, the injury, disease, or condition is TBI. In some embodiments, the TBI is selected from concussion, blast injury, combat-related injury, or a mild, moderate or severe blow to the head.

In some embodiments, the injury, disease, or condition is a stroke selected from ischemic stroke, hemorrhagic stroke, subarachnoid hemorrhage, cerebral vasospasm, or transient ischemic attacks (TIA).

In some embodiments, the neurodegenerative disease is selected from Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), or a neurodegenerative condition caused by a virus, alcoholism, tumor, toxin, or repetitive brain injuries.

In some embodiments, the injury, disease, or condition is Parkinson's Disease.

In some embodiments, the injury, disease, or condition is Alzheimer's Disease, migraine, brain surgery, or a neurological side effect associated with cancer chemotherapy.

In some embodiments, the heart or cardiovascular disease is selected from cardiac ischemia, myocardial infarction, a cardiomyopathy, coronary artery disease, arrhythmia, myocarditis, pericarditis, angina, hypertensive heart disease, endocarditis, rheumatic heart disease, congenital heart disease, or atherosclerosis.

In some embodiments, the heart or cardiovascular disease is cardiac ischemia or myocardial infarction.

In some embodiments, the compound described herein, or a pharmaceutically acceptable salt thereof or composition comprising the same, is administered chronically to treat the stroke, cardiac ischemia, or myocardial infarction during the time period after the injury has occurred as it resolves.

In some embodiments, neuroprotection or neurorestoration is increased in the patient as compared with an untreated patient.

In some embodiments, the A₃R is agonized in a biased manner toward neuroprotective functions of the A₃R receptor via preferential activation of intracellular calcium mobilization with less, or no, activation of other A₃R-mediated pathways, or via preferential activation of Gq11-mediated intracellular calcium mobilization, Gi-mediated modulation of cAMP production, or Gi-mediated phosphorylation of ERK1/2 and Akt.

In some embodiments, the A₃R is partially agonized in a manner biased toward cardioprotective functions of the A₃R receptor via preferential activation of intracellular calcium mobilization with less, or no, activation of other A₃R-mediated pathways, or via preferential activation of Gq11-mediated intracellular calcium mobilization, Gi-mediated modulation of cAMP production, or Gi-mediated phosphorylation of ERK1/2 and Akt.

In some embodiments, the method increases neuroprotection or neurorestoration in a patient who is suffering from a neurological side effect associated with or resulting from cancer chemotherapy or brain surgery.

In some embodiments, the compound described herein, or a pharmaceutically acceptable salt thereof or composition comprising the same, is administered orally.

In one aspect, the present invention provides a method of increasing neuroprotection or neurorestoration in a patient who has suffered a TBI or stroke, thereby treating the TBI or stroke, comprising administering to a patient in need thereof an effective amount of a compound described herein, or a pharmaceutically acceptable salt thereof or composition comprising the same.

In one aspect, the present invention provides a method of increasing cardioprotection or regeneration of damaged heart tissue in a patient who has suffered a cardiac ischemia or myocardial infarction, thereby treating the cardiac ischemia or myocardial infarction, comprising administering to a patient in need thereof an effective amount of a compound described herein, or a pharmaceutically acceptable salt thereof or composition comprising the same.

In some embodiments, the recovery period after the TBI, stroke, cardiac ischemia, or myocardial infarction is decreased as compared with an untreated patient.

In some embodiments, the A₃R is partially agonized in a manner biased toward neuroprotective functions of the A₃R receptor.

In some embodiments, the A₃R is partially agonized in a manner biased toward cardioprotective functions of the A₃R receptor.

In some embodiments, the compound described herein, or a pharmaceutically acceptable salt thereof or composition comprising the same, is administered orally.

In some embodiments, the compound is a biased agonist of an A₃R with improved cardioprotection function relative to a full A₃R agonist.

In some embodiments, the compound is a biased agonist of an A₃R with improved cardioprotection function relative to a full A₃R agonist via preferential activation of one or more of the following A₃R-mediated pathways: activation of Gq11-mediated intracellular calcium mobilization, Gi-mediated modulation of cAMP production, Gi-mediated phosphorylation of ERK1/2 and Akt, or modulation of Beta-Arrestin activation.

In some embodiments, the compound is a biased agonist of an A₃R with improved cardioprotection function relative to a full A₃R agonist via preferential activation of intracellular calcium mobilization with less or no activation of the other A₃R-mediated pathways.

In some embodiments, the compound is a partial agonist of the A₃R with improved cardioprotection function relative to a full A₃R agonist.

### Addictive Disorders

Disclosed compounds are also useful in treating addictions, addictive behaviors, behavioral addictions, compulsive disorders and behaviors, and related conditions.

The use of certain compounds in treating such addictions, behaviors, and disorders is described in WO/2019/157317, the contents of which are hereby incorporated by reference.

Cocaine self-administering mice exhibit significantly higher glutamate levels in the VTA (ventral tegmental area) of the brain. The VTA, in particular the VTA dopamine neurons, serve several functions in the reward system, motivation, cognition, and drug addiction, and may be the focus of several psychiatric disorders. The elevated glutamate levels appear to be due, at least in part, to loss of glutamate uptake into astrocytes. Without wishing to be bound by theory, it is believed that reduced availability of glutamate has negative effects on astrocyte function and this loss of function affects neuronal activity and drug-seeking behavior. It has now been found that the compounds disclosed herein treat or prevent relapse in addicted individuals, for example by reversing such loss of astrocyte function. Such loss of astrocyte function may be partly due to reduced expression of the glutamate transporter (GLT-1) in astrocytes. Since astrocytes metabolize glutamate to produce ATP, this likely impairs glutamate uptake, weakens astrocyte oxidative metabolism and downstream ATP-dependent processes and thereby weakens their ability to maintain an optimal environment for VTA neuronal activity.

Accordingly, in one aspect, the present invention provides a method of preventing, ameliorating, treating, or promoting recovery from an addiction, addictive behavior, behavioral addiction, brain reward system disorder, compulsive disorder, or related condition, comprising administering to a subject in need thereof an effective amount of a compound described herein, or a pharmaceutically acceptable salt thereof or composition comprising the same.

In some embodiments, the addiction is to an addictive substance. In some embodiments, the addictive substance is a prescription or recreational drug.

In some embodiments, the addictive substance is selected from alcohol, nicotine, a stimulant, a cannabinoid agonist, or an opioid agonist. In some embodiments, the addictive substance is selected from heroin, cocaine, alcohol, an inhalant, an opioid, nicotine, an amphetamine, or a synthetic analog, salt, composition, or combination thereof.

In some embodiments, the amphetamine is selected from bupropion, cathinone, MDMA, or methamphetamine.

In some embodiments, the prescription or recreational drug is selected from a cannabinoid agonist or opioid agonist.

In some embodiments, the addiction is an alcohol or nicotine addiction.

In some embodiments, the subject is a polydrug abuser.

In some embodiments, the prescription or recreational drug is selected from cocaine, heroin, bupropion, cathinone, MDMA, or methamphetamine morphine, oxycodone, hydromorphone, fentanyl, or a combination thereof.

In some embodiments, a disclosed compound increases energy metabolism mediated by astrocytes, such as astrocyte mitochondria. In some embodiments, the compound reverses loss of glutamate uptake into astrocytes caused by a substance with abuse potential. In some embodiments, the compound at least partially reverses the remodeling of the brain reward system caused by the addiction. In some embodiments, such effects are mediated by brain or CNS adenosine A₃ receptors, such as astrocyte A₃R in the VTA; or microglia A₃R.

In another aspect, the present invention provides a method of preventing, ameliorating, treating, or promoting recovery from an addiction, addictive behavior, behavioral addiction brain reward system disorder, compulsive disorder, or related condition by increasing energy metabolism mediated by astrocytes, glia, microglia, neurons, endothelium cells, or other cells of the brain and/or CNS, comprising administering to a subject in need thereof an effective amount of a compound described herein, or a pharmaceutically acceptable salt thereof or composition comprising the same.

In some embodiments, the method treats or prevents a relapse of an addiction or addictive behavior in the subject. In some embodiments, the subject is addicted to one or more addictive substances such as addictive drugs (drugs having abuse potential). As described below, such drugs include prescription drugs and recreational drugs such as heroin, cocaine, nicotine, or an opioid agonist.

In another aspect, the present invention provides a method of treating or preventing withdrawal caused by addiction to one or more addictive substances or drugs, comprising administering to a subject in need thereof an effective amount of a compound described herein, or a pharmaceutically acceptable salt thereof or composition comprising the same. In some embodiments, the compound decreases withdrawal symptoms in an addicted individual in withdrawal. In some embodiments, the compound treats withdrawal in an addicted individual in withdrawal. In some embodiments, the method further comprises co-administering another drug for treating withdrawal and, optionally, counseling such as psychotherapy. In some embodiments, the method further comprises a cognitive behavioral therapy. In some embodiments, the method further comprises a digital therapeutic. Digital therapeutics include, for example, reSET or reSET-O (Pear Therapeutics).

In some embodiments, the present invention provides a method of treating or preventing a relapse of a compulsive disorder or compulsive behavior, comprising administering to a subject in need thereof an effective amount of a compound described herein, or a pharmaceutically acceptable salt thereof or composition comprising the same.

In some embodiments, the compulsive disorder is obsessive-compulsive disorder (OCD), Tourette syndrome, trichotillomania, anorexia, bulimia, anxiety disorder, psychosis, or post-traumatic stress disorder.

According to another aspect, the present invention provides a method for treating one or more behavioral addictions and addictive behaviors or disorders comprising administering to a subject in need thereof a compound described herein, or a pharmaceutically acceptable salt thereof or composition comprising the same. Behavioral addictions and addictive disorders result from the intoxication one senses from the release of brain chemicals (e.g., serotonin, adrenaline, epinephrine, etc.) during certain activities. Such disorders are known in the art and include gambling, sex addiction, pornography addiction, eating disorders, spending addiction, rage/anger, workaholism, exercise addiction, risk taking addictions (e.g. kleptomania and pyromania), perfectionism, internet or video game addiction, and compulsive use of electronic devices such as texting and checking social media, to name a few.

In some embodiments, activation of astrocytes is achieved through contacting with a disclosed compound one or more purinergic receptors such as adenosine receptors (ARs), for example those associated with or expressed by astrocytes or microglia, thus modulating the activity of the one or more receptors. In some embodiments, through effects on adenosine receptors such as A₁, A_{2A}, A_{2B} and A₃ on astrocytes, the compound activates astrocytes to treat one or more disclosed diseases or conditions. In some embodiments, after administration to a subject in need thereof, a disclosed compound influences one or more functions such as glutamate uptake having an impact on energy metabolism of astrocytes or neuronal function, thus treating one or more diseases or conditions. In some embodiments, the compound is an AR agonist. In some embodiments, the purinergic receptor is an adenosine A₃ receptor (A₃R). In some embodiments, the compound is an A₃R agonist. In some embodiments, the compound is a partial agonist or biased agonist or biased partial agonist, at an A₃ receptor (A₃R), such as a human A₃ receptor (hA₃R). In some embodiments, the compound is a biased antagonist at an A₃ receptor. In some embodiments, the compound acts by dual agonism at an A₃R and an A₁R. In some embodiments, the compound is an A₁R agonist. In some embodiments, the compound is one of those described in Table 1, or a pharmaceutically acceptable salt thereof or a composition comprising the same.

As used herein, the term “addiction” includes, unless otherwise specified, physical or psychological dependence on a substance. Addiction may involve withdrawal symptoms or mental or physical distress if the substance is withdrawn. Addiction includes drug liking, drug dependence, habit-formation, neurological and/or synaptic changes, development of brain reward system disorders, behavioral changes, or other signs or symptoms of addiction in a subject.

As used herein, the term “addictive drug” or “drug having abuse potential” includes drugs and other substances such as nicotine, whether approved by a regulatory body for treatment of a disease or not, that are known to result in clinical, behavioral, or neurological manifestations of addiction or compulsive behavior. In some embodiments, the addictive drug includes nicotine, a cannabinoid agonist, a stimulant, or an opioid agonist. “Addictive substance” refers to addictive drugs as well as other substances of abuse such as alcohol. Examples of addictive substances thus include heroin, cocaine, alcohol, opiates, nicotine, inhalants, amphetamines, and their synthetic analogs.

### Pain Conditions and Disorders

Disclosed compounds are also useful in treating pain, pain disorders, and related conditions. Accordingly, in one aspect, the present invention provides a method of treating, preventing, promoting recovery from, or ameliorating a pain condition or disorder, comprising administering to a subject in need thereof an effective amount of a compound described herein, or a pharmaceutically acceptable salt thereof or pharmaceutical composition thereof. In some embodiments, the compound is one of those described in Table 1, or a pharmaceutically acceptable salt thereof.

In some embodiments, the pain condition or disorder is pain control (pain management, e.g., management of chronic pain). For the use of certain nucleoside and nucleotide compounds in treating this and related conditions, see, for example, US 2010/0256086, hereby incorporated by reference.

In other embodiments, the pain condition or disorder is selected from pain mediated by the CNS, such as neuropathic pain, inflammatory pain, or acute pain. For the use of certain nucleoside and nucleotide compounds in treating these conditions, see, for example, Br J Pharmacol. 2010 March; 159(5):1106-17. doi: 10.1111/j.1476-5381.2009.00596.x. Epub 2010 Feb. 5. “A comparative analysis of the activity of ligands acting at P2X and P2Y receptor subtypes in models of neuropathic, acute and inflammatory pain.” Andó RD1, Méhész B, Gyires K, Illes P, Sperlágh B. PMID: 20136836, hereby incorporated by reference.

In some embodiments, the pain condition or disorder is migraine.

In some embodiments, the pain condition or disorder is neuropathic pain, inflammatory pain, or acute pain. See, e.g., Tosh, D. K.; Padia, J.; Salvemini, D.; Jacobson, K. A. Efficient, large-scale synthesis and preclinical studies of MRS5698, a highly selective A₃ adenosine receptor agonist that protects against chronic neuropathic pain. Purinergic Signalling 2015, 11, 371-387.

In some embodiments, the pain condition or disorder is central pain syndrome, peripheral neuropathy, corneal neuropathic pain, post stroke pain, or pain caused by multiple sclerosis.

In another aspect, the present invention provides a method of treating pain, comprising administering to a subject in need thereof an effective amount of a disclosed compound, such as I-1, or a pharmaceutically acceptable salt thereof or a pharmaceutically acceptable composition thereof.

In some embodiments, the pain is neuropathic pain. In some embodiments, the pain is inflammatory pain. In some embodiments, the pain is acute pain. In some embodiments, the pain is chronic pain. In some embodiments, the pain is nociceptive pain. In some embodiments, the pain is non-inflammatory musculoskeletal pain, fibromyalgia syndrome (FMS), or myofascial pain syndrome (MPS).

In some embodiments, the pain is selected from musculoskeletal pain, fibromyalgia, myofascial pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, chronic pain syndrome (CPS), central pain, trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiencies or alcoholism, acute pain after injuries, postoperative pain, pain during acute gout, and pain from an operation.

In some embodiments, the musculoskeletal pain is neck and shoulder pain and/or spasms, back pain, sciatica, chest ache, or thigh muscle ache.

In some embodiments, the pain is, or is associated with, otitis externa (OE), otitis media (OM), mastoiditis, bullous myringitis, eustachian tubal catarrh, labyrinthitis, facial nerve neuritis, temporal bone osteoradionecrosis, mal de debarquement, temporal bone fracture, or temporomandibular joint disease.

### Pharmaceutically Acceptable Compositions

According to another embodiment, the invention provides a composition comprising a disclosed compound and a pharmaceutically acceptable carrier, adjuvant, or vehicle. In certain embodiments, a composition of this invention is formulated for administration to a patient in need of such composition. In some embodiments, a composition of this invention is formulated for oral administration to a patient.

The term “biological sample,” as used herein, includes, without limitation, cell cultures or extracts thereof, biopsied material obtained from a mammal or extracts thereof, and blood, saliva, urine, feces, semen, tears, CSF, or other body fluids or extracts thereof.

The term “subject” or “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in unit dosage form for ease of administration and uniformity of dosage. The expression “unit dosage form,” as used herein, refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention are administered orally or parenterally at dosage levels of about 0.01 mg/kg to about 50 mg/kg and preferably from about 0.01 mg/kg to about 25 mg/kg, of subject body weight per day, one or more times a day, to obtain the desired therapeutic effect. In certain embodiments, the compounds of the invention are administered orally or parenterally at dosage levels of about 0.01 mg/kg to about 50 mg/kg, or about 0.01 mg/kg to about 25 mg/kg, or about 0.05 mg/kg to about 10 mg/kg, or about 0.05 mg/kg to about 5 mg/kg, or about 0.1 mg/kg to about 2.5 mg/kg, of subject body weight per day, one or more times a day, to obtain the desired therapeutic effect.

Liquid dosage forms for oral administration include, but are not limited to, pharmaceutically acceptable emulsions, liposomes, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters), poly(anhydrides) and cyclodextrins and modified cyclodextrins (such as SBE-bCD). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the invention can also be administered topically, such as directly to the eye, e.g., as an eye-drop or ophthalmic ointment. Eye drops typically comprise an effective amount of at least one compound of the invention and a carrier capable of being safely applied to an eye. For example, the eye drops are in the form of an isotonic solution, and the pH of the solution is adjusted so that there is no irritation of the eye. In many instances, the epithelial barrier interferes with penetration of molecules into the eye. Thus, most currently used ophthalmic drugs are supplemented with some form of penetration enhancer. These penetration enhancers work by loosening the tight junctions of the most superior epithelial cells (Burstein, 1985, Trans Ophthalmol Soc U K 104(Pt 4): 402-9; Ashton et al., 1991, J Pharmacol Exp Ther 259(2): 719-24; Green et al., 1971, Am J Ophthalmol 72(5): 897-905). The most commonly used penetration enhancer is benzalkonium chloride (Tang et al., 1994, J Pharm Sci 83(1): 85-90; Burstein et al, 1980, Invest Ophthalmol Vis Sci 19(3): 308-13), which also works as preservative against microbial contamination. It is typically added to a final concentration of 0.01−0.05%.

Combinations with Other Therapeutic Agents

Depending upon the particular condition to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.” Furthermore, standard of care treatments, including surgeries or use of medical devices, may be added advantageously to the methods of treatment described herein.

In certain embodiments, a provided compound, or composition thereof, is administered in combination with other therapeutic agents, such as tissue plasminogen activators, blood thinners, statins, ACE inhibitors, angiotensin II receptor blockers (ARBs), beta blockers, calcium channel blockers or diuretics, to a patient in need thereof.

In certain embodiments, the tissue plasminogen activator used in combination with compounds or compositions of the invention include, but are not limited to, alteplase, desmoteplase, reteplase, tenecteplase, or combinations of any of the above.

In certain embodiments, for treatment of stroke and related conditions, a recanalization procedure such as thrombolysis by recombinant tissue plasminogen activator (r-tPA) or mechanical thrombectomy is used in combination with a presently disclosed method of treating stroke or the related condition.

In certain embodiments, the blood thinners used in combination with compounds or compositions of the invention include, but are not limited to, warfarin, heparin, apixabam, clopidogrel, aspirin, rivaroxaban, dabigatran, or combinations of any of the above.

In certain embodiments, the statins used in combination with compounds or compositions of the invention include, but are not limited to, atorvastatin, rosuvastatin, fluvastatin, lovastatin, pravastatin, simvastatin and pitavastatin, cerivastatin, mevastatin, or combinations of any of the above.

In certain embodiments, the ACE inhibitors used in combination with compounds or compositions of the invention include, but are not limited to, captopril, enalapril, fosinopril, lisinopril, moexipril, perindopril, quinapril, ramipril, trandolapril benazepril, or combinations of any of the above.

In certain embodiments, the angiotensin II receptor blockers (ARBs) used in combination with compounds or compositions of the invention include, but are not limited to, azilsartan, candesartan, eprosartan, irbesartan, losartan, olmesartan, telmisartan, valsartan, fimasartan, or combinations of any of the above.

In certain embodiments, the beta blockers used in combination with compounds or compositions of the invention include, but are not limited to, atenolol, bisoprolol, betaxolol, carteolol, carvedilol, labetalol, metoprolol, nadolol, nebivolol, oxprenolol, penbutolol, pindolol, propranolol, timolol, or combinations of any of the above.

In certain embodiments, the calcium channel blockers used in combination with compounds or compositions of the invention include, but are not limited to, dihydropyridines: amlodipine, cilnidipine, clevidipine, felodipine, isradipine, lercanidipine, levamlodipine, nicardipine, nifedipine, nimodipine, nisoldipine, nitrendipine, diltiazem, verapamil, or combinations of any of the above.

In certain embodiments, the diuretics used in combination with compounds or compositions of the invention include, but are not limited to, loop diuretics, thiazide diuretics, thiazide-like diuretics and potassium-sparing diuretics, or combinations of any of the above.

In certain embodiments, the loop diuretics used in combination with compounds or compositions of the invention include, but are not limited to, bumetanide, ethacrynic acid, furosemide, torsemide, or combinations of any of the above.

In certain embodiments, the thiazide diuretics used in combination with compounds or compositions of the invention include, but are not limited to, epitizide, hydrochlorothiazide and chlorothiazide, bendroflumethiazide, methyclothiazide, polythiazide, or combinations of any of the above.

In certain embodiments, the thiazide-like diuretics used in combination with compounds or compositions of the invention include, but are not limited to, indapamide, chlorthalidone, metolazone, or combinations of any of the above.

In certain embodiments, the potassium-sparing diuretics used in combination with compounds or compositions of the invention include, but are not limited to, amiloride, triamterene, spironolactone, eplerenone, or combinations of any of the above.

In certain embodiments, a provided compound, or composition thereof, is administered in combination with a mechanical thrombectomy device, to a patient in need thereof. In certain embodiments, the mechanical thrombectomy device is a stroke thrombectomy device or a coil embolization device for cerebral aneurysm. In certain embodiments, such a device includes, but is not limited to, a coil retriever, an aspiration device or a stent retriever.

In certain embodiments, a combination of 2 or more therapeutic agents may be administered together with compounds or compositions of the invention. In certain embodiments, a combination of 3 or more therapeutic agents may be administered together with compounds or compositions of the invention.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the present invention, an additional therapeutic agent, and a pharmaceutically acceptable carrier, adjuvant, or vehicle.

The amount of both, a provided compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between about 0.001-100 mg/kg body weight/day of the additional therapeutic agent can be administered, or about 0.001 mg/kg to about 500 g/kg, or about 0.005 mg/kg to about 250 g/kg, or about 0.01 mg/kg to about 100 g/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of the present invention and one or more additional therapeutic agents. The therapeutic agent may be administered together with a compound of the present invention, or may be administered prior to or following administration of a compound of the present invention. Suitable therapeutic agents are described in further detail below. In certain embodiments, a compound of the present invention may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of the present invention may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In some embodiments, the present invention provides a medicament comprising at least one compound of the present invention or a pharmaceutically acceptable salt thereof and a pharmaceutically acceptable carrier.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1: Adenosine A1R/A3R Agonist I-1 (AST-004) Reduces Brain Infarction in a Nonhuman Primate Model of Stroke

### Nonstandard Abbreviations and Acronyms

A1R, adenosine A1 receptor
A2aR, adenosine A2a receptor
A2bR, adenosine A2b receptor
A3R, adenosine A3 receptor
ADC, apparent diffusion coefficient
AIS, acute ischemic stroke
AR, adenosine agonist
ASL, arterial spin labeling
CBF, cerebral blood flow
COMP, composite of all drug-treated groups
CSF, cerebrospinal fluid
Cmax, maximum concentration
DWI, diffusion-weighted imaging
Emax, maximum effect
FLAIR, fluid-attenuated inversion-recovery
HE, hemotoxylin-eosin staining
HERMES, Highly Effective Reperfusion Using Multiple Endovascular Devices
MABP, mean arterial blood pressure
MRI, magnetic resonance imaging
MCA, middle cerebral artery
MCAO, middle cerebral artery occlusion
MRA, magnetic resonance angiography
NDS, neurological deficit score
NHP, non-human primate
PEG, polyethylene glycol
PI, prediction interval
PK/PD, pharmacokinetics/pharmacodynamics
r-tPA, recombinant tissue plasminogen activator
RO, receptor occupancy
SEM, standard error of the mean
SMTP-7, Stachybotrys microspora triprenyl phenol-7
STAIR, Stroke Treatment Academic Industry Roundtable
tMCAO, temporary middle cerebral artery occlusion

### Introduction

Current acute ischemic stroke (AIS) therapy is limited to recanalization by thrombolysis or thrombectomy (reference 1). These therapies focus on restoring blood flow and oxygenation of hypoperfused tissue. Thrombolytics, however, can only be given to <5% of AIS patients within a limited time window post-occlusion, while thrombectomy requires access to the site of occlusion and is currently utilized in less than 20% of AIS patients (reference 2).

Pharmacotherapy that is both cerebroprotective and administrable to a majority of AIS patients in conjunction with recanalization is a major unmet need (reference 3). The vast majority of previous preclinical neuroprotection programs focused on efficacy evaluations in rodent models, with limited insights into drug distribution, target engagement and pharmacological response, perhaps inevitably leading to neutral or negative findings during clinical trials (reference 4). For these reasons, the international Stroke Treatment Academic Industry Roundtable (STAIR) has issued detailed guidelines on preclinical testing of potential AIS therapies, including evaluation of efficacy in both lyssencephalic and gyrencephalic species (reference 5). To date, we are only aware of the postsynaptic density protein-95 inhibitor nerinetide and the plasminogen modulator SMTP-7 that fulfilled STAIR guidelines, being evaluated in both rodent and nonhuman primate (NHP) stroke models, prior to initiation of clinical trials (references 6-9).

In the current study, we followed STAIR recommendations in evaluating the effect of I-1, a combined adenosine A1R/A3R receptor agonist, in a cynomolgus monkey model of transient middle cerebral artery occlusion (tMCAO) (references 10-12). In rodent stroke models, activation of central adenosine A1R or A3R leads to robust cerebroprotection, as defined by smaller brain lesion volumes compared to vehicle treatment (references 13-20). However, previous rodent studies have utilized treatment prior to initiation of the occlusion which leads to questions regarding translatablity of the findings to the clinical setting (reference 13).

The current study was designed to closely follow the average timelines for clinical study intervention in human AIS patients described in the HERMES (reference 21). Significant efficacy in terms of lesion growth rate inhibition and overall lesion size reduction were observed while no adverse effects on core physiological parameters became evident. Furthermore, clear relationships were observed between the observed efficacy and I-1 (AST-004) cerebrospinal fluid and plasma drug concentrations as well as estimated brain A1R/A3R receptor occupancy.

### Animals

A total of 25 adult male Macaca fascicularis macaques were used in this study, with ages ranging from 47-83 months and weights ranging from 2.7-5.4 kg.

The study was carried out in completely randomized (despite replacement subjects, see below) and blinded fashion. Subjects were randomized prior to treatment induction. Allocation concealment was maintained throughout study as separate blinded investigators were utilized for MCAO surgery, treatment induction and subsequent imaging and efficacy endpoint analyses. This was the first efficacy study of its type utilizing this primate model of transient MCAO with extensive imaging endpoints. Thus, we could not anticipate the size of potential treatment outcomes that would be required for a priori power and sample size calculations. The data generated in this exploratory study will allow us to conduct power and size calculations for future efficacy studies.

### Animal Quarantine and Stabilization

Prior to arrival at the animal facility, macaques were tested and were negative for Salmonella spp., Shigella spp., Yersinia spp., M. tuberculosis and Cercopithecine herpesvirus 1 (B virus). Individual health certifications were obtained for each macaque from the vendor (EBS, Inc., Hashimoto, Japan). Upon arrival, macaques used in the current study underwent a period of quarantine and observation for 14 days. The veterinarian and animal care staff performed daily cursory visual checks of each macaque assessing their physical state, general behavior, appetite, and excreta. Any physical, behavioral, appetite or excreta abnormalities were to be recorded in each macaque's individual health record and may have led to exclusion. No macaques used in the current study were observed for such abnormalities during the quarantine period or during the period of housing within the general colony. An exclusion criterion was abnormal cerebral anatomy (based on T2-weighted images), but no subjects needed to be excluded on this basis.

### Anesthesia

Macaques were initially sedated with ketamine HCl (10 mg/kg, i.m.) and treated with atropine sulfate (0.05 mg/kg, i.m.). Macaques were intubated, immobilized with 0.04-0.16 mg/kg (i.v.) vecuronium bromide and artificially ventilated. During the surgical procedure, animals were maintained on 0.8% isoflurane in a 7:3 mixture of N2O and O2. Before transient middle cerebral artery occlusion (tMCAO), the concentration of isoflurane was reduced to 0.5 0.6% and continued until 4 h after ischemia. During surgery, end-tidal CO2, body temperature, heart rate and blood pressure were monitored. During magnetic resonance imaging (MRI), macaques were anesthetized with 0.5-0.6% isoflurane. Sugammadex (8 mg/kg, i.v.) was administered to reverse the effect of vecuronium. Post-operative antibiotic (5 mg/kg enrofloxacin, i.m., twice daily, up to four days post-surgery), analgesic (0.01 mg/kg buprenorphine, i.m., twice daily, up to four days post-surgery) and prednisolone (1 mg/kg, i.m., once following surgery, then bidaily for the following four days) were administered, and animals were observed during recovery from anesthesia. Animals showing signs of severe pain or distress at any time, as determined by the attending veterinarians and their staff, were to be euthanized, but this was not required.

### Middle Cerebral Artery Occlusion and Reperfusion

Under deep anesthesia, the right eyeball was removed, and the orbital content was dissected and excised using a surgical microscope (KOM300, Konan Medical Inc., Hogo, Japan). A window of approximately 10 mm in diameter was opened just anterior to the foramen of the optic canal at the base of the skull, allowing identification of the right middle cerebral artery (MCA) main trunk beneath the dura. After opening the dura mater, tMCAO was performed using 2 microvascular clips (Sugita TII, 17-001-81, Mizuho Medical, Tokyo, Japan), one on the proximal part of the main MCA trunk and the other on the distal-to-orbitofrontal brunch. Four hours after tMCAO, these clips were removed. After visual confirmation of recanalization of MCA blood flow, the burr hole was closed using Clearfil New Bond (Kuraray Noritake Dental, Inc., Tokyo, Japan) and the orbital cavity was closed according to best veterinary practice.

### Physiological Parameter Assessment

Body weight, core body temperature, mean arterial blood pressure (MABP), heart rate, pO₂, pCO₂, sO₂ and blood pH were assessed prior to and after tMCAO at designated intervals in relation to reported normal physiological ranges for each parameter (references 22-23).

### Transient Middle Cerebral Artery Occlusion and Reperfusion

Transorbital transient MCA occlusion (tMCAO) was performed using 2 microvascular clips, one placed on the proximal part of the main MCA trunk and the other on the distal-to-orbitofrontal branch (references 10, 11, 24). Four hours after MCA occlusion, these clips were removed for recanalization. After visual confirmation of restituted MCA blood flow, the burr hole was closed using Clearfil New Bond (Kuraray Noritake Dental, Inc., Tokyo, Japan) and the orbital cavity was closed according to best veterinary practice.

### Magnetic Resonance Imaging

Serial coronal magnetic resonance imaging (MRI) of the brain (3 mm slice thickness) was performed 0.5, 1.5, 1.8, 3.5, 6.0, 24 and 120 h post-occlusion. Imaging sequences were (i) diffusion-weighted imaging (DWI), arterial spin labeling (ASL), (ii) magnetic resonance angiography (MRA), and (iii) fluid-attenuated inversion-recovery (FLAIR) T2-weighted imaging. Apparent diffusion coefficient (ADC) maps, cerebral blood flow (CBF), and perfusion deficit were generated with FuncTool Performance (GE Healthcare, Milwaukee, WI, USA) available on the MRI scanner console. Inhibition of lesion volume was considered the primary efficacy endpoint. Penumbral volume (mm³) was calculated by subtracting the lesion volume delineated from the DWI diffusion maps from the total calculated perfusion deficit.

### Measurement of Lesion Volume, Perfusion Deficit, and Penumbra Volume

### Measurement Details

The infarct (lesion) areas (mm²) of each coronal image were marked using OsiriX version 8.0.2 (Pixmeo SARL, Bernex, Switzerland). The infarct was manually delineated using DWI maps. The lesion volume (mm³) was calculated as the sum of the product of each section's infarct area and slice thickness (3 mm). In calculating infarct area and volume, no adjustments were made for potential edema. The perfusion deficit (mm³) was calculated from ASL maps as a reduction to <30% and <50% of the corresponding region of the contralateral side. For clarity, the perfusion deficit includes both the penumbra volume (hypo-oxygenated tissue) and lesion volume (necrotic tissue). Penumbral volume (mm³) was calculated by subtracting the lesion volume delineated from the DWI diffusion maps from the total calculated perfusion deficit.

### Control for Edema Effects on Volumetric Datasets

In general, we did not observe major edema build-up in post-reperfusion imaging in any subjects included in the study and final analysis. Lesion volume data from those subjects included in the final analysis were nevertheless controlled for a potential impact of the edema using Gerriets' method. Analysis confirmed that the impact of the edema on volumetric data was <1% and did not affect any of the performed analyses.

### Subject Exclusion by Lesion Volume

At 1.5 hours post-occlusion, the infarct volume for each macaque was calculated from the DWI and fell within the 90% prediction interval (PI) as determined by the linear regression model:

$$log ( lesion volume , {mm}^{3} ) = 5.141 + 0.6 \; 41 \times ( time after MCA occlusion , hours )$$

based on lesion volumes obtained over time from 3 macaques with a 3-hour occlusion and 6 macaques with a 4-hour occlusion (historical data, animals not included in this study). The 90% PI was calculated as:

$$PI = mean \pm {t}_{0.1 , df = n - 1} \times RSD \times sqrt ( 1 + 1 / n )$$

where the residual standard deviation (RSD) was 0.614, the number of observations (n) is 57 and the t-value (two-tailed) for the degree of freedom of n-1 and the significance level of 0.1 (t_{0.1, d.f.=n-1}) was 1.68. Thus, at 1.5 hours post-occlusion, the mean (90% P.I.) lesion volume was 447.0 mm³ (158.6-1259.4). Macaques with calculated infarct volumes falling either below or above the 90% PI were excluded at that point from treatment and excluded from the study.

### Drug Administration and Pharmacokinetic Sampling

Macaques received a bolus dose of vehicle (40% PEG400 in 0.9% saline) or I-1 into a saphenous vein 2 h after occlusion (2 h prior to restoration of MCA blood flow), followed immediately by a 22 h intravenous infusion. This dosing regimen was designed to rapidly achieve and maintain pre-determined plasma and cerebrospinal fluid (CSF) steady-state concentrations of I-1 based on its pharmacokinetics previously determined in näive and MCA occluded macaques (Tables A-C below).

TABLE A
Dose regimens, predicted plasma concentrations and resulting
measured average plasma and CSF concentrations of I-1.

| | | predicted | measured | Measured |
|---|---|---|---|---|
| Dose | bolus/infusion | I-1 *[Plasma] | I-1 *[Plasma] | I-1 *[CSF] |
| group | dose regimen | (ng/mL) | (ng/mL) | (ng/mL) |
| Very | 0.11 mg/kg; 0.06 | 35 | 32 ± 11 | 2.1 ± 0.4 |
| Low | mg/kg/h, 22 h | | | |
| Low | 0.47 mg/kg; 0.25 | 150 | 186 ± 24 | 8.8 ± 2.6 |
| | mg/kg/h, 22 h | | | |
| Mid | 1.7 mg/kg; 0.9 | 540 | 483 ± 23 | 16.8 ± 5.7 |
| | mg/kg/h, 22 h | | | |
| High | 5.2 mg/kg; 2.8 | 1660 | 1127 ± 246 | 108 ± 35 |
| | mg/kg/h, 22 h | | | |

*Plasma and CSF concentrations are means ± SEM of average concentrations over 22-hour infusion period, n = 4 per dose group.

TABLE B
Pharmacokinetic Parameters of Compound I-1 in Normal and tMCAO
Nonhuman Primates Following Intravenous Bolus Administration

| | Intravenous | | | | | Plasma; Brain |
|---|---|---|---|---|---|---|
| Dose | Bolus | C₀ | Vdss | Plasma Clearance | Half-Life | Unbound |
| Group | (mg/kg) | (ng/ml) | (L/kg) | (mL/min/kg) | (hr) | Fractions (%) |
| Normal | 0.5 | 329 ± | 3.1 ± | 29 ± | 1.3 ± | 26 ± 5; |
| (n = 3) | | 60 | 0.1 | 3 | 0.1 | 56 ± 5 |
| tMCAO | 0.5 | 544 | 3.0 | 28 | 1.5 | N.D. |
| (n = 1) | | | | | | |

Results are Means ± SEM
N.D. Not Determined

TABLE C
I-1 Plasma and CSF Pharmacokinetics and Predicted/Actual
Steady State Plasma Concentrations in tMCAO Cynomolgus
Monkeys Following Intravenous Bolus/Constant Rate Infusion
Regimens in a Pre-Efficacy Pharmacokinetic Study

| | | | Actual Time- | |
|---|---|---|---|---|
| | Bolus Dose | Predicted | Averaged | CSF/Plasma |
| | (mg/kg)/ | Steady-State | Steady-State | At 22-hour End |
| | Infusion | [Plasma] | [Plasma] | of Dosing |
| Group | (mg/kg/hr) | (ng/mL) | (ng/mL) | (%) |
| tMCAO | 0.11/0.0582 | 35 | 25 | 9.4 |
| (n = 1) | | | | |
| tMCAO | 1.7/0.9 | 540 | 363 | 8.4 |
| (n = 1) | | | | |
| tMCAO | 5.2/2.76 | 1660 | 1812 | 6.5 |
| (n = 1) | | | | |

During MRI, vehicle or I-1 were infused into the saphenous vein via a syringe pump (TOP5500E, TOP Corp., Tokyo, Japan). Infusion following MRI was maintained via a portable, programmable IPRECIO® Dual infusion pump (DMP-100; Primetech Corp., Tokyo, Japan) ported into a jugular venous line, both being secured in a jacket. Methods for pharmacokinetic sampling and bioanalytical analysis of plasma and CSF are described below.

### Drug Administration and Pharmacokinetic Sampling

Blood samples (1.0 mL) were withdrawn into heparinized tubes from either the opposite cephalic or the femoral vein at 3, 6, 24, 48, 72 hours and 5 days post-occlusion. Heparinized blood was centrifuged at 4° C. at 1,800×g for 10 min. In the same animals, 0.3-0.5 mL of cerebrospinal fluid (CSF) was withdrawn at 6 and 24 hours post-occlusion. Macaques were sedated with ketamine (10 mg/kg, i.m.), CSF was collected via puncture of the cisterna magna. Plasma and CSF samples were flash frozen in liquid nitrogen and then stored at −70 C until shipment for processing. Samples were shipped on dry ice. Plasma and CSF concentrations of I-1 were determined by LC/MS/MS utilizing standard curves (performed at the Department of Bio Research, Kamakura Techno-Science, Inc. Kanagawa, Japan). Lower limits of quantitation for compound I-1 were 1.0 ng/mL and 0.1 ng/mL for plasma and CSF, respectively.

### Neurologic Deficit Assessment

Twenty-four hours and 5 days after occlusion, neurologic deficits were scored using the Neurologic Deficits Score (NDS) as described elsewhere (reference 25). The NDS was considered as a secondary efficacy endpoint.

### Exclusion Criteria and Replacement Subjects

Exclusion criteria are based on comparison of infarct volumes to 90% prediction intervals (PI) generated from lesion volumes in preliminary studies. Based on these exclusion criteria, two macaques were found to have infarct volumes outside the 90% PI and were excluded and replaced. Subjects that died during the study were also replaced. Three subjects died following complications from MCAO surgery and were replaced. Other exclusion criteria comprised general health limitation prior to study induction and violation of species-specific ranges of physiological parameters on 3 consecutive time points. No animals needed to be excluded based on these criteria.

### Statistics

The mean and standard error of the mean (SEM) were calculated using Microsoft Excel 2016 (Microsoft Corporation). Statistical analyses were performed using SAS Analytical Pro version 9.4 (SAS Institute, Tokyo, Japan) and EXSUS version 8.1 (CAC Croit Corp., Tokyo, Japan). Additional statistical analyses were performed with Prism 4.02 (GraphPad Software, San Diego, CA). Prior to the t-test of the composite and control groups, the F-test was conducted to confirm equal variances between the control and composite groups. Then the t-test, equal variances, was conducted on the composite versus the control group and if positive for the endpoint, the individual dose groups were examined and discussed for relevance. These individual analyses will not alter a conclusion about the statistical significance of the composite and are considered descriptive analyses, not tests of hypotheses. Statistical significance was set at p<0.05, with trends towards statistical significance defined as 0.05≤p<0.1.

### Results

### Physiological Parameters and Baseline Measurements

Physiological parameters were measured prior to tMCAO (baseline) and throughout the study. There were no clinically relevant differences between vehicle-treated and I-1-treated groups for any parameter at baseline or during the study period. All parameters predominantly stayed within normal physiological ranges with occasional minor and transient deviations, not triggering pre-set exclusion criteria.

### I-1 Slows Ischemic Lesion Growth

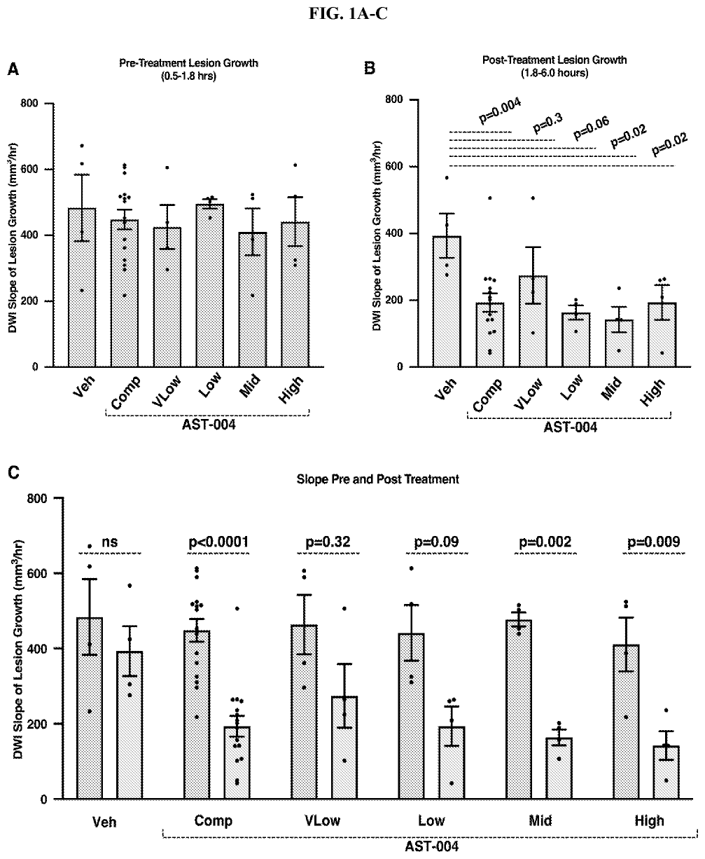

I-1 administration resulted in a rapid decrease in lesion growth rate (i.e. decreased slope) compared to both vehicle and pre-I-1 treatment growth rates. The slope of lesion growth was calculated as a measure of lesion growth rate, comparing the linear phases of the lesion growth curve during the pre-drug initiation (0.5-1.8 h) and post-drug initiation (1.8-6.0 h) periods (FIG. 1A, B). During the pre-drug initiation period, slopes of lesion growth were not different between vehicle- and I-1-treated groups. However, after initiation of I-1 treatment, the composite group slope was less than that of the vehicle group (p=0.004). The slopes of lesion growth for I-1 dose groups were smaller than that of the vehicle-treated group with statistical significance achieved in the Mid and High dose groups (p=0.02), and with a trend (p=0.06) observed in the Low dose group. In addition, when the post-drug slopes of lesion growth were compared to their own pre-drug slopes, the rate of increase of the composite group after I-1 treatment was significantly less than the rate of increase before I-1 treatment (p≤0.0001). Similar results were observed for the Mid and High dose groups (p≤0.002 and p≤0.009, respectively; FIG. 1C). These data suggested that I-1 activation of the adenosine A1 and A3 receptors led to a significant reduction in the rate of lesion growth following tMCAO.

### I-1 Preserves the Penumbra and Reduces Overall Stroke Volumes

Initial measures of cerebral perfusion deficits were determined at 0.5 h post-occlusion. As assessed by either <30% or <50% contralateral cerebral blood flow (references 11, 26-28), initial MCAO perfusion deficits were not significantly different between vehicle-treated or any I-1-treated dose group (p=0.63-0.97).

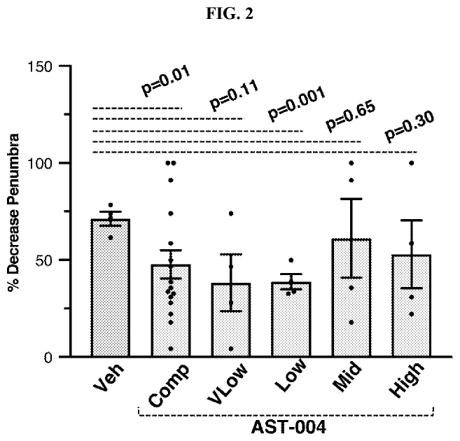

In general, the penumbra volume (as calculated by 30% perfusion deficit minus lesion volume) decreased during the ischemic period across all groups (FIG. 2). In the vehicle group, penumbra volume decreased by an average of 71% (at a rate of 604.5 mm³/h). In contrast, the I-1-treated composite decreased by 48% (at a rate of 296.0 mm³/h; p=0.01). Retention of penumbra volume was greatest in the Low dose group (p=0.001) compared to the vehicle group. The other I-1 treatment groups showed lower mean penumbra decrease rates compared to the vehicle group but high inter-subject variability may have prevented statistical significance in the Mid and High dose groups (p≥0.3).

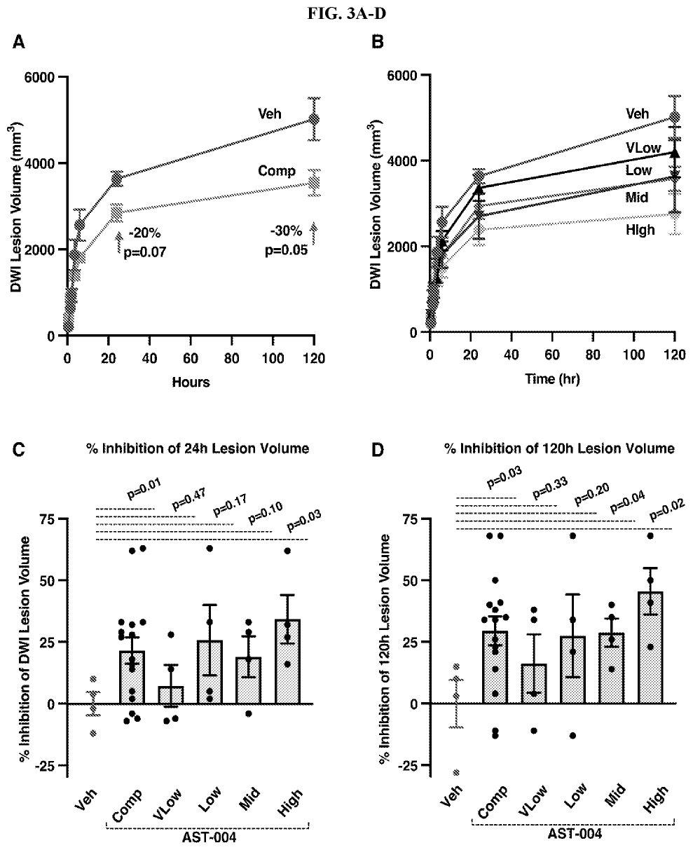

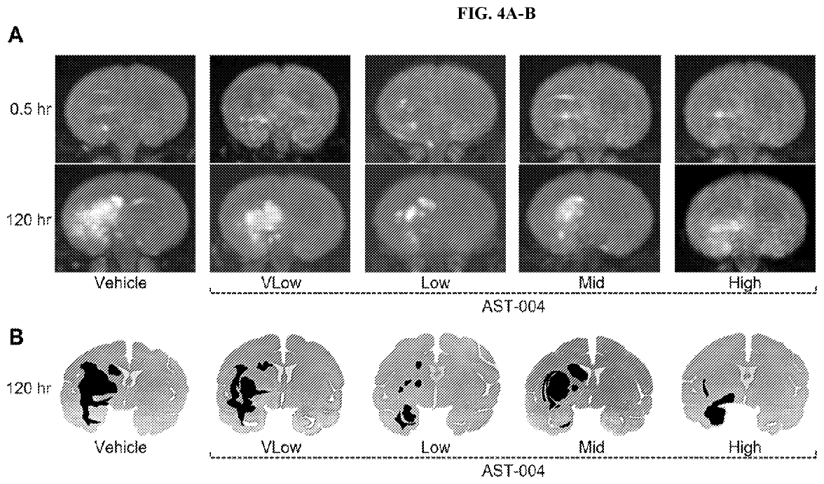

The reduction in the rate of lesion growth with I-1 treatment resulted in a significant inhibition of overall lesion volume as measured by DWI (FIG. 3A, B). At 24 h post-occlusion, overall lesion volume tended to be 20% smaller in the I-1 composite compared to vehicle treatment (p=0.07; FIG. 3C). However, overall lesion volume of the I-1 composite was 30% smaller than that of the vehicle group 120 h post-occlusion (p=0.05). Furthermore, the greatest inhibition of overall lesion volume was observed with Mid (p=0.04) and High (p=0.02) treatment (FIG. 3D). Overall lesion volume findings at 120 h were confirmed by histological lesion volume assessed in HE-stained brain sections (FIG. 4).

### I-1 Plasma and CSF Pharmacokinetics and Pharmacodynamics

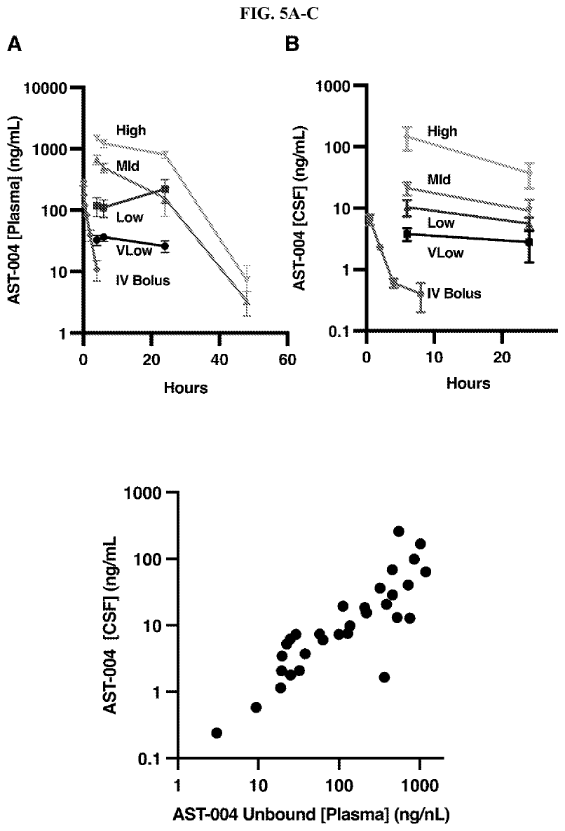

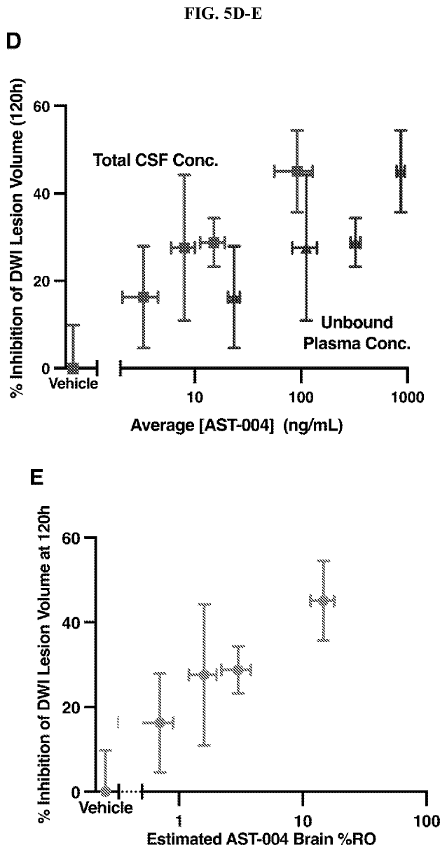

The I-1 dose levels in this study were designed to target specific multiples of plasma and CSF concentrations of I-1 and associated estimated brain adenosine A1 and A3 receptor occupancy, based on previous analyses of the pharmacokinetics of I-1 in naïve and tMCAO monkeys (Table B). Using the combined bolus/infusion regimen, average plasma and CSF concentrations of I-1 were within 2-fold of targets at all dose levels (Table, FIG. 5A, B and Table C) and remained within throughout the infusion period. Plasma concentration-time analyses confirmed the advantage of this dosing regimen to maintain targeted concentrations compared to a single, intravenous bolus in which I-1 plasma concentrations were below bioanalytical limits of quantitation 8 h post-dose. There was a good correlation between I-1 plasma and CSF concentrations, indicating a plasma/CSF ratio of approximately 10 (FIG. 5C).

Comparisons of average I-1 plasma and CSF concentrations to the primary efficacy endpoint of % inhibition of lesion volume demonstrated a linear relationship when analyzed by semi-logarithmic Eₘₐₓ plots (FIG. 5D). These analyses also demonstrate a linear relationship between lesion volume inhibition and brain A1R/A3R occupancy when plotted on an Eₘₐₓ curve (FIG. 5E).

### Secondary Endpoint

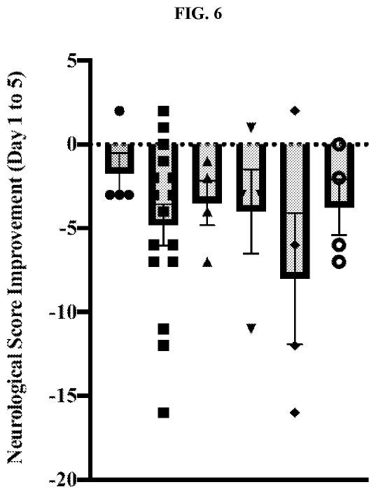

The current study did not have the appropriate power and duration to statistically assess neurological deficits following tMCAO. However, preliminary assessments were performed 1 and 5 days post-occlusion to identify any potential trends in neurological function. From study Days 1 to 5, improved but statistically non-significant (p=0.11) neurological function was observed in the I-1-treated composite compared to the vehicle-treated group (FIG. 6).

### Discussion

The current study demonstrated cerebroprotective efficacy of I-1, a dual agonist for the human A1R and A3R (reference 12), in a NHP model of 4 h transient cerebral ischemia. Compared to vehicle treatment, I-1 treatment reduced total infarct volume 24 h and 5 days after MCA occlusion. In addition to reduced total infarct volume, I-1 treatment reduced the rate of expansion of the infarct volume over time. Our findings suggest a cerebroprotective effect of I-1, supported by the finding that penumbra volume decline was reduced under I-1 treatment. In summary, the current findings suggest activation of A1R/A3R as a potential cerebroprotective strategy that could be utilized to prevent brain tissue necrosis and ultimately enhance functional outcome following an AIS.

Timely reperfusion of an occluded vessel will minimize brain tissue death and neurological impairment following AIS (reference 29). Although recanalization approaches such as thrombolysis by recombinant tissue plasminogen activator (r-tPA) or mechanical thrombectomy have revolutionized AIS treatment, they are restricted to relatively narrow time windows (less than 4.5 hours for r-tPA) and are restricted to selected patient populations exhibiting a large penumbra/core mismatch and accessible clots in operable large blood vessels (reference 29). A significant risk of cerebral hemorrhage is associated with delayed r-tPA treatment, and r-tPA is contraindicated for use in non-thrombotic strokes. This limits usage to a small percentage of stroke patients. A treatment that protects brain tissue from hypoxic insult and is not restricted to these narrow time windows would be of immense value in the treatment of stroke. Moreover, a treatment that has the potential to immediately slow penumbra decline before recanalization would widen the therapeutic time window for both thrombolysis and thrombectomy, increasing the number of eligible patients for these interventions and lowering the severity of AIS (reference 3).

### Basic Considerations and Pharmacokinetics

Extracellular brain adenosine concentrations significantly increase after the onset of ischemic stroke (references 30, 31). Activation of the four G-protein-coupled adenosine receptors, A₁R, A2aR, A2bR and A3R, plays important roles in both neuroprotection and neurodegeneration (reference 32). Activation of A2aR can lead to neurodegeneration through a glutamate receptor-mediated pathway. Activation of A2bR can lead to neurodegeneration through promotion of neuroinflammation, although research to date for this receptor is contradictory, with examples of both A2bR agonism and antagonism leading to cerebroprotection (references 33, 34). Neuroprotective effects are observed following activation of A₁R and A3R (references 13-20, 35). Thus far, there has been limited progress in developing therapeutics based on high-affinity adenosine receptor agonism for AIS, for several reasons. First, data suggests A₁R and A3R are susceptible to rapid desensitization by potent agonists including their endogenous ligand adenosine (references 36, 37). Second, high-affinity, full A₁R agonists have unacceptable peripheral cardiovascular side effects, including bradycardia and hypotension, due to vascular A₁R activation (references 38, 39). Moreover, clinical evaluations of high-affinity A3R agonists in AIS were likely limited by problematic chemical properties of previously synthesized nucleoside ligands, including poor brain distribution (references 40-43), low unbound brain concentrations preventing adequate target engagement (references 44, 45), as well as the aforementioned tendency to rapidly desensitize the receptor. An ideal AR agonist should exhibit excellent distribution in brain tissue and avoid potential adverse cardiovascular effects, for example with either lower-affinity or partial agonism, attributes that could also decrease the potential for receptor desensitization. Thus, the current study not only evaluated a potential cerebroprotective effect of I-1, but also carefully monitored subjects for any possible adverse cardiovascular side effects following systemic administration.

I-1 demonstrated good brain distribution, plus a high free fraction in both plasma and brain tissue. Cerebrospinal fluid drug concentration is an established proxy for unbound drug brain concentration that interacts with central receptor targets (references 46, 47). The unbound brain concentrations and resulting brain receptor occupancy at the A₁R and A3R can be estimated using receptor affinity data and simple mass action equations (references 48, 49). In previous studies with neonatal pigs, we demonstrated that I-1 CSF concentrations were equivalent to unbound I-1 brain extracellular fluid concentrations as determined via in situ equilibrium dialysis probes (reference 35). Accordingly, during the 22 h infusion of I-1, sufficient CSF concentrations were available to provide coverage of central A₁R and A3R in the macaque. Measurable concentrations of I-1 were found in plasma 24 h following termination of Mid and High dose infusions, suggesting prolonged presence of significant I-1 concentrations in the brain.

I-1 administration did not result in significant alterations in MABP or heart rate as would be expected with systemic administration of a full A₁R agonist (references 38, 39). In addition, no preset physiological criteria were violated for changes to body temperature, MABP pressure, heart rate, pO₂, pCO₂, sO₂ or pH. Occasional deviations of these parameters from the normal range could have been stroke- or procedure-related as such deviations were observed in both vehicle- and I-1-treated macaques. These data suggest a lack of significant adverse side effects following sustained activation of A₁R and A3R and further suggests that I-1 can be safely evaluated in human patients.

### I-1-Mediated Cerebroprotection

The penumbra eventually turns into infarcted tissue if timely revascularization does not occur. Thus, preservation or decelerating the loss of viable tissue in the penumbra during occlusion is a key objective of contemporary cerebroprotective agents (reference 3). Potential cerebroprotective agents have been previously tested in rodent models of tMCAO, but few have examined the effects of these agents on the penumbra, due in part to limited access to small animal MRI needed to visualize the diffusion-perfusion mismatch. Studies in large animals, including NHPs, have mostly relied on final lesion volume as a neuroanatomical indicator of neuroprotection (references 7, 24). The current study utilized the change in penumbra volume over time as an indication of cerebroprotection. The smaller infarct volumes observed at 24 h and 5 days following I-1 treatment appear as reductions in the rate of penumbra volume loss over time. Indeed, reduced penumbra volume loss in the composite and Low dose groups (p≤0.01) were observed. While there were lower mean differences in penumbra volume loss in all other I-1-treated groups, these differences were not statistically significant. Nonetheless, the data obtained are very encouraging, but could require larger cohorts for verification.

Decreased lesion growth rates are strong indicators of cerebroprotection. After occlusion but prior to treatment (0.5 h to 1.8 h post-occlusion, before infusion), the rates of lesion growth between vehicle- and I-1-treated groups were similar. However, compared to vehicle treatment, the rates of lesion growth after the onset of I-1 treatment were significantly lower. Decreased rates of infarct growth, in turn, resulted in significantly smaller infarct volumes at Day 5. Future studies using higher resolution in vivo imaging studies or absolute quantification of cerebral blood flow could identify specific neuroanatomical regions in the penumbra during tMCAO that benefit from therapeutic intervention (reference 50).

There is high sequence homology between NHP and human A₁R and A3R (reference 51), and the affinity of I-1 for the human A₁R and A3R is similar (reference 12), so it is expected that the calculated receptor occupancy values should be highly similar for both adenosine receptors in the NHP and human brain. On Eₘₐₓ plots of effect (% inhibition of lesion volume) versus matrix concentrations or estimated receptor occupancy, a linear relationship was observed across the I-1 dose regimens utilized in this study. Since Eₘₐₓ relationships are typically sigmoidal, these linear relationships present the intriguing possibility that the maximum efficacy of I-1 was not achieved in this study. Further studies will be needed to determine whether even higher levels of I-1 efficacy can be achieved in the NHP tMCAO model and to establish at what matrix concentrations and receptor occupancies Eₘₐₓ is reached.

### Limitations

Several limitations of the current study should be noted. First is the relatively small sample size. We chose the minimum sample in an attempt to reduce the number of animals used. Nevertheless, statistically significant positive outcomes were achieved in the primary outcome measure, suggesting a relatively large effect size. Second, there was high intersubject variability in many readout parameters. This may be due to the small sample size in treatment subgroups in combination with the relatively large scatter of individual data that may have ‘masked’ statistical significance. A larger scatter of data is typical for large animal models, which draw from a mixed population, and well reflects clinical reality (reference 52). Thus, we may assume that external validity of our study was good, although this means at the same time that certain endpoints may require larger group sizes for thorough assessment. Third, the overall observation period was relatively short. Additional changes may have occurred in some endpoints including the NDS in the long-term, but primary and other MRI-based key efficacy outcomes have benefitted from the chosen setup. Confirmation of our results in a larger cohort and primarily targeting functional outcome and long-term lesion organization may be warranted.

### CONCLUSION

To summarize, the current study investigated the effect of A1R/A3R activation via I-1, a novel adenosine A1R/A3R receptor agonist, in a NHP model of AIS with recanalization. Efficacy was observed in key outcome measures including rate of infarct volume growth, total lesion volume and retention of penumbral volume. Efficacy observed in these neuroanatomical outcome measures paralleled I-1 concentrations in plasma and CSF and, furthermore, align with estimated brain A1R/A3R receptor occupancy. Importantly, the receptor occupancy estimates associated with efficacy in this nonhuman primate model can be utilized to identify human clinical trial dose levels that yield similar levels of receptor occupancy, thus increasing the potential for translation in human stroke trials and ensuring that the pharmacological approach has been fully evaluated. These findings warrant further preclinical and clinical investigation of A₁R and A3R activation as a novel cerebroprotective strategy. The positive outcome regarding safety parameters also warrants early-stage clinical safety and efficacy testing in human patients.

### REFERENCES

1. Powers W J. Acute ischemic stroke. N Engl J Med. 2020; 383:252-260
2. Herpich F, Rincon F. Management of acute ischemic stroke. Crit Care Med. 2020; 48:1654-1663
3. Savitz S I, Baron J C, Fisher M, Stair X Consortium. Stroke Treatment Academic Industry Roundtable X: Brain cytoprotection therapies in the reperfusion era. Stroke. 2019; 50:1026-1031
4. Albers G, Ashwood, T, Barone, F C, Basta, S L, Bogousslavsky, J, Buchan, A M, Cady, W J, Chan, P H, Clemens, J A, Cox, B F, Craddock, R E, Cramer, S C. Recommendations for standards regarding preclinical neuroprotective and restorative drug development. Stroke. 1999; 30:2752-2758
5. Fisher M, Feuerstein G, Howells D W, Hum P D, Kent T A, Savitz S I, Lo E H. Update of the stroke therapy academic industry roundtable preclinical recommendations. Stroke. 2009; 40:2244-2250
6. Bratane B T, Cui H, Cook D J, Bouley J, Tymianski M, Fisher M. Neuroprotection by freezing ischemic penumbra evolution without cerebral blood flow augmentation with a postsynaptic density-95 protein inhibitor. Stroke. 2011; 42:3265-3270
7. Cook D J, Teves L, Tymianski M. Treatment of stroke with a PSD-95 inhibitor in the gyrencephalic primate brain. Nature. 2012; 483:213-217
8. Hill M D, Goyal M, Menon B K, Nogueira R G, McTaggart R A, Demchuk A M, Poppe A Y, Buck B H, Field T S, Dowlatshahi D, et al. Efficacy and safety of nerinetide for the treatment of acute ischaemic stroke (Escape-NA1): A multicentre, double-blind, randomised controlled trial. Lancet. 2020; 395:878-887
9. Suzuki E, Nishimura N, Yoshikawa T, Kunikiyo Y, Hasegawa K, Hasumi K.

Efficacy of SMTP-7, a small-molecule anti-inflammatory thrombolytic, in embolic stroke in monkeys. Pharmacol Res Perspect. 2018; 6:e00448
10. Hudgins W R, Garcia J H. Transorbital approach to the middle cerebral artery of the squirrel monkey: A technique for experimental cerebral infarction applicable to ultrastructural studies. Stroke. 1970; 1:107-111
11. Takamatsu H, Tsukada H, Kakiuchi T, Nishiyama S, Noda A, Umemura K.

Detection of reperfusion injury using pet in a monkey model of cerebral ischemia. J Nucl Med. 2000; 41:1409-1416
12. Liston T E, Hinz S, Muller C E, Holstein D M, Wendling J, Melton R J, Campbell M, Korinek W S, Suresh R R, Sethre-Hofstad D A, et al. Nucleotide P2Y1 receptor agonists are in vitro and in vivo prodrugs of A1/A3 adenosine receptor agonists: Implications for roles of P2Y1 and A1/A3 receptors in physiology and pathology. Purinergic Signal. 2020; 16:543-559
13. Chen G J, Harvey B K, Shen H, Chou J, Victor A, Wang Y. Activation of adenosine a3 receptors reduces ischemic brain injury in rodents. J Neurosci Res. 2006; 84:1848-1855
14. Fedorova I M, Jacobson M A, Basile A, Jacobson K A. Behavioral characterization of mice lacking the A3 adenosine receptor: Sensitivity to hypoxic neurodegeneration. Cell Mol Neurobiol. 2003; 23:431-447
15. Björklund O, Shang M, Tonazzini I, Dare E, Fredholm B B. Adenosine A1 and A3 receptors protect astrocytes from hypoxic damage. Eur J Pharmacol. 2008; 596:6-13
16. Martire A, Lambertucci C, Pepponi R, Ferrante A, Benati N, Buccioni M, Dal Ben D, Marucci G, Klotz K N, Volpini R, et al. Neuroprotective potential of adenosine A1 receptor partial agonists in experimental models of cerebral ischemia. J Neurochem. 2019; 149:211-230
17. Von Lubitz D K, Lin R C, Melman N, Ji X D, Carter M F, Jacobson K A. Chronic administration of selective adenosine A1 receptor agonist or antagonist in cerebral ischemia. Eur J Pharmacol. 1994; 256:161-167
18. Bischofberger N, Jacobson K A, von Lubitz D K. Adenosine A1 receptor agonists as clinically viable agents for treatment of ischemic brain disorders. Ann N Y Acad Sci. 1997; 825:23-29
19. Bozdemir E, Vigil, F. A., Bugay, V., Chun, S. H., Espinoza, L., Lorenzo, D., Holstein, D. M., Elliot, R., Sprague, C., Rule, G., Cavazos, J. E., Shapiro, M. S., Zamora, D., Brenner, R. and Lechleiter, J. D. Protective role of adora3 agonist AST-004 in blast and blunt TBI models. Military Health System Research Symposium. 2019
20. Sayre N L, Holstein, D., Stoveken, B. J., Sprague, S., Digicaylioglu, M., Liston, T., Korinek, W. S. and Lechleiter, J. D. Novel treatment reduces short and long term impacts of single and repetitive concussions in mice. Chronic Traumatic Encephalopathy (CTE) Conference. 2017
21. Goyal M, Menon B K, van Zwam W H, Dippel D W, Mitchell P J, Demchuk A M, Davalos A, Majoie C B, van der Lugt A, de Miquel M A, et al. Endovascular thrombectomy after large-vessel ischaemic stroke: A meta-analysis of individual patient data from five randomised trials. Lancet. 2016; 387:1723-1731
22. Yao J A, Feldman H S, Illenberger A, Littell T, Schnee L, Yates D. Evaluation of electrocardiograms recorded in cynomolgus monkeys with short- and long-term intracardiac lead implantations. J Pharmacol Toxicol Methods. 2009; 60:185-192
23. National Centre for the Replacement Refinement and Reduction of Animals in Research. The macaque website—physiological indicators. www.nc3rs.org.uk/macaques/welfare-assessment/physiological-indicators/. 2021.
24. Takamatsu H, Tsukada H, Noda A, Kakiuchi T, Nishiyama S, Nishimura S, Umemura K. FK506 attenuates early ischemic neuronal death in a monkey model of stroke. J Nucl Med. 2001; 42:1833-1840
25. Kito G, Nishimura A, Susumu T, Nagata R, Kuge Y, Yokota C, Minematsu K. Experimental thromboembolic stroke in cynomolgus monkey. J Neurosci Methods. 2001; 105:45-53
26. Sun Z, Zhang X, Zhang Y, Guo H, Zhang J, Yu C. Estimation of the ischemic penumbra based on C T perfusion a pilot study. Acad Radiol. 2010; 17:1535-1542
27. Hacke W, Albers G, Al-Rawi Y, Bogousslavsky J, Davalos A, Eliasziw M, Fischer M, Furlan A, Kaste M, Lees K R, et al. The desmoteplase in acute ischemic stroke trial (dias): A phase II MRI-based 9-hour window acute stroke thrombolysis trial with intravenous desmoteplase. Stroke. 2005; 36:66-73
28. Bivard A, McElduff P, Spratt N, Levi C, Parsons M. Defining the extent of irreversible brain ischemia using perfusion computed tomography. Cerebrovasc Dis. 2011; 31:238-245
29. Lansberg M G, O'Donnell M J, Khatri P, Lang E S, Nguyen-Huynh M N, Schwartz N E, Sonnenberg F A, Schulman S, Vandvik P O, Spencer F A, et al. Antithrombotic and thrombolytic therapy for ischemic stroke: Antithrombotic therapy and prevention of thrombosis, 9th ed: American college of chest physicians evidence-based clinical practice guidelines. Chest. 2012; 141:e601S-e636S
30. Phillis J W, Smith-Barbour, M., O'Regan, M. H., and Perkins, L. M. Amino acid and purine release in rat brain following temporary middle cerebral artery occlusion. Neurochemical Research. 1994; 19:1125-1130
31. Laghi Pasini F, Guideri F, Picano E, Parenti G, Petersen C, Varga A, Di Perri T. Increase in plasma adenosine during brain ischemia in man: A study during transient ischemic attacks, and stroke. Brain Res Bull. 2000; 51:327-330
32. Melani A, Pugliese A M, Pedata F. Adenosine receptors in cerebral ischemia. Int Rev Neurobiol. 2014; 119:309-348
33. Gu L, Huang B, Shen W, Gao L, Ding Z, Wu H, Guo J. Early activation of nSMase2/ceramide pathway in astrocytes is involved in ischemia-associated neuronal damage via inflammation in rat hippocampi. J Neuroinflammation. 2013; 10:109
34. Li Q, Han X, Lan X, Hong X, Li Q, Gao Y, Luo T, Yang Q, Koehler R C, Zhai Y, et al. Inhibition of tPA-induced hemorrhagic transformation involves adenosine a2b receptor activation after cerebral ischemia. Neurobiol Dis. 2017; 108:173-182
35. Kilbaugh T, Liston, T., Stolow, M., Hanlon, L., Bozdemir, E., Holstein, D., Kurbanov, A., Tunca, C., Poe, R., Lorenzo, D., Sethre-Hofstad, D., Lechleiter, J., and Margulies, S. Protective role of AST-004 in piglet model of blunt tbi—abstract. J. Neurotrauma. 2019; 36:10384
36. Klaasse E C, Ijzerman A P, de Grip W J, Beukers M W. Internalization and desensitization of adenosine receptors. Purinergic Signal. 2008; 4:21-37
37. Mundell S, Kelly E. Adenosine receptor desensitization and trafficking. Biochim Biophys Acta. 2011; 1808:1319-1328
38. Olsson R A, Pearson J D. Cardiovascular purinoceptors. Physiol Rev. 1990; 70:761-845
39. Roelen H, Veldman N, Spek A L, von Frijtag Drabbe Kunzel J, Mathot R A, A P I J. N6,C8-distributed adenosine derivatives as partial agonists for adenosine A1 receptors. J Med Chem. 1996; 39:1463-1471
40. Brodie M S, Lee K, Fredholm B B, Stahle L, Dunwiddie T V. Central versus peripheral mediation of responses to adenosine receptor agonists: Evidence against a central mode of action. Brain Res. 1987; 415:323-330
41. Gervitz L M, Lutherer L O, Hamilton M E, Fowler J C. Lack of central effects of peripherally administered adenosine A(1) agonists on synaptic transmission in the rat hippocampus. Brain Res. 2002; 951:141-145
42. Schaddelee M P, Read K D, Cleypool C G, Ijzerman A P, Danhof M, de Boer A G. Brain penetration of synthetic adenosine A1 receptor agonists in situ: Role of the rENT1 nucleoside transporter and binding to blood constituents. Eur J Pharm Sci. 2005; 24:59-66
43. Tosh D K, Rao H, Bitant A, Salmaso V, Mannes P, Lieberman D I, Vaughan K L, Mattison J A, Rothwell A C, Auchampach J A, et al. Design and in vivo characterization of A1 adenosine receptor agonists in the native ribose and conformationally constrained (n)-methanocarba series. J Med Chem. 2019; 62:1502-1522
44. Tosh D K, Padia J, Salvemini D, Jacobson K A. Efficient, large-scale synthesis and preclinical studies of MRS5698, a highly selective a3 adenosine receptor agonist that protects against chronic neuropathic pain. Purinergic Signal. 2015; 11:371-387
45. Wzorek J, Bednarek R, Watala C, Boncler M. Binding of adenosine derivatives to carrier proteins may reduce their antiplatelet activity. Biochem Pharmacol. 2020; 174:113827
46. Liu X, Smith B J, Chen C, Callegari E, Becker S L, Chen X, Cianfrogna J, Doran A C, Doran S D, Gibbs J P, et al. Evaluation of cerebrospinal fluid concentration and plasma free concentration as a surrogate measurement for brain free concentration. Drug Metab Dispos. 2006; 34:1443-1447
47. Liu X, Van Natta K, Yeo H, Vilenski O, Weller P E, Worboys P D, Monshouwer M. Unbound drug concentration in brain homogenate and cerebral spinal fluid at steady state as a surrogate for unbound concentration in brain interstitial fluid. Drug Metab Dispos. 2009; 37:787-793
48. Liu X, Vilenski O, Kwan J, Apparsundaram S, Weikert R. Unbound brain concentration determines receptor occupancy: A correlation of drug concentration and brain serotonin and dopamine reuptake transporter occupancy for eighteen compounds in rats. Drug Metab Dispos. 2009; 37:1548-1556
49. Watson J, Wright S, Lucas A, Clarke K L, Viggers J, Cheetham S, Jeffrey P, Porter R, Read K D. Receptor occupancy and brain free fraction. Drug Metab Dispos. 2009; 37:753-760
50. Werner P, Saur D, Zeisig V, Ettrich B, Patt M, Sattler B, Jochimsen T, Lobsien D, Meyer P M, Bergh F T, et al. Simultaneous PET/MRI in stroke: A case series. Journal of Cerebral BloodFlow & Metabolism. 2015; 35:1421-1425
51. van der Putten C, Zuiderwijk-Sick E A, van Straalen L, de Geus E D, Boven L A, Kondova I, A P I J, Bajramovic J J. Differential expression of adenosine A3 receptors controls adenosine A2a receptor-mediated inhibition of TLR responses in microglia. J Immunol. 2009; 182:7603-7612
52. Herrmann A M, Meckel S, Gounis M J, Kringe L, Motschall E, Mulling C, Boltze J. Large animals in neurointerventional research: A systematic review on models, techniques and their application in endovascular procedures for stroke, aneurysms and vascular malformations. J Cereb Blood Flow Metab. 2019; 39:375-394
53. Gerriets T, Stolz E, Walberer M, Muller C, Kluge A, Bachmann A, Fisher M, Kaps M, Bachmann G. Noninvasive quantification of brain edema and the space-occupying effect in rat stroke models using magnetic resonance imaging. Stroke. 2004; 35:566-571.

### Example 2: Determination of I-1 CSF Concentrations in Phase I SAD/MAD Studies

This discussion summarizes the rationale for obtaining CSF concentrations of I-1 in clinical Single Ascending Dose (SAD) and Multiple Ascending Dose (MAD) studies, and how the information will be used to estimate central target receptor occupancy and Phase II doses.

### The Challenges of GPCR Agonism Versus Antagonism

The majority of pharmaceutical drug discovery and development is focused on some form of receptor antagonism or enzyme inhibition, i.e., blocking the effects of an endogenous ligand. Because of the tendency of enzymes and receptors to have large reserves, the endogenous ligand only needs to interact with a relatively small percentage of the receptor population in order to have an EC₅₀ or Eₘₐₓ, effect. What this means is that drug receptor antagonists or enzyme inhibitors need to block a large percentage of the drug target active sites in order to block the effects of the endogenous ligand; in other words, they need to have a high receptor occupancy in order to have the desired therapeutic effect. This exact required percentage of antagonism/inhibition can vary greatly by target, tissue, and disease state. But, in general, this requirement for high receptor occupancy for antagonists/inhibitors forces the medicinal chemist to design for high affinity—not only to ensure broad coverage of active sites, but to also ensure that this can be achieved at a reasonable dose and at a reasonable cost of goods.

Conversely, a smaller receptor occupancy may be effective in producing the desired therapeutic result in the case of receptor agonists. In some cases, a small percentage of receptor occupancy can yield an EC₅₀ or Eₘₐₓ effect, again due to the large receptor reserve of many receptors. This EC₅₀-associate receptor occupancy can be as low as <1% for some agonists. The required receptor occupancy can vary greatly between species, tissues, and diseases due to differences in agonist affinity, potency, receptor-effector coupling, etc.

The occupancy of the receptor is the first step towards pharmacological effect leading to efficacy (or toxicity). So, it is very important to understand the receptor occupancy at/near the target site in order to set efficacious doses.

### The Challenge of Measuring Target Engagement in CNS Programs

It follows that if we can determine the receptor occupancy in the target organ/compartment associated with efficacy, we can utilize that number as a guide for dose selection. In many therapeutic areas, sampling of an appropriate target tissue can be relatively straightforward. For drug targets in well-perfused organs, plasma concentrations of drug can be readily used to estimate receptor occupancy in the target organ.

CNS drug targets are a special challenge in this area. This is because of the blood-brain barrier and blood-spinal cord barrier that can prevent distribution of drugs into the CNS compartment. The degree of unbound drug distribution from blood or plasma into brain is difficult to predict and can vary by species. The field of CNS drug discovery/development is replete with failed drug candidates that were advanced based on assumptions of brain free fraction, distribution of drug into the brain or the belief that plasma drug concentrations correlated with brain drug concentrations.

For these reasons, Liu and coworkers at Pfizer began to assess how to predict receptor occupancy in the brain. See Liu X, et al., “Evaluation of cerebrospinal fluid concentration and plasma free concentration as a surrogate measurement for brain free concentration,” Drug Metab Dispos. 2006; 34:1443-144; Liu X, et al., “Unbound drug concentration in brain homogenate and cerebral spinal fluid at steady state as a surrogate for unbound concentration in brain interstitial fluid. Drug Metab Dispos. 2009; 37:787-793; and Liu X, et al., “Unbound brain concentration determines receptor occupancy: A correlation of drug concentration and brain serotonin and dopamine reuptake transporter occupancy for eighteen compounds in rats,” Drug Metab Dispos. 2009; 37:1548-1556. First, they demonstrated that CSF drug concentrations were a much better predictor of brain unbound drug concentrations than were total or unbound plasma drug concentrations—particularly for more hydrophilic compounds such as I-1. Next, they demonstrated that brain unbound drug concentrations could be used to predict brain receptor occupancy. Since it is not possible to take brain biopsies from human volunteers, the next question was whether CSF drug concentrations could also be used to predict brain receptor occupancy. The Liu research as well as others indicates that CSF drug concentrations are a reasonable surrogate for the unbound brain drug concentrations required to estimate brain receptor occupancy, and superior to plasma concentrations that had been relied upon up to that point in time.

Determination of CSF concentrations of compounds such as I-1 will therefore enable clinical studies. All research up to this point in time indicates that plasma drug concentrations are poor predictors of brain receptor occupancy, while CSF drug concentrations are the next best thing to experimentally determined brain unbound extracellular fluid concentrations.

### Receptor Occupancy Calculations

Receptor occupancy is a mass action relationship:

$$\% Receptor Occupancy = ( \frac{[ D ]}{[ D ] + {K}_{i}} ) * 100$$

The percentage of a receptor occupied by an agonist or antagonist is a function of the drug concentration and the drug affinity for the receptor target. The challenge is that drug concentrations at the target site (in this case, brain, and CSF) can vary by species, as can affinity for the drug target. Affinity of I-1 for its receptor target can vary by as much as 5- fold between species. Accordingly, for each species, it is necessary to determine the I-1 affinity for its target as well as the concentration of I-1 at each administered dose in a relevant matrix. Plasma concentrations have been demonstrated not to be useful in predicting brain target receptor occupancy, so the only available matrix is CSF.

### I-1 Receptor Occupancy To Date

We have used this approach with great success in preclinical species to date: mouse, rat, neonatal pig and nonhuman primate. This work not only validates the approach of selecting doses based on estimated brain receptor occupancy, but also demonstrates why we need to make these determinations in each species, including human.

In our rodent mouse and rat efficacy studies, we were able to directly determine brain unbound drug concentrations at efficacious doses. In those efficacy models, the estimated receptor occupancies associated with efficacy were around 1% or less. This demonstrates that there is likely a very large receptor reserve of the drug target in rodent brains.

Using this receptor occupancy estimate, we advanced to studies in neonatal pigs, targeting doses that would yield unbound brain and CSF concentrations that would provide a similar receptor occupancy to that associated with efficacy in the rodent models. In the pig, we had the advantage of conducting pharmacokinetic studies involving CSF collection, brain equilibrium dialysis to directly determine brain unbound extracellular fluid concentrations, plasma concentrations and finally total brain concentrations. This was a key study because it demonstrated that I-1 concentrations in the CSF were equal to unbound brain extracellular fluid concentrations. This was a key study to validate the use of I-1 CSF concentrations to estimate brain receptor occupancy. In the pig efficacy studies, we achieved efficacy at estimated receptor occupancies of around 2%.

We then proceeded to our key preclinical stroke efficacy model in nonhuman primates. Pre-efficacy pharmacokinetic studies obtained plasma and CSF pharmacokinetic data. The I-1 CSF concentrations were combined with receptor affinity data to estimate receptor occupancy, and the combination of CSF concentrations and associated receptor occupancies were used to set dose regimens for the efficacy study.

In the monkey, we observed efficacy starting at estimated receptor occupancies in the range of 34%. We also observed a correlation between unbound plasma concentrations and CSF drug concentrations—but we would not have known what that correlation was unless we had collected the CSF and determined the I-1 CSF concentrations. If we were to conduct additional primate efficacy studies, we would not have to take CSF concentrations because we would know the relationship between unbound plasma and CSF drug concentrations. It is important to note that in these preclinical efficacy studies, unbound plasma concentrations would have greatly overpredicted brain receptor occupancy and would have resulted in us dosing below the threshold of I-1 efficacy.

### Intended Clinical Applications

Through our preclinical pharmacokinetic and efficacy studies, we have demonstrated the value of determining I-1 CSF or brain unbound drug concentrations and using these data to estimate brain receptor occupancy for dose setting and translation between species. Our intent is to extend this practice into human clinical trials to set doses for Phase II.

Our goals are the following:
Determine I-1 concentration in the CSF at each Phase I dose level
Determine the relationship between I-1 unbound plasma and CSF concentrations so that in future studies, we can predict CSF exposure from just plasma concentration data
Measure how CSF drug concentrations accumulate with repeated dosing, and most importantly, determine the doses of I-1 in humans that provide CSF drug concentrations that lead to the receptor occupancies associated with efficacy in preclinical models to date

After determining the latter, we will use it to set the mid-dose of our Phase II studies. Knowing that there can be species differences in receptor-effector coupling, we would set doses above and below this target dose to increase our chances of observing efficacy at a range of receptor occupancies around the targeted receptor occupancy. Due to the high failure rates and expense of running clinical trials, the approach we have outlined above provides an important solution to the need for greater predictability in evaluating new drug candidates, such as I-1 and related compounds described above.

### Example 3: Plasma and Brain Pharmacokinetics and Brain Free Fraction of I-25 (R-PIA) in Rats

The compound R-phenylisopropyladenosine (R-PIA) is an N⁶-substituted adenosine analog. Roucher and coworkers studied the effects of administration of R-PIA in a rat model of cerebral ischemia, while MacGregor and coworkers studied its effects on kainic acid-induced hippocampal lesions and neurological effects in rats. See Roucher, P., et al., J Cereb Blood Flow Metab. 1991 May; 11(3):453-8; MacGregor, D. G., et al., Br J Pharmacol. 1993 September; 110(1): 470-476; MacGregor, D. G., et al., Br J Pharmacol. 1993 June; 109(2): 316-321. R-PIA is a high affinity A₁R agonist with good A3R affinity as well. Its A₁R Kᵢ is 1.2 nM and its A3R Kᵢ is 158 nM. However, it also has A2a affinity of 220 nM. A2a agonism has shown neurodegenerative effects in prior studies.

Roucher studied the metabolic effects of R-PIA by in vivo ³¹P NMR spectroscopy before, during, and after 30 min of reversible forebrain ischemia in the rat. R-PIA had no effect on cerebral metabolism before ischemia. During a 30-min ischemia, R-PIA reduced the decrease in phosphocreatine (43+/−11% of the control level at the end of ischemia vs. 27+/−9% in the reference group) and ATP (58+/−12% vs. 40+/−23%) and the increase in inorganic phosphate (672+/−210% vs. 905+/−229%). The intracellular acidosis elicited by ischemia was also less in the treated group (pH of 6.40+/−0.10 vs. 6.30±/−0.10). Recirculation was associated with a faster recovery of PCr, ATP, Pi, and pHi to control levels in the treated group than in the reference group. It was concluded that adenosine protects against ischemic injury by mechanisms that include metabolic protection.

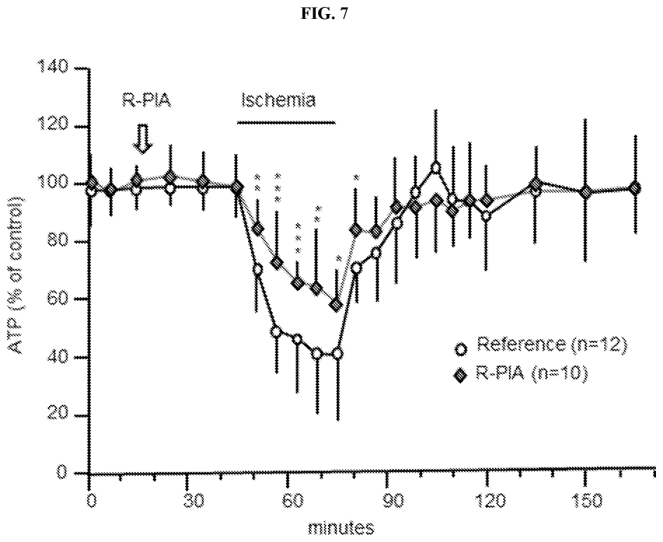

As part of the current study, we first reproduced the original Roucher results. As shown in FIG. 7, at a dose of 0.02 mg/kg, R-PIA demonstrated partial reversal of the decline in ATP during ischemia. Roucher does not describe the pharmacokinetics, brain concentrations, or target engagement of R-PIA.

We therefore performed the same study using ³¹P-ATP and measured the receptor engagement in the brain associated with the beneficial ATP effects. The estimated RO % were as follows: 16% RO for A₁R and 1% RO for A3R. The significance of this result is that, for the agonist R-PIA, A₁R and A3R receptor occupancies in the range of 1-16% resulted in significant reversal of ATP decline in this rodent model of cerebral ischemia. The estimated receptor occupancy for compound I-1 associated with significant efficacy in the NHP stroke study was also 1-15% (Low, Mid, High Doses).

These results continue to support the hypothesis that A1R/A3R agonism at relatively low receptor occupancy results in maintenance of brain ATP production and efficacy. Therefore, dosing with A1R/A3R agonists such as R-PIA and compound I-1 at doses that result in estimated brain receptor occupancies in the range of 15% is expected to show efficacy in both animals and humans.

### Example 4: Pharmacokinetics of Compounds Following Intraperitoneal Administration to Mice

Examples 1 and 2 of U.S. Pat. No. 9,789,131, which is incorporated herein by reference, describe assays for determining the plasma and brain concentrations of certain compounds following intraperitoneal administration of the compounds to mice at a dose used in mouse photothrombosis and traumatic brain injury models. Compounds described herein may be evaluated using such assays or similar variants thereof.

### Example 5: Plasma and Brain Binding of Test Compounds in Mice

Example 3 of U.S. Pat. No. 9,789,131, which is incorporated herein by reference, describes assays for determining the plasma and brain free fraction of certain compounds such as I-1. Compounds described herein may be evaluated using such assays or similar variants thereof.

### Example 6: In Vitro Stability and Metabolism of Test Compounds in Mouse and Human Blood and Plasma

Example 4 of U.S. Pat. No. 9,789,131, which is incorporated herein by reference, describes assays for determining the in vitro stability and metabolic fate in mouse and human blood and plasma of certain compounds such as I-1. Compounds described herein may be evaluated using such assays or similar variants thereof.

### Example 7: Neuroprotective Efficacy of Test Compounds after TBI in Mice

Example 5 of U.S. Pat. No. 9,789,131, which is incorporated herein by reference, describes assays for determining the efficacy of certain compounds such as I-1 in inducing neuroprotection in mice subjected to traumatic brain injury (TBI). Compounds described herein may be evaluated using such assays or similar variants thereof. The assay procedure is reproduced below.

### Purpose

This study is designed to determine the neuroprotective efficacy of test compounds in mice subjected to traumatic brain injury (TBI) and to compare free mice treated with test compounds and an adenosine A₃ receptor full agonist, Cl-IB-IMECA.

### Methods

Chemicals: Test compounds are prepared as described above. Cl-IB-IMECA is commercially available from Tocris Biosciences (Bristol, UK) and several other vendors. All other chemicals may be obtained from commercial vendors such as Sigma-Aldrich (St. Louis, MO).

Animals and traumatic brain injury (TBI): TBI is performed with a controlled closed skull injury model as described in Talley-Watts et al. 2012 (J. Neurotrauma 30, 55-66). Following the method described therein, a pneumatic impact device is used to generate a moderate TBI leaving the skull and dura matter intact. To achieve this, C57BL/6 mice are anesthetized with isoflurane (3% induction, 1% maintenance) in 100% oxygen. A body temperature of 37° C. is maintained using a temperature-controlled heated surgical table. A small midline incision is made on the scalp using aseptic surgical techniques. A 5 mm stainless steel disc is positioned on the skull and fixed using superglue on the right parietal bone between bregma and lamda over the somatosensory cortex. The mouse is then positioned on a stage directly under the pneumatic impact tip. A calibrated impact is delivered at 4.5 m/s at a depth of 2 mm which generates a moderate injury in the mouse. Scalp incisions are closed using 4-0 nylon braided suture and antibiotic ointment applied to the incision. Mice are placed in a Thermo-Intensive Care Unit (Braintree Scientific model FV-1; 37° C.; 27% O₂) and monitored until fully awake and moving freely. Thirty minutes following injury or sham (uninjured), mice are treated with either vehicle (saline), test compound, or control (Cl-IB-IMECA). Exemplary doses of test compound and Cl-IB-MECA are 0.16 and 0.24 mg/kg, respectively, each equivalent to equimolar doses of approximately 0.5 μmol/kg.

Western Blot Analysis for GFAP: At selected survival times, mice are anesthetized under isoflurane and sacrificed. The brain is removed and placed on ice for dissection into impacted and non-impacted brain hemispheres. The isolated tissue is rapidly homogenized in chilled homogenization buffer (0.32 M Sucrose, 1 mM EDTA, 1 M Tris-HCL pH=7.8) on ice using a Wheaton glass dounce (20 strokes). The homogenate is transferred to a 2 mL tube and centrifuged at 1000 g for 10 minutes at 4° C. and the supernatant is collected and analyzed. Protein concentration is determined by the BCA assay using a 1:50 dilution. 100 μg of protein is removed as an aliquot for each sample and Laemmli buffer containing P-mercaptoethanol added and the sample placed in a heat block for 3 minutes at 95° C. Samples are loaded on a 12% gel and run at 80 V for 20 minutes followed by 40 minutes at 130 V. Samples are transferred to nitrocellulose membrane at 100 V for 1 hour. The membrane is blocked with 5% milk in TBS-T for 30 minutes. GFAP (1:1000-Imgenex IMG-5083-A) is added and placed at 4° C. overnight. The membrane is washed with TBS-T three times for 10 minutes. Secondary antibody for GFAP (Donkey anti-rabbit HRP conjugated (ImmunoJackson Laboratories; 711-035-152; 1:20000) is applied at room temperature for 1 hour. The membranes are washed with TBS-T for 15 minutes (3 times) and developed using the Western Lightning Plus-ECL kit (PerkinElmer, Inc.) following manufacturer's directions.

### Results

Effective compounds (I-1 is known to be effective in this model) would be expected to reduce GFAP expression in the mouse brains following TBI. Glial Fibrillary acidic protein (GFAP) expression is used as a biomarker for reactive gliosis after TBI (Talley-Watts et al. 2012; Sofroniew, 2005). Western blot analysis will be performed for GFAP expression in Sham, TBI or TBI test compound-treated mice sacrificed at 7 days post-injury. First, western blot analysis confirms that TBI induces a significant increase in GFAP expression, both in the Ipsilateral (where the impact is centered) and contralateral sides of the brain at 7 days post-injury. GFAP expression is significantly lower in blots from mice treated with test compounds such as I-1, which are injected within 30 minutes of the initial trauma. For loading controls, beta-actin western blots are used. Typically, data will be averaged from 3 separate experiments and showing the relative change in GFAP/actin ratios (band intensities measured in Image J software).

Effective compounds (I-1 is known to be effective in this model) would be expected to reduce GFAP levels in mouse plasma following TBI. GFAP levels in the plasma have also been used as a biomarker for TBI, due to the breakdown of the blood brain barrier (BBB) after a trauma. Consequently, we will also collect plasma samples at day 7 from TBI mice. GFAP levels are easily detected at day 7 by western blot analysis.

Compound I-1 is a low-affinity (4900 nM) agonist of the A₃ receptor in the mouse. Conversely, Cl-IB-MECA is a high-affinity (0.18 nM) agonist in the mouse—the differences in affinity of these two compounds is approximately 25,000-fold. However, in the mouse photothrombotic stroke and TBI models, I-1 demonstrates significant efficacy that is blocked by the A₃ antagonist MRS1523, whereas Cl-IB-IMECA is either inactive (stroke) or weakly active. One potential explanation for this surprising result is based on ADME/PK data we have generated for I-1 and Cl-IB-IMECA. Cl-IB-IMECA is a lipophilic compound (cLogP approx 2.5) that is highly bound to plasma proteins (free fraction 0.002) and highly bound nonspecifically to brain tissue (free fraction 0.002). I-1 is a very hydrophilic compound (cLogP <0) that has a very large unbound fraction in plasma (0.74) and brain (0.13). Only unbound drug is available for distribution across membranes and interaction with receptors. Thus, despite its lower receptor affinity, the fraction of I-1 available to interact with the A₃ receptor in these mouse models is at least 1000-fold higher than that of Cl-IB-IMECA. These significant differences in compound physicochemical properties and ADME/PK characteristics may contribute to the non-obvious efficacy of I-1 and other compounds described herein as compared to Cl-IB-IMECA (and MRS5698, another lipophilic and highly-bound/high-affinity full A₃R agonist) in these mouse models. An alternative explanation is that compounds such as I-1 and other compounds shown in Table 1 act as dual A₃ and A₁ agonists or selective A₁ agonists.

### Example 8: Neuroprotective Efficacy of AST-004 After Stroke in Mice

Examples 6 and 7 of U.S. Pat. No. 9,789,131, which is incorporated herein by reference, describes assays for determining the efficacy of certain compounds such as I-1 in inducing neuroprotection in mice subjected to stroke. Compounds described herein may be evaluated using such assays or similar variants thereof. The assay procedure is reproduced below.

### Purpose

This study is designed to determine the neuroprotective efficacy of test compounds in mice subjected to stroke with and without the A₃ receptor antagonist MRS1523, and in comparison to full A3R agonists MRS5698 and Cl-IB-IMECA. MRS1523 has the following structure:

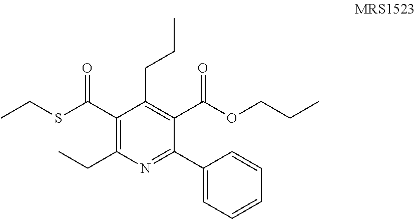 [SMILES: CCCOC(=O)c1c(-c2ccccc2)nc(CC)c(C(=O)SCC)c1CCC]

### Methods

Chemicals: Test compounds are prepared as described above. Cl-IB-MECA, MRS5698 and MRS2365 are commercially available from Tocris Bioscience (Bristol, UK) and several other vendors. All other chemicals may be obtained from Sigma-Aldrich (St. Louis, MO).

Photothrombosis-induced Stroke: Photothrombosis is performed as described in Zheng et al 2010 (PloS One 5 (12): e14401). In brief, Rose Bengal is a fluorescent dye that when injected into the vasculature and excited, generates singlet oxygen that damages the endothelial wall and induces a local thrombosis (clot). Using this technique, mice are given a 0.1 mL tail-vein injection of sterilized Rose Bengal (RB, Sigma, U.S.A.) in artificial cerebral spinal fluid (aCSF). The RB concentration is 20 mg/mL. A cortical region is centered in the imaging field and illuminated with a green laser (543 nm, 5 mW) using a 0.8-NA 40× water-immersion objective (Nikon, Tokyo). The clot formation is monitored in real time until the targeted vessel or downstream capillaries are firmly occluded. Stable clots are subsequently identified by a non-fluorescent vessel segmentation ending with highly fluorescent regions. In control experiments, either laser illumination or Rose Bengal itself did not lead to clot formation. Treatments, at doses such as 0.5 μmol/kg are introduced via intraperitoneal injections (i.p.). For experiments with A₃ receptor antagonist MRS1523, mice are administered intraperitoneal injections (2 mg/kg) at the 0 and 2 hour timepoints to ensure receptor antagonism throughout the course of the study.

Animals and Photothrombosis-induced Stroke: Stroke is performed as described in Zheng et al 2010 (PloS One 5 (12): e14401). Female C57Bl/6 mice (4-6 months) are used in this study. From the methods of this manuscript: Mice are anesthetized at 3% isoflurane with 100% oxygen and subsequently maintained at 1% isoflurane through a nosecone. Depth of anesthesia is monitored and regulated according to vital signs, pinch withdrawal and eye blinks. Body temperature is maintained at 37° C. by a feedback-controlled heating pad (Gaymar T/Pump). Vital signs including oxygen saturation, respiratory rate, and heart rate are continuously monitored by using the MouseOx system (STARR Life Sciences). The hair on each mouse's head is trimmed and a small incision is made in the scalp to expose the skull. A custom-made stainless steel plate is glued to the skull with VetBond Tissue Adhesive (3M, St Paul, MN). A cranial thinned-skull imaging window is created over the right primary somatosensory cortex (˜1.5 mm posterior to Bregma and 2 mm lateral from midline) depending on the experiment. In brief, a large region of the skull is first thinned with the electric drill and then further thinned with a surgical blade. The final thickness of the thinned skull is approximately 50 μm. After the cranial imaging window is created, mice are transferred to microscope stage and used for photothrombosis or imaging experiments. For the repeat imaging experiments, the plate is carefully detached from the skull and the scalp is sutured (Ethicon 6-0 sild suture). After each experiment, the mice are either returned to cages until the next timepoint or sacrificed. All procedures are approved by the Institutional Animal Care and Use Committee (IACUC) at University of Texas Health Science Center at San Antonio. Thirty minutes following stroke or sham (uninjured), mice are treated with either vehicle (saline) or test compound.

Post photothrombotic infarction evaluation. The size of cerebral infarcts is evaluated using 2,3,5-Triphenyltetrazolium chloride (TTC) staining as described in Zheng et al 2010 (PloS One 5 (12): e14401). In brief, RB-induced lesions in brain slices are stained with TTC. TTC is a colorless dye that stains healthy brain tissue red when reduced by the mitochondrial enzyme succinyl dehydrogenase (Bederson J B et al., 1986). The absence of staining in necrotic tissue is then used to define the area of a brain infarction. Mice are sacrificed by cervical dislocation, their brains removed and then placed in ice cold HBSS for 3 minutes. The brain is subsequently transferred to a brain mold (KOPF), sliced into 1 mm sections and immersed in 2% TTC (5 min) at 37° C. The sections are fixed in 10% buffered formaldehyde solution overnight at 4° C. Slices are imaged on a flatbed scanner (HP scanjet 8300) for analysis of the lesion size at 1200 dpi.

### Results

Multivessel photothrombotic strokes are induced in mice using tail-vein injected in conjunction with RB as described above. Within 30 minutes of clot formation, mice are injected intraperitoneally with either vehicle (saline control), or test compound. Twenty-four hours after the initial stroke, the brain infarction size is evaluated with TTC staining as described above.

It is expected that the A₃ receptor antagonist MRS1523 will inhibit neuroprotection of test compound after stroke. Multivessel photothrombotic strokes are induced in mice as described above. However, in this experiment, mice are treated with intraperatoneal injections of the A₃ receptor antagonist, MRS1523 (2 mg/kg) at the 0 and 2 hour timepoints to ensure receptor antagonism. Mice are then injected with either vehicle, test compound, MRS5698 or Cl-IBMECA within 30 minutes of clot formation at the concentrations described above. Twenty-four hours later, brain infarction sizes are evaluated with TTC staining.

### Example 9: Experimental Protocol for Determining Affinity, Agonism, and Biased Agonism of Compounds at Adenosine Receptors Such as the A3 Adenosine Receptor (A3R)

The following assays may be used to determine whether a disclosed compound exhibits agonism, partial agonism, or biased agonism (also known as functional selectivity or agonist trafficking) at the A₁, A₂A, or A₃ receptor. See Paoletta, S.; Tosh, D. K.; Finley, A.; Gizewski, E.; Moss, S. M.; Gao, Z. G.; Auchampach, J. A.; Salvemini, D.; Jacobson, K. A., “Rational design of sulfonated A3 adenosine receptor-selective nucleosides as pharmacological tools to study chronic neuropathic pain,” J. Med. Chem. 2013, 56, 5949-5963.

### Binding Studies of Human Adenosine Receptors (Includes A1, A2A and A3)

[³H]R-N⁶-Phenylisopropyladenosine ([³H]R-PIA, 63 Ci/mmol), [³H](2-[p-(2-carboxyethyl)phenyl-ethylamino]-5′—N-ethylcarboxamido-adenosine) ([³H]CGS21680, 40.5 Ci/mmol) and [¹²⁵I]N⁶-(4-amino-3-iodobenzyl)adenosine-5′—N-methyluronamide ([¹²⁵I]I-AB-MECA, 2200 Ci/mmol) were purchased from Perkin-Elmer Life and Analytical Science (Boston, MA). Test compounds were prepared as 5 mM stock solutions in DMSO and stored frozen. Pharmacological standards Cl-IB-MECA (A₃AR agonist), adenosine-5′—N-ethylcarboxamide (NECA, nonselective AR agonist) and 2-chloro-N⁶-cyclopentyladenosine (CCPA, A₁AR agonist) were purchased from Tocris R&D Systems (Minneapolis, MN).

Cell Culture and Membrane Preparation—CHO cells stably expressing the recombinant hA₁ and hA₃ARs and HEK293 cells stably expressing the hA_{2A}AR were cultured in Dulbecco's modified Eagle medium (DMEM) and F12 (1:1) supplemented with 10% fetal bovine serum, 100 units/mL penicillin, 100 μg/mL streptomycin, and 2 mol/mL glutamine. In addition, 800 μg/mL geneticin was added to the A_{2A} media, while 500 μg/mL hygromycin was added to the A₁ and A₃ media. After harvesting, cells were homogenized and suspended in PBS. Cells were then centrifuged at 240 g for 5 min, and the pellet was resuspended in 50 mM Tris-HCl buffer (pH 7.5) containing 10 mM MgCl₂. The suspension was homogenized and was then ultra-centrifuged at 14,330 g for 30 min at 4° C. The resultant pellets were resuspended in Tris buffer, incubated with adenosine deaminase (3 units/mL) for 30 min at 37° C. The suspension was homogenized with an electric homogenizer for 10 sec, pipetted into 1 mL vials and then stored at −80° C. until the binding experiments. The protein concentration was measured using the BCA Protein Assay Kit from Pierce Biotechnology, Inc. (Rockford, IL).

Binding assays: Into each tube in the binding assay was added 50 μL of increasing concentrations of the test ligand in Tris-HCl buffer (50 mM, pH 7.5) containing 10 mM MgCl₂, 50 μL of the appropriate agonist radioligand, and finally 100 μL of membrane suspension. For the A₁AR (22 g of protein/tube) the radioligand used was [³H]R-PIA (final concentration of 3.5 nM). For the A_{2A}AR (20 μg/tube) the radioligand used was [³H]CGS21680 (10 nM). For the A₃AR (21 g/tube) the radioligand used was [¹²⁵I]I-AB-MECA (0.34 nM). Nonspecific binding was determined using a final concentration of 10 μM NECA diluted with the buffer. The mixtures were incubated at 25° C. for 60 min in a shaking water bath. Binding reactions were terminated by filtration through Brandel GF/B filters under a reduced pressure using a M-24 cell harvester (Brandel, Gaithersburg, MD). Filters were washed three times with 3 mL of 50 mM ice-cold Tris-HCl buffer (pH 7.5). Filters for A₁ and A_{2A}AR binding were placed in scintillation vials containing 5 mL of Hydrofluor scintillation buffer and counted using a Perkin Elmer Liquid Scintillation Analyzer (Tri-Carb 2810TR). Filters for A₃AR binding were counted using a Packard Cobra II T-counter. The Kᵢ values were determined using GraphPad Prism for all assays.

### Binding Assay, Mouse A₃ Receptor

Similar competition binding assays may be conducted using HEK293 cell membranes expressing mARs using [¹²⁵I]I-AB-MECA to label A₁ or A₃ARs and [³H]CGS21680 to label A_{2A}ARs. IC₅₀ values were converted to Kᵢ values using known methods. Nonspecific binding was determined in the presence of 100 μM NECA.

### Functional Assay

cAMP accumulation assay: Intracellular cAMP levels in CHO cells expressing the recombinant hA₃AR were measured using an ELISA assay. Cells were first harvested by trypsinization. After centrifugation and resuspension in medium, cells were planted in 96-well plates in 0.1 mL medium. After 24 h, the medium was removed and cells were washed three times with 0.2 mL DMEM, containing 50 mM HEPES, pH 7.4. Cells were then treated with the agonist (10 μM NECA for hA₃AR) or test compound in the presence of rolipram (10 μM) and adenosine deaminase (3 units/mL). After 30 min forskolin (10 μM) was added to the medium, and incubation was continued for an additional 15 min. The reaction was terminated by removing the supernatant, and cells were lysed upon the addition of 100 μL of 0.1 M ice-cold HCl. The cell lysate was resuspended and stored at −20° C. For determination of cAMP production, 50 μL of the HCl solution was used in the Amersham cAMP Enzyme Immunoassay following the instructions provided with the kit. The results were interpreted using a SpectroMax M5 Microplate reader (Molecular Devices, Sunnyvale, CA) at 450 nm.

Similar cAMP assays were conducted with HEK293 cells expressing the mA₁AR or mA₃AR. HEK293 cells were detached from cell culture plates, resuspended in serum-free DMEM containing 25 mM HEPES (pH 7.4), 1 unit/ml adenosine deaminase, 4-(3-butoxy-4-methoxyphenyl)methyl-2-imidazolidone (Tocris, Ro 20,1724, 20 μM) and 300 nM 8-[4-[4-(4-chlorophenzyl)piperazide-1-sulfonyl)phenyl]]-1-propylxanthine (Tocris, PSB603, 300 nM) inhibit A_{2B}ARs expressed endogenously in HEK293 cells, and then transferred to polypropylene tubes (2×10⁵ cells/tube). The cells were co-incubated with forskolin (10 μM) and AR ligands for 15 min at 37° C. with shaking, after which the assays were terminated by adding 500 μL 1 N HCl. The lysates were centrifuged at 4000×g for 10 min. The cAMP concentration was determined in the supernatants using a competitive binding assay, as previously described (Nordstedt C, Fredholm B B, “A modification of a protein-binding method for rapid quantification of cAMP in cell-culture supernatants and body fluid,” Anal. Biochem. 1990; 189:231-234. [PubMed: 2177960]). EC₅₀ and Eₘₐₓ values were calculated by fitting the data to: E=Eₘᵢₙ+(Eₘₐₓ−Eₘᵢₙ)/(1+10^{x-logEC50}).

A3R binding of selected compounds. Using methods described herein, the affinity of selected compounds at the A₃ receptor was measured. Results are shown below.

TABLE D
Affinity of Selected Compounds at Human A₃R (n = 1)

| | MRS or Alternate | |
|---|---|---|
| Compound I-# | Compound # | Affinity (nM) |
| I-1 | AST-004 | 1490 |
| I-2 | MRS7711 | 909 |
| I-3 | MRS7662 | 504 |
| I-4 | MRS7706 | 1170 |
| I-5 | MRS7675 | 55 |
| I-6 | MRS7665 | 379 |
| I-7 | MRS7658 | 1053 |

### Determination of Biased Agonism at A3R.

Materials. Fluo-4, Dulbecco's modified Eagle's medium (DMEM), and penicillin-streptomycin may be purchased from Invitrogen (Carlsbad, CA). Adenosine deaminase (ADA) and hygromycin-B may be purchased from Roche (Basel, Switzerland). Fetal bovine serum (FBS) may be purchased from ThermoTrace (Melbourne, Australia). AlphaScreen SureFire extracellular signal-regulated kinases 1 and 2 (ERK1/2), Akt 1/2/3, and cAMP kits may be obtained from PerkinElmer (Boston, MA). Test compounds may be prepared as described herein. All other reagents may be purchased from commercial vendors such as Sigma-Aldrich (St. Louis, MO).

Cell Culture. The sequence of the human A₃R may be cloned into the Gateway entry vector, pDONR201, and then transferred in the Gateway destination vector, pEF5/FRT/V5-dest, using methods described previously (Stewart et al., 2009). A₃-FlpIn-CHO cells may be generated using methods described previously (May et al., 2007) and maintained at 37° C. in a humidified incubator containing 5% CO₂ in DMEM supplemented with 10% FBS and the selection antibiotic hygromycin-B (500 μg/ml). For cell survival, ERK1/2 phosphorylation, Akt 1/2/3 phosphorylation, and calcium mobilization assays, cells may be seeded into 96-well culture plates at a density of 4×104 cells/well. After 6 hours, cells are washed with serum-free DMEM and maintained in serum-free DMEM for 12-18 hours at 37° C. in 5% CO2 before assaying. For cAMP assays, cells may be seeded into 96-well culture plates at a density of 2×104 cells/well and incubated overnight at 37° C. in 5% CO₂ prior to assay.

Cell Survival Assays. Media is removed and replaced with HEPES-buffered saline solution (10 mM 4-(2-hydroxyethyl)-1-piperazineethanesulfonic acid (HEPES), 146 mM NaCl, 10 mM D-glucose, 5 mM KCl, 1 mM MgSO₄, 1.3 mM CaCl₂, and 1.5 mM NaHCO₃, pH 7.45) containing ADA (1 U/ml) and penicillin-streptomycin (0.05 U/ml) in the absence and presence of A₃R ligands. Plates are then maintained at 37° C. in a humidified incubator for 24 hours, after which 5 mg/ml propidium iodide is added to cells. Plates may be then read on an EnVision plate reader (PerkinElmer), with excitation and emission set to 320 nm and 615 nm, respectively. Data will be normalized to 100% cell survival and 0% cell survival, determined at t=0 hours in HEPES buffer and t=24 hours in Milli-Q water, respectively.

ERK1/2 and Akt 1/2/3 Phosphorylation Assays. A concentration-response curve of ERK1/2 and Akt 1/2/3 phosphorylation for each ligand may be performed in serum-free DMEM containing 1 U/ml ADA (5-minute exposure at 37° C.). Agonist stimulation may be terminated by removal of media and the addition of 100 ml of SureFire lysis buffer to each well. Plates are then agitated for 5 minutes. Detection of pERK1/2 may involve an 80:20:120:1:1 v/v/v/v/v dilution of lysate:activation buffer:reaction buffer:AlphaScreen acceptor beads:AlphaScreen donor beads in a total volume of 11 ml in a 384-well ProxiPlate. Plates may be incubated in the dark at 37° C. for 1 hour followed by measurement of fluorescence by an EnVision plate reader (PerkinElmer) with excitation and emission set to 630 nm and 520-620 nm, respectively. Detection of Akt 1/2/3 phosphorylation may employ a 40:9.8:39.2:1 v/v/v/v dilution of lysate:activation buffer:reaction buffer:AlphaScreen acceptor beads in a total volume of 9 1 in a 384-well Proxiplate. Plates may be incubated in the dark at room temperature for 2 hours, after which a 19:1 v/v dilution of dilution buffer:AlphaScreen donor beads may be added in a total volume of 11 μl. Plates may be incubated at room temperature for a further 2 hours, followed by measurement of fluorescence by an EnVision plate reader (PerkinElmer) with excitation and emission set to 630 nm and 520-620 nm, respectively. Agonist concentration-response curves are normalized to the phosphorylation mediated by 10% FBS (5-minute stimulation).

Calcium Mobilization Assays. Media may be removed from 96-well plates and replaced with HEPES-buffered saline solution containing 1 U/ml ADA, 2.5 mM probenecid, 0.5% bovine serum albumin (BSA), and 1 M Fluo4. Plates may be incubated in the dark for 1 hour at 37° C. in a humidified incubator. A FlexStation plate reader (Molecular Devices, Sunnyvale, CA) may perform the addition of HEPES-buffered saline solution in the absence and presence of agonist and measured fluorescence (excitation, 485 nm; emission, 520 nm) every 1.52 seconds for 75 seconds. The difference between the peak and baseline fluorescence may be measured as a marker for intracellular Ca²⁺ mobilization. A₃R agonist concentration-response curves may be normalized to the response mediated by 100 μM ATP to account for differences in cell number and loading efficiency.

Inhibition of cAMP Accumulation Assays. Media may be replaced with a stimulation buffer (140 mM NaCl, 5 mM KCl, 0.8 M MgSO₄, 0.2 mM Na₂HPO₄, 0.44 mM KH₂PO₄, 1.3 mM CaCl₂, 5.6 mM D-glucose, 5 mM HEPES, 0.1% BSA, 1 U/ml ADA, and 10 μM rolipram, pH 7.45) and incubated at 37° C. for 1 hour. Inhibition of cAMP accumulation may be assessed by preincubation of A₃-FlpIn-CHO cells with A₃R agonists for 10 minutes, after which 3 μM forskolin is added for a further 30 minutes. The reaction may be terminated by rapid removal of buffer and addition of 50 μl ice-cold 100% ethanol. Ethanol is allowed to evaporate before the addition of 50 μl detection buffer (0.1% BSA, 0.3% Tween-20, 5 mM HEPES, pH 7.45). Plates are agitated for 10 minutes, after which 10 μl lysate is transferred to a 384-well Optiplate. Detection may employ addition of a 5 μl 1:49 v/v dilution of AlphaScreen acceptor beads:stimulation buffer. Following this, a 15 μl 1:146:3 v/v/v dilution of AlphaScreen donor beads:detection buffer:3.3 U/μl biotinylated cAMP to form a total volume of 30 μl. The donor bead/biotinylated cAMP mixture may be equilibrated for 30 minutes prior to addition. Plates may be incubated overnight in the dark at room temperature, followed by measurement of fluorescence by an EnVision plate reader (PerkinElmer) with excitation and emission set to 630 nm and 520-620 nm, respectively. Agonist concentration-response curves may be normalized to the response mediated by 3 μM forskolin (0%) or buffer (100%) alone.

Molecular Modeling. Docking simulations can be performed for all the compounds investigated in this study using homology models of the human A₃R. In particular, three previously reported models can be used: a model entirely based on an agonist-bound hA₂AAR crystal structure (PDB ID: 3QAK), a model based on a hybrid A₂AAR-β2 adrenergic receptor template, and a model based on a hybrid A₂AAR-opsin template (β2 adrenoceptor X-ray structure PDB ID: 3 SN6; opsin crystal X-ray crystal structure PDB ID: 3DQB) (Tosh et al., 2012a). Models based on hybrid templates will show an outward movement of TM2 compared with the A_{2A}AR-based model. Structures of A₃R ligands may be built and prepared for docking using the Builder and the LigPrep tools implemented in the Schrödinger suite (Schrödinger Release 2013-3, Schrödinger, LLC, New York, NY, 2013). Molecular docking of the ligands at the A₃R models may be performed by means of the Glide package part of the Schrödinger suite. In particular, a Glide Grid may be centered on the centroid of some key residues of the binding pocket of adenosine receptors, namely, Phe (EL2), Asn (6.55), Trp (6.48), and His (7.43). The Glide Grid may be built using an inner box (ligand diameter midpoint box) of 14 Å×14 Å×14 Å and an outer box (box within which all the ligand atoms must be contained) that extends 25 Å in each direction from the inner one. Docking of ligands may be performed in the rigid binding site using the XP (extra precision) procedure. The top scoring docking conformations for each ligand may be subjected to visual inspection and analysis of protein-ligand interactions to select the proposed binding conformations in agreement with the experimental data.

Data Analysis. Statistical analyses and curve fitting may be performed using Prism 6 (GraphPad Software, San Diego, CA). To quantify signaling bias, agonist concentration-response curves may be analyzed by nonlinear regression using a derivation of the Black-Leff operational model of agonism, as described previously (Kenakin et al., 2012; Wootten et al., 2013; van der Westhuizen et al., 2014). The transduction coefficient, r/KA [expressed as a logarithm, Log (r/KA)], may be used to quantify biased agonism. To account for cell-dependent effects on agonist response, the transduction ratio may be normalized to the values obtained for the reference agonist, IB-MECA, to generate ALog(τ/KA). To determine the bias for each agonist at different signaling pathways, the ALog(τ/KA) will be normalized to a reference pathway, pERK1/2, to generate AALog(τ/KA). Bias may be defined as 10^{AALog(t/KA)} where a lack of bias will result in values that are not statistically different from 1, or 0 when expressed as a logarithm. All results may be expressed as the mean 6 S.E.M. Statistical analyses would involve an F test or a one-way analysis of variance with a Tukey or Dunnett's post hoc test, with statistical significance determined as P, 0.05.

### Example 10: Pharmacokinetics and Binding of AST-004 Following Intravenous Administration to Neonatal Pigs

### Purpose

This study is designed to determine the plasma, brain and CSF concentrations of test compounds following intravenous administration to neonatal pigs.

### Methods

Chemicals. Test compounds are prepared as described above.

Animals. Four-week old female neonatal pigs weighing approximately 7.5 Kg may be used for this study. Animals are equipped with brain microdialysis probes to obtain brain extracellular fluid samples for drug concentration determinations during the study.

Drug Administration: Test compound is solubilized in DMSO and then diluted in saline to prepare dosing solution. A 10 mL volume of dosing solution is administered by intravenous bolus administration to each neonatal pig (n=3).

Tissue Sampling: Blood samples are obtained at 0.25, 0.5, 1, 2, 4 and 6 hours post-dose. Brain extracellular fluid samples are obtained from implanted microdialysis probes at 1, 4 and 6 hours post-dose. Whole blood (1 mL) is obtained at each timepoint and placed in vacutainer tubes containing heparin and immediately centrifuged for preparation of plasma; plasma is stored at −80° C. Brain extracellular and cerebrospinal fluid samples are stored at −80° C. At the time of euthanasia (6 hours post-dose), cerebrospinal fluid samples are obtained and frozen, while brain samples from the cortex and hippocampus are obtained by decapitation, rinsed in ice-cold phosphate-buffered saline and weighed. Brain samples are then immediately flash-frozen in liquid nitrogen and stored at −80° C.

### Bioanalysis

Plasma, brain, brain extracellular fluid and cerebrospinal fluid concentrations of test compound are determined by LC-MS/MS utilizing tolbutamide as an internal standard. For each tissue matrix, standard curves are created and LLOQ/ULOQ concentrations determined.

For bioanalysis of brain concentrations of test compounds, brain samples are homogenized in ice-cold phosphate-buffered saline in a 4× dilution. Aliquots of the resulting diluted brain homogenate are treated with acetonitrile and analyzed by LC-MS/MS.

### Example 11: Plasma and Brain Binding of Test Compounds in Neonatal Pigs

### Purpose

This study is designed to determine the plasma and brain free fraction of test compounds in neonatal pigs.

### Methods

Chemicals. Test compounds may be prepared as described above. Analytical-grade sulfamethoxazole and warfarin may be obtained from commercial supplies such as Seventh Wave Laboratories (Maryland Heights, MO.). All other chemicals may be obtained from a commercial vendor such as Sigma-Aldrich (St. Louis, MO.).

Animals and Tissue Preparation. Plasma and brain samples from female neonatal pigs are obtained and stored at −80° C. until use.

Plasma ultrafiltrate blank samples are prepared by thawing frozen plasma and then pre-warming plasma in a humidified 5% CO₂ chamber at 37° C. for 60 minutes. Aliquots of 800 μl are transferred to Centrifree Centrifugal Filters (Ultracel regenerated cellulose (NMWL 30,000 amu) Lot R5JA31736) and centrifuged at 2900 RPM at 37° C. for 10 minutes; plasma water filtrates are collected and used in preparation of standards, blanks and QC standards.

Brains are weighed and homogenized with 1:9 phosphate-buffered saline, pH 7.4 using an Omni tissue homogenizer. Brains from four mice are homogenized, pooled and mixed to form one sample.

Plasma Binding Determination. Test compounds, sulfamethaxazole and warfarin are solubilized in DMSO and then diluted in 1:1 acetonitrile:water to prepare 100 μM dialysis stock solutions. Sulfamethaxazole and warfarin are utilized as study standards with known plasma binding values. Plasma samples are pre-warmed for 60 minutes in a humidified, 5% CO₂ incubator maintained at 37° C. Three ml aliquots of pre-warmed plasma are each spiked with test compound, sulfamethaxazole or warfarin using 100 μM stock solutions for each compound resulting in final test concentrations of 1 μM. Spiked plasma samples are incubated on a rotary mixer in a humidified 5% CO₂ chamber at 37° C. for a minimum of 60 minutes. After 60 minutes, three 800 μl aliquots of each sample are added to Centrifree centrifugal filters. The filters are subjected to centrifugation at 2900 rpm for 10 minutes at 37° C. Three 100 μl aliquots of residual plasma are collected along with ultrafiltrate for bioanalysis.

Brain Binding Determination: Test compounds, sulfamethoxazole and warfarin are solubilized in DMSO and diluted in 1:1 acetonitrile:water to prepare 100 μM dialysis stock solutions. Pooled homogenized brains are pre-warmed for 60 minutes in a humidified, 5% CO₂ incubator maintained at 37° C. Three ml aliquots of brain homogenate are each spiked with test compound, sulfamethaxazole or warfarin using the 100 μM stock solutions for each compound resulting in final spiked concentrations of 1 μM. Spiked pooled brain homogenates are placed on a Nutator mixer in a humidified, 5% CO₂ incubator at 37° C. for 60 minutes. After 60 minutes, three 800 μl aliquots of each sample are added to Centrifree centrifugal filters. The filters are subjected to centrifugation at 2900 rpm for 10 minutes at 37° C. Aliquots of residual brain homogenate and ultrafiltrate are collected for bioanalysis.

### Bioanalysis

Plasma and brain concentrations of test compounds in spiked plasma, brain homogenates and associated ultrafiltrates are determined by LC-MS/MS utilizing tolbutamide as an internal standard. Associated concentrations of sulfamethaxazole and warfarin are also determined by LC-MS/MS using standard conditions.

### Example 12: Pharmacological Characterization of Test Compounds

Test compounds are investigated in competition binding studies at human and mouse A₃ adenosine receptors recombinantly expressed in Chinese hamster ovary (CHO) cells using cell membrane preparations. [³H]NECA is employed as an A₃ agonist radioligand. The non-selective agonist NECA could be used because CHO cells do not natively express adenosine receptors. Concentration-dependent displacement of the radioligand by test compounds are determined.

Additionally cAMP experiments are conducted at CHO cells recombinantly expressing human A₃ or mouse A₃ adenosine receptors, respectively. The non-selective agonist NECA is used as a control.

See Alnouri M. W. et al., “Selectivity is species-dependent: Characterization of standard agonists and antagonists at human, rat, and mouse adenosine receptors,” Purinergic Signal. 2015, 11, 389-407. The same cell lines are used in the present and in the published study.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating an injury, disease, or disorder selected from a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of an agonist of the A₁ receptor (A₁R) and/or A₃ receptor (A₃R), or a pharmaceutically acceptable salt thereof or composition comprising the same, effective to reach 0.01-40% receptor occupancy (% RO) at brain or CNS A₁R and/or A₃R for a sufficient period of time to treat the condition.

2. A method of screening an A₁R, A₃R, or dual A₁R/A₃R agonist for efficacy in treating an injury, disease, or disorder selected from traumatic brain injury (TBI), stroke, a neurodegenerative condition, a heart or cardiovascular disease, and a condition associated with TBI, stroke, or the neurodegenerative condition, comprising administering to a subject in need thereof an amount of the A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R), wherein the A₁R, A₃R, or dual A₁R/A₃R agonist is effective at treating the injury, disease, or disorder if it reaches 0.01-40% RO at brain or CNS A₁R and/or A₃R.

3. (canceled)

4. The method of claim 1, wherein the amount of an agonist of the A₁ receptor (A₁R) and/or A₃ receptor (A₃R), or a pharmaceutically acceptable salt thereof or composition comprising the same, is effective to reach 1-15% receptor occupancy (% RO) at brain or CNS A₁R and/or A₃R.

5. A method of determining an effective dose for treating an injury, disease, or disorder selected from a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of an A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R).

6. A method of predicting effectiveness or predicting an effective dose for treating an injury, disease, or disorder selected from a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of a A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R).

7. A method of optimizing a treatment regimen for an injury, disease, or disorder selected from a traumatic brain injury (TBI), stroke, a neurodegenerative condition, a heart or cardiovascular disease, an addiction, an addictive disorder, and a condition associated with TBI, stroke, or the neurodegenerative condition, comprising administering to a subject in need thereof an amount of a A₁R, A₃R, or dual A₁R/A₃R agonist, or a pharmaceutically acceptable salt thereof or composition comprising the same; and determining the receptor occupancy (% RO) at brain or CNS A₁ receptors (A₁R) and/or A₃ receptors (A₃R).

8. The method of claim 1, wherein the injury, disease, or disorder is stroke.

9. A method of treating stroke, comprising administering to a subject in need thereof an amount of a compound selected from: 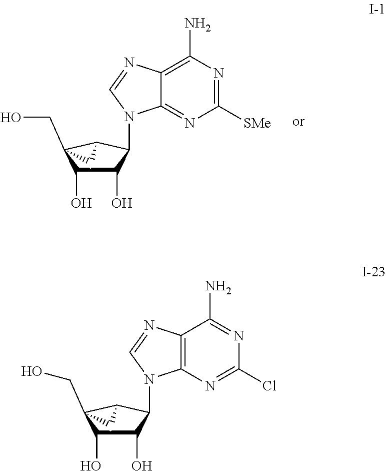
or a pharmaceutically acceptable salt thereof or composition comprising the same, effective to reach a plasma concentration of 30±20 ng/mL, 190±30 ng/mL, 480±100 ng/mL, 1100±300 ng/mL, 2500±400 ng/mL, or 3200±400 ng/mL for a sufficient period of time to treat the stroke.

10. The method of claim 9, wherein the stroke is selected from ischemic stroke, hemorrhagic stroke, subarachnoid hemorrhage, cerebral vasospasm, and transient ischemic attacks (TIA).

11. The method of claim 9, wherein the amount of the compound, or a pharmaceutically acceptable salt thereof or composition comprising the same, is effective to reach 1-15% receptor occupancy (% RO) at brain or CNS A₁ receptors (A₁R) and/or A₃ receptors (A₃R).

12. The method of claim 9, wherein the compound or pharmaceutically acceptable salt thereof or composition comprising the same is administered within 24 hours of the stroke.

13. The method of claim 9, wherein the compound or pharmaceutically acceptable salt thereof or composition comprising the same is administered orally.

14. The method of claim 9, wherein the compound or pharmaceutically acceptable salt thereof or composition comprising the same is administered by IV infusion.

15. The method of claim 9, wherein the compound or pharmaceutically acceptable salt thereof or composition comprising the same is administered initially as an IV bolus, followed by continuous IV infusion.

16. The method of claim 9, wherein the compound or pharmaceutically acceptable salt thereof or composition comprising the same is administered in an amount effective to reach a brain/plasma ratio of total drug of at least 0.01, at least 0.06, at least 0.1, or at least 0.2.

17. The method of claim 9, wherein the compound or pharmaceutically acceptable salt thereof or composition comprising the same is administered in an amount effective to reach a CSF concentration of 10-130 ng/mL.

18. The method of claim 9, wherein the compound or pharmaceutically acceptable salt thereof or composition comprising the same is administered in an amount effective to reach a plasma concentration of 1100±300 ng/mL.

19. The method of claim 1, wherein the agonist of the A₁ receptor (A₁R) and/or A₃ receptor (A₃R) or a pharmaceutically acceptable salt thereof or composition comprising the same, is administered orally.

20. The method of claim 1, wherein the agonist of the A₁ receptor (A₁R) and/or A₃ receptor (A₃R) or a pharmaceutically acceptable salt thereof or composition comprising the same, is administered initially as an IV bolus, followed by continuous IV infusion.

21. The method of claim 1, wherein the agonist of the A₁ receptor (A₁R) and/or A₃ receptor (A₃R) or a pharmaceutically acceptable salt thereof or composition comprising the same, is administered in an amount that results in a brain/plasma ratio of total drug of at least 0.01, at least 0.06, at least 0.1, or at least 0.2.

22. The method of claim 1, wherein the agonist of the A₁ receptor (A₁R) and/or A₃ receptor (A₃R) or a pharmaceutically acceptable salt thereof or composition comprising the same, is administered in an amount effective to reach a CSF concentration of 10-130 ng/mL.

23. The method of claim 9, wherein the compound or pharmaceutically acceptable salt thereof or composition comprising the same is administered in an amount effective to reach a plasma concentration of 1100±300 ng/mL.

24. The method of claim 1, wherein the agonist of the A₁ receptor (A₁R) and/or A₃ receptor (A₃R) is a compound selected from: 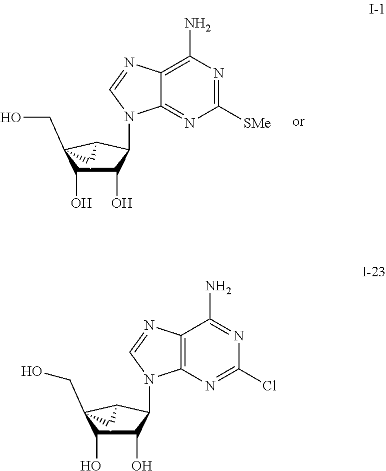
or a pharmaceutically acceptable salt thereof or composition comprising the same.

25. The method of claim 9, wherein the compound is: 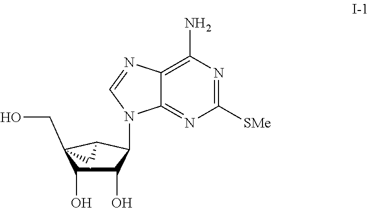
or a pharmaceutically acceptable salt thereof.

26. The method of claim 9, wherein the compound is: 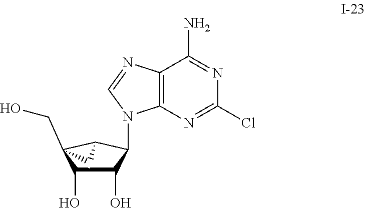
or a pharmaceutically acceptable salt thereof.

27. The method of claim 25, wherein the plasma/CSF ratio of compound I-1 concentrations is approximately 10.
